(19) United States
(12) Patent Application Publication
(10) Pub. No.: US 2023/0323480 A1
(43) Pub. Date: Oct. 12, 2023

(54) METHODS OF SCREENING FOR PLANT GAIN OF FUNCTION MUTATIONS AND COMPOSITIONS THEREFOR

(71) Applicant: The Regents of the University of California, Oakland, CA (US)

(72) Inventors: Dhruv Patel, Oakland, CA (US); Krishna K. Niyogi, Oakland, CA (US)

(73) Assignee: The Regents of the University of California, Oakland, CA (US)

(21) Appl. No.: 18/298,998

(22) Filed: Apr. 11, 2023

Related U.S. Application Data
(60) Provisional application No. 63/329,831, filed on Apr. 11, 2022.

(51) Int. Cl. C12Q 1/6895; C12N 15/11; C12N 9/22; C12N 15/82

(52) U.S. Cl. CPC C12Q 1/6895; C12N 15/11; C12N 9/22; C12N 15/8269; C12Q 2600/156; C12N 2800/80; C12Q 2600/13; C12Q 2600/158; C12N 2310/20

(57) ABSTRACT

The present disclosure relates to methods of screening for gain of function mutations in non-coding regions of target genes. The target genes may be NPQ genes, including photosystem II subunit S (PsbS), zeaxanthin epoxidase (ZEP), and violaxanthin de-epoxidase (VDE). The present disclosure further relates to methods of improving commercial crop plants or crop seeds by introducing gain of function mutations in non-coding regions of target genes, and to improved commercial crop plants or crop seeds produced by the methods.

### CROSS-REFERENCE TO RELATED APPLICATIONS

This application claims the benefit of U.S. Provisional Application No. 63/329,831, filed Apr. 11, 2022, which is hereby incorporated by reference in its entirety.

### REFERENCE TO AN ELECTRONIC SEQUENCE LISTING

The contents of the electronic sequence listing (794542001900SEQLIST.xml; Size: 171,268 bytes; and Date of Creation: Apr. 11, 2023) is herein incorporated by reference in its entirety.

### TECHNICAL FIELD

The present disclosure relates to methods of screening for gain of function mutations in non-coding regions of target genes. The target genes may be NPQ genes, including photosystem II subunit S (PsbS), zeaxanthin epoxidase (ZEP), and violaxanthin de-epoxidase (VDE). The present disclosure further relates to methods of improving commercial crop plants or crop seeds by introducing gain of function mutations in non-coding regions of target genes, and to improved commercial crop plants or crop seeds produced by the methods.

### BACKGROUND

Optimizing photosynthetic efficiency remains one of the largest remaining gaps in engineering sustainable and productive crop varieties (Long SP, Marshall-Colon A, Zhu X G. Meeting the global food demand of the future by engineering crop photosynthesis and yield potential. Cell. 2015; 161(1):56-66. doi:10.1016/j.cell2015.03.019). Previous research has shown that overexpression of genes involved in non-photochemical quenching (NPQ), the photoprotective process necessary to dissipate excess absorbed light energy, can increase the efficiency of photosynthesis, biomass accumulation (Kromdijk J, Glowacka K, Leonelli L, et al. Improving photosynthesis and crop productivity by accelerating recovery from photoprotection. Science. 2016; 354(6314):857-862), and water use efficiency (Glowacka K, Kromdijk J, Kucera K, et al. Photosystem II Subunit S overexpression increases the efficiency of water use in a field-grown crop. Nat Commun. 2018; 9(868):1-9. doi:10.1038/s41467-018-03231-x) of the model crop Nicotiana tabacum in field-relevant conditions. In addition, overexpression of genes involved in NPQ has been shown to increase seed yield in soybean (Souza A. P. D., Burgess S. J., Doran L., Hansen J., Manukyan L., Maryn N., et al. (2022). Soybean photosynthesis and crop yield are improved by accelerating recovery from photoprotection. Science 377, 851-854. doi: 10.1126/science.adc9831). However, these approaches relied on expression of foreign DNA, or transgenes, which incur additional regulatory complications and can be susceptible to gene silencing across generations (James V A, Avart C, Worland B, Snape J W, Vain P. The relationship between homozygous and hemizygous transgene expression levels over generations in populations of transgenic rice plants. Theor Appl Genet. 2002; 104(4):553-561. doi:10.1007/s001220100745),

CRISPR/Cas9 has dramatically expanded the capacity to produce targeted loss-of-function mutations. Recently, CRISPR/Cas9 editing of cis-regulatory elements has also demonstrated the utility of this approach to decrease rather than abolish gene expression through generation of partial loss-of-function alleles in tomato (Rodriguez-Leal D, Lemmon Z H, Man J, Bartlett M E, Lippman Z B. Engineering Quantitative Trait Variation for Crop Improvement by Genome Editing. Cell. 2017; 171(2):470-480.e8. doi:10.1016/j.cell2017.08.030) and maize (Liu L, Gallagher J, Arevalo E D, et al. Enhancing grain-yield-related traits by CRISPR-Cas9 promoter editing of maize CLE genes. Nat Plants. 2021; 7(3):287-294. doi:10.1038/s41477-021-00858-5). However, the ability to increase gene expression without the use of persistent transgenes is still lacking, and finer genome engineering by prime editing is bottlenecked by low editing, transformation, and regeneration efficiencies (Wada N, Ueta R, Osakabe Y, Osakabe K. Precision genome editing in plants: state-of-the-art in CRISPR/Cas9-based genome engineering. 2020:1-12). A robust pipeline to achieve such gains would rapidly accelerate what is possible by gene editing in crop plants, such as rice.

A method of screening plants for alleles with “weak effects” that can be screened for phenotypes such as increased yield, quality, or both is described in US patent application, US 2020/0199604. The methods therein rely upon use of plants having a first allele that is a hypomorphic allele or a null allele to allow for screening of the “weak effects”, which limits the utility of the screening method.

NPQ genes represent a promising avenue for genome editing approaches across plant species, as it is known that NPQ genes are found in all plants, and NPQ proteins are highly conserved in their function. There exists a need for genetic engineering approaches that alter endogenous gene expression in order to achieve agronomic gains without the need for stable transgenes. In particular, there exists a need for such approaches able to augment photoprotection (e.g., by targeting specific NPQ components) in order to improve photosynthetic processes and ultimately plant yield.

### BRIEF SUMMARY

In order to meet these needs, the present disclosure provides methods of screening for a gain of function mutation in a target gene such as a NPQ gene (e.g., PsbS, VDE, ZEP) to produce plants with improved photosynthetic processes. For example, the present disclosure uses this approach to target the rice Photosystem II Subunit S (OsPsbS1) gene, a core factor in high-light and fluctuating light tolerance, to generate mutants with increased OsPsbS1 expression, improved NPQ capacity, and putative increased water use efficiency.

An aspect of the disclosure includes methods of screening for a gain of function mutation in a target gene in a plant including: (a) generating a set of mutations in a non-coding sequence (NCS) of the target gene in a population of plant cells of the plant with one or more RNA-guided nucleic acid modifying enzymes targeting the target gene including one or more different guide RNAs; (b) regenerating the population of plant cells into two or more plants that are hemizygous for the mutation generated; (c) (1) selfing the two or more plants to generate offspring plants, and (2) optionally screening offspring plants that are homozygous for the mutation for screening in section (d); and (d) screening the offspring plants from step (c) to identify a gain of function mutation. An additional embodiment of this aspect further includes: (e) selecting a plant with the gain of function mutation, and (f) sequencing the target gene to identify the gain of function mutation. In a further embodiment of this aspect, which may be combined with any of the preceding embodiments, the one or more RNA-guided nucleic acid modifying enzymes is expressed from an expression vector including a selectable marker and the screening in step (d) includes screening for plants lacking the selectable marker. In another embodiment of this aspect, which may be combined with any of the preceding embodiments, the gain of function mutation induces overexpression of the target gene. In a further embodiment of this aspect, overexpression of the target gene is in the morning. In still another embodiment of this aspect, overexpression of the target gene is not constitutive, and/or the plant has a constitutive phenotype. In yet another embodiment of this aspect, which may be combined with any of the preceding embodiments, the target gene induces a phenotype associated with one or more of photosynthetic efficiency, photoprotection efficiency, non-photochemical quenching, photosynthetic quantum yield, and CO₂ fixation and the screening of step (c)(2) includes screening offspring plants by chlorophyll fluorescence to identify transgene-free plants that are putatively homozygous for the mutation. In still another embodiment of this aspect, which may be combined with any of the preceding embodiments, the target gene induces a phenotype associated with water use efficiency, and the screening of step (c)(2) includes screening offspring plants by chlorophyll fluorescence to identify transgene-free plants that are putatively homozygous for the mutation. In still another embodiment of this aspect, which may be combined with any of the preceding embodiments, the method does not include use of a plant with a hypomorphic allele or a null allele of the target gene. In a further embodiment of this aspect, which may be combined with any of the preceding embodiments, the gain of function mutation improves yield, quality, or both in the plant with the gain of function mutation as compared to a plant lacking the gain of function mutation grown under the same conditions. In an additional embodiment of this aspect, which may be combined with any of the preceding embodiments, the one or more RNA-guided nucleic acid modifying enzymes targeting the target gene include two or more different guide RNAs, three or more different guide RNAs, four or more different guide RNAs, five or more different guide RNAs, ten or more different guide RNAs, or twenty or more different guide RNAs.

In yet another embodiment of this aspect, which may be combined with any of the preceding embodiments, the guide RNAs each target a region of the target gene selected from a promoter region, an upstream regulatory region, a 5′ untranslated region (5′ UTR), a 3′ untranslated region (3′ UTR), an intron, a micro-RNA binding site, an alternative splicing element, and a downstream regulatory element. In a further embodiment of this aspect, the guide RNAs target the 5′ UTR of the target gene. In an additional embodiment of this aspect, which may be combined with any of the preceding embodiments, the guide RNAs target at least one region in the target gene that is at least 50% identical, at least 60% identical, at least 70% identical, at least 80% identical, at least 85% identical, at least 90% identical, or at least 95% identical across plant species. In still another embodiment of this aspect, which may be combined with any of the preceding embodiments, at least 50% of the set of mutations are in a region of the target gene selected from a promoter region, an upstream regulatory region, a 5′ UTR, a 3′ UTR, an intron, a micro-RNA binding site, an alternative splicing element, and a downstream regulatory element. In a further embodiment of this aspect, at least 50% of the set of mutations are in the 5′ UTR of the target gene. In still another embodiment of this aspect, which may be combined with any of the preceding embodiments, the gain of function mutation is a deletion, inversion, translocation, insertion, transition, transversion, or a combination thereof. In yet another embodiment of this aspect, which may be combined with any of the preceding embodiments, the gain of function mutation is an increase in transcription of the target gene, an increase in stability of a mRNA produced from the target gene, an increase in translation of a protein coding region of the mRNA, or a decrease in degradation of the mRNA, in each case as compared to a plant lacking the gain of function mutation grown under the same conditions. In an additional embodiment of this aspect, which may be combined with any of the preceding embodiments, the one or more RNA-guided nucleic acid modifying enzymes are Cas enzymes, base editors, or prime editors. In a further embodiment of this aspect, the Cas enzymes are selected from the group of Cas9, Cas12, Cas12a, Cas13, Cas14, CasX, or CasY.

In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In an additional embodiment of this aspect, the plant is a rice (Oryza sativa) plant, a corn (Zea mays) plant, or a cowpea (Vigna unguiculata) plant. In a further embodiment of this aspect, which may be combined with any of the previous embodiments, the plant is an elite line or elite strain.

In a further embodiment of this aspect which may be combined with any of the preceding embodiments, the target gene is selected from a photosystem II subunit S (PsbS) gene, a zeaxanthin epoxidase (ZEP) gene, and a violaxanthin de-epoxidase (VDE) gene. In an additional embodiment of this aspect, the screening includes assessing one or more of: a photosynthetic efficiency under fluctuating light conditions; a photoprotection efficiency under fluctuating light conditions; an increased rate of induction of non-photochemical quenching (NPQ) under fluctuating light conditions; an increased rate of relaxation of non-photochemical quenching (NPQ) under fluctuating light conditions; an improved quantum yield under fluctuating light conditions, and an improved CO₂ fixation under fluctuating light conditions. In yet another embodiment of this aspect, which may be combined with any of the preceding embodiments where the target gene is selected from PsbS, ZEP, or VDE, the target gene is the PsbS gene, and wherein the PsbS gene includes a sequence selected from the group of SEQ ID NO: 91, SEQ ID NO: 93, SEQ ID NO: 94, SEQ ID NO: 97, or SEQ ID NO: 98. In still another embodiment of this aspect, which may be combined with any of the preceding embodiments where the target gene is selected from PsbS, ZEP, or VDE, the target gene is the ZEP gene, and the ZEP gene includes SEQ ID NO: 92. In a further embodiment of this aspect, which may be combined with any of the preceding embodiments where the target gene is selected from PsbS, ZEP, or VDE, the target gene is the VDE gene, and wherein the VDE gene includes a sequence selected from the group of SEQ ID NO: 92 or SEQ ID NO: 95. In yet another embodiment of this aspect, the one or more different guide RNAs include spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90. In still another embodiment of this aspect, the one or more different guide RNAs include spacer sequences selected from SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37. In a further embodiment of this aspect, the one or more different guide RNAs include spacer sequences selected from SEQ ID NO: 25, SEQ ID NO: 26, SEQ ID NO: 27, SEQ ID NO: 28, SEQ ID NO: 29, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65. In yet another embodiment of this aspect, which may be combined with any of the preceding embodiments that have spacer sequences, the one or more different guide RNAs are introduced using a vector, and wherein the vector includes two or more gRNA scaffolds including SEQ ID NO: 9 and two or more tRNA linkers including SEQ ID NO: 10.

Some aspects of the disclosure are related to methods for producing an improved commercial crop plant or crop seed including: (a) selecting a commercial crop plant for improvement; (b) introducing the gain of function mutation identified in the method of any one the preceding embodiments into at least one cell of the commercial crop plant to generate an improved commercial crop plant cell; and (c) producing the improved commercial crop plant or crop seed from the improved commercial crop plant cell. In a further embodiment of this aspect, the commercial crop plant includes a rice (Oryza sativa) plant, a corn (Zea mays) plant, or a cowpea (Vigna unguiculata) plant.

An additional aspect of the disclosure includes an improved commercial crop plant or crop seed including a gain of function mutation in a non-coding sequence of a target gene, wherein the target gene induces a phenotype associated with one or more of photosynthetic efficiency, photoprotection efficiency, non-photochemical quenching, photosynthetic quantum yield, CO₂ fixation, and water use efficiency, and the gain of function mutation improves yield, quality, or both in the plant with the gain of function mutation as compared to a plant lacking the gain of function mutation grown under the same conditions. A further embodiment of this aspect includes the commercial crop plant including a rice (Oryza sativa) plant, a corn (Zea mays) plant, or a cowpea (Vigna unguiculata) plant. In an additional embodiment of this aspect, which may be combined with any of the preceding embodiments, the target gene is selected from a photosystem II subunit S (PsbS) gene, a zeaxanthin epoxidase (ZEP) gene, and a violaxanthin de-epoxidase (VDE) gene.

A further aspect of the disclosure includes an improved plant including an inversion in a cis-regulatory element of a PsbS gene, wherein the inversion increases PsbS gene expression. In a further embodiment of this aspect, the plant is a rice (Oryza sativa) plant. In still another embodiment of this aspect, the rice plant is a Oryza sativa ssp. japonica plant. In yet another embodiment of this aspect, which may be combined with any of the preceding embodiments, increased PsbS gene expression includes overexpression, increased expression at one or more specific times, increased expression in one or more specific tissues, increased expression at one or more developmental stages, or a combination thereof as compared to a control plant without the inversion in the cis-regulatory element of the PsbS gene. In still another embodiment of this aspect, which may be combined with any of the preceding embodiments, the inversion encompasses a portion of the 5′ UTR of the PsbS gene. In another embodiment of this aspect, which may be combined with any of the preceding embodiments, the inversion comprises between 1,000 and 500,000 nucleotides, between 2,000 and 400,000 nucleotides, between 3,000 and 300,000 nucleotides, between 1,000 and 10,000 nucleotides, between 2,000 and 8,000 nucleotides, between 3,000 and 5,000 nucleotides, or is about 4,000 nucleotides. In still another embodiment of this aspect, which may be combined with any of the preceding embodiments, the plant or a progenitor thereof was screened for the inversion in the cis-regulatory element of the PsbS gene and increased PsbS gene expression. In a further embodiment of this aspect, which may be combined with any of the preceding embodiments, the inversion in the cis-regulatory element of the PsbS gene was randomly produced in the plant or a progenitor thereof. In still another embodiment of this aspect, the inversion was randomly produced using guide RNAs. In yet a further embodiment of this aspect, the guide RNAs include spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90. In still another embodiment of this aspect, which may be combined with any of the preceding embodiments, the increased PsbS expression results in a phenotype associated with one or more of photosynthetic efficiency, photoprotection efficiency, non-photochemical quenching, photosynthetic quantum yield, CO₂ fixation, and water use efficiency. In an additional embodiment of this aspect, the phenotype is constitutive. In yet another embodiment of this aspect, which may be combined with any of the preceding embodiments, the increased PsbS gene expression includes overexpression of PsbS in the morning or overexpression that is not constitutive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show target guide RNA (gRNA) sites for CRISPR/Cas9 mutagenesis upstream of OsPsbS1 in Oryza sativa subspecies (ssp.) japonica. FIG. 1A shows target gRNA sites (triangles) distributed in distal (green) and proximal (yellow) regions upstream of OsPsbS1 and their location relative to the OsPsbS1 cis-regulatory element (CRE) containing the upstream promoter (black) and 5′ untranslated region (5′UTR, white) in Oryza sativa ssp. indica (top) and Oryza sativa ssp. japonica (bottom). An internal 2.7 kb O. sativa ssp. japonica-specific insertion is shown in red. FIG. 1B shows the number of lines (on x-axis) recovered per event (on y-axis) for twenty-three independent transformants.

FIGS. 2A-2F show high-throughput screening of NPQ and transgene segregation in edited T₁ progeny. FIG. 2A shows Mendelian segregation of cis-regulatory OsPsbS1 edits (purple) and inheritance of the hemizygous Cas9 transgene. In the T₀ population (top), plants contained mutated cis-regulatory elements (purple sections) and the transgene carrying Cas9 (represented by blue shape; Cas9 icon from Biorender). In the T₁ population (bottom), 25% of plants were expected to be homozygous for each mutated cis-regulatory element (represented by “A/A” (light blue squares) and “a/a” (light red squares)) and 25% of plants were expected to have lost the transgene carrying Cas9 (represented by gray prohibition sign). FIG. 2B shows the linear regression of maximum NPQ capacity (on y-axis) after 10 minutes of 1500 μmol m⁻² s⁻¹ blue light in PsbS knockout (0 on x-axis), heterozygous (1 on x-axis), and WT (2 on x-axis) lines (n=95, 117, and 101 biological replicates respectively). FIG. 2C shows that T₀ max NPQ (on x-axis) weakly correlated. with average max NPQ (on y-axis) of the corresponding T₁ alleles (n=78 T₀ plants, 2-4 biological replicates per T₁ population). Individuals with NPQ exceeding +2 SD of WT in the T₀ generation, T₁ generation, or both generations are shown in blue, yellow, and green respectively, FIG. 2D shows that chlorophyll fluorescence phenotyping of NPQ (on y-axis) over time (on x-axis; in light for 10 minutes, then in dark for 10 minutes) using leaf punches resolves homozygous alleles (A/A, blue triangle; a/a, red square) in 36 progeny of a single segregating T₀ parent relative to WT (n=4, green diamond). Heterozygous alleles are also resolved (A/a, magenta triangle). Data shown are means±SEM. FIG. 2E shows that addition of the plant selection antibiotic hygromycin to leaf punches phenotyped in FIG. 2D identifies sensitive individuals with inhibited Fᵥ/Fₘ (medium green) that lack the Cas9 transgene when phenotyped over almost 150 hours. The sensitive individuals can be distinguished from resistant individuals that include the Cas9 transgene (gold), and are similar to Wild-Type (olive green). FIG. 2F shows representative images of the chlorophyll fluorescence phenotyping. The top panel shows leaf punches from WT (top row) and leaf punches from T₁ lines (three bottom rows) before antibiotic treatment; the bottom panel shows leaf punches from WT (top row) and leaf punches from T₁ lines (three bottom rows) after 72 hours with antibiotics. Colors correspond to Fᵥ/Fₘ (a.u.), with blue indicating high and green indicating low.

FIGS. 3A-3B show the maximum NPQ of 120 putative homozygous alleles spanning 78 T₀ events. FIG. 3A shows the maximum NPQ (on 7-axis) of 120 unique OsPsbS1 alleles (on x-axis; n=1-4 technical replicates each) after 10 minutes at 1500 μmol m⁻² s⁻¹ light. The results were plotted for each unique allele, sorted from lowest to highest average NPQ capacity (left to right). Nipponbare WT NPQ of 64 biological replicates spanning all phenotyping experiments is shown (gray, open circles; far right) with boundaries demarcating±2 STD (red, dashed lines). Lines with all biological replicates below/above the WT boundary were binned by phenotype, from left to right: knockout (teal, circle), knockdown (dark green, square), WT-like (brown, hexagon), and overexpression (yellow, diamond). FIG. 3B provides the proportions of each phenotype observed, with color-coding as used in FIG. 3A; knockout=21.67%, knockdown=15%, WT-like=61.67%, and overexpression=1.67%.

FIGS. 4A-4J show that changes in PsbS protein abundance underlie NPQ and ΦPSII phenotypes. FIG. 4A shows the light intensity acclimation scheme to assess steady state chlorophyll fluorescence traits, beginning with 0 μE for 15 minutes, then 100 μE for 45 minutes (shown as three 15 minute blocks), 800 μE for 45 minutes (shown as three 15 minute blocks), 1500 μE for 45 minutes (shown as three 15 minute blocks), and ending with 0 μE for 15 minutes. For each light intensity, steady-state chlorophyll fluorescence values of the last 5 measurements (spanning 15 minutes) were averaged for data visualization (indicated by 5 black triangles). Measurements of initial Fᵥ/Fₘ and Fᵥ/Fₘ after high light (HL) are noted by single black triangles at the beginning and end of the schematic (n=3-4 biological replicates plotted on top of mean±1SEM). FIG. 4B shows the initial Fᵥ/Fₘ (on y-axis) of representative lines in Event2 and Event19: Event2 Nipponbare WT control (black, open circle, Event2_WT), Event2 knockout (teal, circle, 2-5_KO), Event2 strong knockdown (dark green, square, 2-1_SKD), Event2 weak knockdown (light green, triangle, 2-6_WKD), Event2 WT-like (brown, hexagon, 2-4_WT-like), Event2 overexpression (yellow, diamond, 2-4_OX), Event19 Nipponbare WT control (purple, open circle, Event19_WT), Event19-1 WT-like (magenta, hexagon, 19-1_WT-like), and Event19-1 overexpression (pink, diamond, 19-1_OX). FIG. 4C shows differences in steady-state NPQ capacity at 100, 800, and 1500 μmol m⁻² s⁻¹. The Event2 lines are shown on the left of the dotted line: Event2 Nipponbare WT control (black, open circle, Event2_WT), Event2 knockout (teal, circle, 2-5_KO), Event2 strong knockdown (dark green, square, 2-1_SKD), Event2 weak knockdown (light green, triangle, 2-6_WKD), Event2 WT-like (brown, hexagon, 2-4_WT-like), and Event2 overexpression (yellow, diamond, 2-4_OX). The Event19 lines are shown on the right of the dotted line: Event19 Nipponbare WT control (purple, open circle, Event19_WT), Event19-1 WT-like (magenta, hexagon, 19-1_WT-like), and Event19-1 overexpression (pink, diamond, 19-1_OX). FIG. 4D shows an immunoblot of OsPsbS1 of representative Event2 lines (from left to right: 2-5 KO, 2-1 SKD, 2-6 WKD, 2-4 WT-like, and 2-4 OX) compared to Event2 WT (on far left). FIG. 4E shows an immunoblot of OsPsbS1 of representative Event2 lines (from left to right: 2-1 SKD, 2-6 WKD, and 2-4 OX) compared to a differing amounts of Event2 WT protein (on far left and on far right: ⅛, ¼, ½, 1×, 2×, and 4× of 5 μg protein). FIG. 4F shows an immunoblot of OsPsbS1 of representative Event19 lines (19-1 OX and 19-1 WT-like) compared to Event19 WT (on far left). In FIGS. 4D-4F, 5 μg total protein were loaded per well unless otherwise noted, and representative blots of two technical replicates are shown. FIG. 4G shows NPQ capacity (on y-axis) at 1500 μmol m⁻² s⁻¹ plotted against OsPsbS1 band density (on x-axis) normalized to 1) Atpβ band intensity and 2) WT PsbS band intensity on the same blot (PsbS/Atpβ norm. to WT=1). Event2 and Event19 lines are shown: Event2 Nipponbare WT control (black, open circle, Event2_WT), Event2 knockout (teal, circle, 2-5_KO), Event2 strong knockdown (dark green, square, 2-1_SKD), Event2 weak knockdown (light green, triangle, 2-6_WKD), Event2 WT-like (brown, hexagon, 2-4_WT-like), Event2 overexpression (yellow, diamond, 2-4_OX), Event19 Nipponbare WT control (purple, open circle, Event19_WT), Event19-1 WT-like (magenta, hexagon, 19-1_WT-like), and Event19-1 overexpression (pink, diamond, 19-1_OX). Data were fit to a logarithmic curve using each individual data point (mean±1 SEM shown), with dashed lines constraining the 95% confidence interval. FIG. 4H shows correlation between NPQ and ΦPSII of lines with WT or higher NPQ capacity (e.g., OX lines) at all three light intensities denoted by colored circles (light pink, 100 μmol m⁻² s⁻¹; pink, 800 μmol m⁻² s⁻¹; dark pink, 1500 μmol m⁻² s⁻¹) and a fitted linear regression with dashed lines constraining the 95% confidence interval. FIG. 4I shows residual Fᵥ/Fₘ 15 minutes following the end of the increasing light intensity regime (n=3-4 biological replicates plotted on top of mean±1SEM). The Event2 lines are shown on the left of the dotted line: Event2 Nipponbare WT control (black, open circle, Event2_WT), Event2 knockout (teal, circle, 2-5_KO), Event2 strong knockdown (dark green, square, 2-1_SKD), Event2 weak knockdown (light green, triangle, 2-6_WKD), Event2 WT-like (brown, hexagon, 2-4_WT-like), and Event2 overexpression (yellow, diamond, 2-4_OX). The Event19 lines are shown on the right of the dotted line: Event19 Nipponbare WT control (purple, open circle, Event19_WT), Event19-1 WT-like (magenta, hexagon, 19-1_WT-like), and Event19-1 overexpression (pink, diamond, 19-1_OX). FIG. 4J shows a linear regression of residual Fᵥ/Fₘ against NPQ capacity at 1500 μmol m⁻² s⁻¹ of the Event2 lines: Event2 Nipponbare WT control (black, open circle, Event2_WT), Event2 knockout (teal, circle, 2-5_KO), Event2 strong knockdown (dark green, square, 2-1_SKD), Event2 weak knockdown (light green, triangle, 2-6_WKD), Event2 WT-like (brown, hexagon, 2-4_WT-like), and Event2 overexpression (yellow, diamond, 2-4_OX). For all pairwise comparisons, significance was determined by ordinary one-way ANOVA (α=0.05) using Dunnett's test for multiple comparisons against Nipponbare WT (FIGS. 4B-4C) or the respective Event2 or Event19 overexpression datasets (FIG. 4I) and is denoted by asterisks (**p≤0.01, ***p≤0.001, ****p≤0.0001).

FIGS. 5A-5E show chlorophyll fluorescence and gas exchange measurements of Event 2 lines with varying PsbS abundance. FIG. 5A shows NPQ (on y-axis) over different red light intensities in μE (on x-axis). FIG. 5B shows operating efficiency of PSII (ΦPSII; on y-axis) over different red light intensities in μE (on x-axis). FIG. 5C shows CO₂ assimilation (Aₙ(μmol m⁻² s⁻¹); on y-axis) over different red light intensities in μE (on x-axis). FIG. 5D shows stomatal conductance (g_{sw}(mol H₂O m⁻² s⁻¹); on y-axis) over different red light intensities in μE (on x-axis). In FIGS. 5A-5D, the measurements are a function of incident red light on mature flag leaves (n=4 biological replicates, data shown±1SEM), and significance was determined by two-way ANOVA (α=0.05) using Dunnett's test for multiple comparisons against the Nipponbare WT control, denoted by asterisks (**p≤0.01, ***p≤0.001, ****p<0.0001). FIG. 5E shows linear regression of iWUE (μmol CO₂ mol⁻¹ H₂O; on y-axis) (Aₙ/g_{sw}) as a function of maximum NPQ capacity (2000 uE; on x-axis) for each genotype (n=4 biological replicates, individual data points and means±1SEM shown). In FIGS. 5A-5E, genotypes are marked as follows: Nipponbare WT control (black, open circle, Nip_WT), Event2 strong knockdown (dark green, square, 2-1_SKD), Event2 weak knockdown (light green, triangle, 2-6_WKD), Event2 WT-like1 (brown, hexagon, 2-4_WT-like), Event2 WT-like2 (gray, inverted triangle, 2-7_WT-like), and Event2 overexpression (yellow, diamond, 2-4_OX).

FIGS. 6A-6D show the correlation of Q_{A} redox state (1-qL) and g_{sw} as a predictor of iWUE with varying PsbS levels. FIG. 6A shows Q_{A} redox state (1-qL; on y-axes) as a function of incident red light (on x-axis) on mature flag leaves (n=4 biological replicates, data shown±1SEM). Significance was determined by two-way ANOVA (α=0.05) using Dunnett's test for multiple comparisons against the Nipponbare WT control. FIG. 6B shows linear regression of 1-qL (on x-axis) as a function of g_{sw} (mol H₂O m⁻² s⁻¹) (on y-axis) for all genotypes (n=24 biological replicates; data shown±1SEM). FIG. 6C shows linear regression of 1-qL (on x-axis) as a function of g_{sw} (mol H₂O m⁻² s⁻¹) (on y-axis) for individual genotypes (n=4 biological replicates each) at all light steps exceeding 50 μmol m⁻² s⁻¹ (data shown±1SEM). For each genotype, the r² value is shown in a table below the plot: 2-1SKD=0.9766; 2-4OX=0.9831; 2-4WT=0.9866; 2-6WKD=0.9974; 2-7WT=0.9945; NipWT=0.9744. FIG. 6D shows slopes (on left) and y-intercepts (on right) of linear regression lines by genotype (data shown±1SEM). Pairwise significance in FIG. 6D was determined by ordinary one-way ANOVA (α=0.05) using Dunnett's test for multiple comparisons against Nipponbare WT and is denoted by asterisks (**p≤0.01, ***p≤0.001, ****p<0.0001). In FIGS. 6A, 6C, and 6D, genotypes are marked as follows: Nipponbare WT control (black, open circle, Nip_WT), Event2 strong knockdown (dark green, square, 2-1_SKD), Event2 weak knockdown (light green, triangle, 2-6_WKD), Event2 WT-like1 (brown, hexagon, 2-4_WT-like), Event2 WT-like2 (gray, inverted triangle, 2-7_WT-like), and Event2 overexpression (yellow, diamond, 2-4_OX).

FIGS. 7A-7C show that variation near and within the 5′UTR likely drives observed phenotypic variation. FIG. 7A shows three unique cis-regulatory mutants with large deletions (black junctions) at distal gRNA sites (green triangles) mapped onto the Nipponbare (ssp. japonica) promoter for lines 17-1_7 (top), 25-3_16 (middle), and 25-4_18 (bottom). Scale bar is 250 bp. FIG. 7B shows NPQ kinetics of the large distal deletion lines in FIG. 7A as follows: Event17-1_7 (brown circle), Event 25-3_16 (teal square), Event 25-4_18 (gray triangle), Nipponbare WT (green inverted triangle). NPQ (on y-axis) was measured over time (on x-axis; in light for 10 minutes, then in dark for 10 minutes). Data shown±1 SEM. FIG. 7C shows PCR-genotyped Event 2 mutations at proximal gRNA (yellow triangles at top) of lines with varying NPQ capacity shown in FIGS. 4A-4J, 5A-5E, and 6A-6D. From top to bottom: KO lines, Strong KD lines, Weak KD lines, WT-like lines, 24-5_18, and Nipponbare WT. Event 2-4_OX could not be genotyped by PCR. Mutations in the cis-regulatory promoter (black box) and 5′UTR (white box) are shown upstream of the OsPsbS1 coding sequence. Dashed lines indicate 146 bp of WT sequence not shown before the start codon. Deletions are shown in red, and insertions are shown in blue. Scale bar is 50 bp.

FIGS. 8A-8D show that long-read sequencing of two overexpression alleles identifies inversions upstream of OsPsbS1. FIG. 8A shows an increased resolution dot plot of the Chr. 1 locus (1.1 Mbp) harboring OsPsbS1 for the 2-4_OX line, with the break in continuity signifying the presence of a genomic inversion. FIG. 8B shows increased resolution of the Chr. 1 locus with the ˜254 kb inversion (Chr1:37693233-37948089) upstream of OsPsbS1 for the 2-4_OX line in the Integrative Genomics Viewer (IGV). FIG. 8C shows an increased resolution dot plot of the Chr. 1 locus (˜1.5 Mbp) harboring OsPsbS1 for the 19-1_OX line. FIG. 8D shows increased resolution of the Chr. 1 locus with the ˜3-4 kb inversion (Chr1:˜37693800-37696800) upstream of OsPsbS1 for the 19-1_OX line in the IGV. In FIGS. 8B and 8D, green boxes denote the OsPsbS1 gene (LOC_Os01g64960) (at top left in FIG. 8B, and at bottom right in FIG. 8D). Strength of the called structural variant was quantified by Sniffles (top orange arrow, quantified in red) and substantiated by head-to-head sequenced fragments (bottom orange arrow).

FIGS. 9A-9D show that the overexpression phenotype is constitutive, but OsPsbS1 expression is not. FIG. 9A shows qPCR results from one time point comparing OsPsbS1 expression in an overexpression line (2-4_OX), a wild type line (WT), and a knockout line (2-5_KO). FIG. 9B shows qPCR results from one time point comparing OsPsbS1 expression in an overexpression line (2-4_OX), a wild type line (WT), a wild-type-like line (2-4_WT-like), a weak knockdown line (2-6_WKD), a strong knockdown line (2-1_SKD), and a knockout line (2-5_KO). FIG. 9C shows the fold change of OsPsbS1 expression in a wild type line (WT) compared to an overexpression line (2-4_OX) over a time course where the plants were grown in 14 hour day length (DL). OsPsbS1 transcript level in 2-4_OX was statistically significantly higher only in the first timepoint (p<0.01). FIG. 9D shows time of day has negligible effect on NPQ in a wild type line (WT), a strong knockdown line (2-1_SKD), an overexpression line (2-4_OX), and a weak knockdown line (2-6_WKD) over a time course where the plants were grown in 14 hour day length (DL).

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Methods of Screening for Gain of Function Mutations

An aspect of the disclosure includes methods of screening for a gain of function mutation in a target gene in a plant including: (a) generating a set of mutations in a non-coding sequence (NCS) of the target gene in a population of plant cells of the plant with one or more RNA-guided nucleic acid modifying enzymes targeting the target gene including one or more different guide RNAs; (b) regenerating the population of plant cells into two or more plants that are hemizygous for the mutation generated; (c) (1) selfing the two or more plants to generate offspring plants, and (2) optionally screening offspring plants that are homozygous for the mutation for screening in section (d); and (d) screening the offspring plants from step (c) to identify a gain of function mutation. An additional embodiment of this aspect further includes: (e) selecting a plant with the gain of function mutation, and (f) sequencing the target gene to identify the gain of function mutation. In a further embodiment of this aspect, which may be combined with any of the preceding embodiments, the one or more RNA-guided nucleic acid modifying enzymes is expressed from an expression vector including a selectable marker and the screening in step (d) includes screening for plants lacking the selectable marker. In another embodiment of this aspect, which may be combined with any of the preceding embodiments, the gain of function mutation induces overexpression of the target gene. In a further embodiment of this aspect, overexpression of the target gene is in the morning. In an additional embodiment of this aspect, overexpression of the target gene is not constitutive. In still another embodiment of this aspect, the plant has a constitutive phenotype. In yet another embodiment of this aspect, which may be combined with any of the preceding embodiments, the target gene induces a phenotype associated with one or more of photosynthetic efficiency, photoprotection efficiency, non-photochemical quenching, photosynthetic quantum yield, and CO₂ fixation, and the screening of step (c)(2) includes screening offspring plants by chlorophyll fluorescence to identify transgene-free plants that are putatively homozygous for the mutation. In still another embodiment of this aspect, which may be combined with any of the preceding embodiments, the target gene induces a phenotype associated with water use efficiency, and the screening of step (c)(2) includes screening offspring plants by chlorophyll fluorescence to identify transgene-free plants that are putatively homozygous for the mutation. The chlorophyll fluorescence measurement phiPSII can be used to monitor the efficiency of photosynthesis in the light. When the one or more RNA-guided nucleic acid modifying enzymes is linked to an antibiotic resistance marker (e.g., hygromycin), antibiotic sensitivity screening (e.g., hygromycin sensitivity screening) can be used to increase the throughput of screening for segregation and/or loss of the one or more RNA-guided nucleic acid modifying enzymes. The sensitivity of this method is significantly cheaper and more effective than PCR in some plant species (e.g., rice), as PCR can be susceptible to contamination and false positives/negatives. By using this method in combination with screening for the desired phenotype, increased screening throughput and faster identification of lines homozygous for the mutation of interest is made possible. In still another embodiment of this aspect, which may be combined with any of the preceding embodiments, the method does not include use of a plant with a hypomorphic allele or a null allele of the target gene. In a further embodiment of this aspect, which may be combined with any of the preceding embodiments, the gain of function mutation improves yield, quality, or both in the plant with the gain of function mutation as compared to a plant lacking the gain of function mutation grown under the same conditions. In an additional embodiment of this aspect, which may be combined with any of the preceding embodiments, the one or more RNA-guided nucleic acid modifying enzymes targeting the target gene include two or more different guide RNAs, three or more different guide RNAs, four or more different guide RNAs, five or more different guide RNAs, ten or more different guide RNAs, or twenty or more different guide RNAs (e.g., twenty-four different guide RNAs). In a further embodiment of this aspect, which may be combined with any of the preceding embodiments, the one or more different guide RNAs may be split across multiple cassettes within a single T-DNA insertion (e.g., 3 cassettes with 8 gRNAs per cassette).

In yet another embodiment of this aspect, which may be combined with any of the preceding embodiments, the guide RNAs each target a region of the target gene selected from a promoter region, an upstream regulatory region, a 5′ untranslated region (5′ UTR), a 3′ untranslated region (3′ UTR), an intron, a micro-RNA binding site (e.g., a micro-RNA binding site in a mRNA, which could be in the coding region), an alternative splicing element, and a downstream regulatory element. In a further embodiment of this aspect, the guide RNAs target the 5′ UTR of the target gene. Research in corn suggests the 5′UTR may be the best source of natural allelic variation in protein expression (Gage et al. (2022) Variation in upstream open reading frames contributes to allelic diversity in maize protein abundance. PNAS, 119 (14) e2112516119). Further, editing a small region (e.g., 5′UTR) could provide a dynamic range in expression from strong knockdown to overexpression. In an additional embodiment of this aspect, which may be combined with any of the preceding embodiments, the guide RNAs target at least one region in the target gene that is at least 50% identical, at least 60% identical, at least 70% identical, at least 80% identical, at least 85% identical, at least 90% identical, or at least 95% identical across plant species. In still another embodiment of this aspect, which may be combined with any of the preceding embodiments, at least 50% of the set of mutations are in a region of the target gene selected from a promoter region, an upstream regulatory region, a 5′ UTR, a 3′ UTR, an intron, a micro-RNA binding site (e.g., a micro-RNA binding site in a mRNA, which could be in the coding region), an alternative splicing element, and a downstream regulatory element. In a further embodiment of this aspect, at least 50% of the set of mutations are in the 5′ UTR of the target gene. In still another embodiment of this aspect, which may be combined with any of the preceding embodiments, the gain of function mutation is a deletion, inversion, translocation, insertion, transition, transversion, or a combination thereof. In a further embodiment of this aspect, the mutation may alter one base or more than one base. In another embodiment of this aspect, the transition or transversion mutations result from changing one or more bases in the sequence. In yet another embodiment of this aspect, which may be combined with any of the preceding embodiments, the gain of function mutation is an increase in expression of the target gene, an increase in transcription of the target gene, an increase in stability of a mRNA produced from the target gene, an increase in translation of a protein coding region of the mRNA, or a decrease in degradation of the mRNA, in each case as compared to a plant lacking the gain of function mutation grown under the same conditions. In an additional embodiment of this aspect, the gain of function mutation is in the morning. In still another embodiment of this aspect, overexpression of the target gene is not constitutive (e.g., overexpression is time of day specific, developmental stage specific, tissue specific, etc.), and/or the plant has a constitutive phenotype. In an additional embodiment of this aspect, which may be combined with any of the preceding embodiments, the one or more RNA-guided nucleic acid modifying enzymes are Cas enzymes, base editors, or prime editors. In a further embodiment of this aspect, the Cas enzymes are selected from the group of Cas9, Cas12, Cas12a, Cas13, Cas14, CasX, or CasY. In another embodiment of this aspect, the base editors include a cytidine base editor or an adenine base editor (e.g., a dCas9 fusion protein or a Cas9 nickase fusion protein). In a further embodiment of this aspect, the prime editors include a catalytically impaired Cas9 endonuclease, such as Cas9 nickase. Other RNA-guided nucleic acid modifying enzymes known to one of skill in the art, including additional Cas enzyme types, may also be used in the methods of the present disclosure. It is thought that off-target effects when using, e.g. Cas9, are rare and/or non-consequential in plants. Off-target effects can be ruled out by ensuring 100% segregation of the mutation of interest with the desired phenotype, as an unlinked, off-target mutation functioning in trans would segregate independently. In some embodiments, the set of mutations are introduced with targeted edits (e.g., with prime editing), which may be more effective than, e.g., Cas9-mediated editing.

In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In an additional embodiment of this aspect, the plant is a rice (Oryza sativa) plant, a corn (Zea mays) plant, or a cowpea (Vigna unguiculata) plant. In a further embodiment of this aspect, which may be combined with any of the previous embodiments, the plant is an elite line or elite strain.

In a further embodiment of this aspect which may be combined with any of the preceding embodiments, the target gene is selected from a photosystem II subunit S (PsbS) gene, a zeaxanthin epoxidase (ZEP) gene, and a violaxanthin de-epoxidase (VDE) gene. In an additional embodiment of this aspect, the screening includes assessing one or more of: a photosynthetic efficiency under fluctuating light conditions; a photoprotection efficiency under fluctuating light conditions; an increased rate of induction of non-photochemical quenching (NPQ) under fluctuating light conditions; an increased rate of relaxation of non-photochemical quenching (NPQ) under fluctuating light conditions; an improved quantum yield under fluctuating light conditions, and an improved CO₂ fixation under fluctuating light conditions. In yet another embodiment of this aspect, which may be combined with any of the preceding embodiments where the target gene is selected from PsbS, ZEP, or VDE, the target gene is the PsbS gene, and wherein the PsbS gene includes a sequence selected from the group of SEQ ID NO: 91, SEQ ID NO: 93, SEQ ID NO: 94, SEQ ID NO: 97, or SEQ ID NO: 98. In still another embodiment of this aspect, which may be combined with any of the preceding embodiments where the target gene is selected from PsbS, ZEP, or VDE, the target gene is the ZEP gene, and the ZEP gene includes SEQ ID NO: 92. In a further embodiment of this aspect, which may be combined with any of the preceding embodiments where the target gene is selected from PsbS, ZEP, or VDE, the target gene is the VDE gene, and wherein the VDE gene includes a sequence selected from the group of SEQ ID NO: 92 or SEQ ID NO: 95. In yet another embodiment of this aspect, the one or more different guide RNAs include spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90. In still another embodiment of this aspect, the one or more different guide RNAs include spacer sequences selected from SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37. In a further embodiment of this aspect, the one or more different guide RNAs include spacer sequences selected from SEQ ID NO: 25, SEQ ID NO: 26, SEQ ID NO: 27, SEQ ID NO: 28, SEQ ID NO: 29, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65. In yet another embodiment of this aspect, which may be combined with any of the preceding embodiments that have spacer sequences, the one or more different guide RNAs are introduced using a vector, and wherein the vector includes two or more gRNA scaffolds including SEQ ID NO: 9 and two or more tRNA linkers including SEQ ID NO: 10.

### Methods of Producing Improved Commercial Crop Plants or Crop Seeds and Improved Plants

Some aspects of the disclosure are related to methods for producing an improved commercial crop plant or crop seed including: (a) selecting a commercial crop plant for improvement; (b) introducing the gain of function mutation identified in the method of any one the preceding embodiments into at least one cell of the commercial crop plant to generate an improved commercial crop plant cell; and (c) producing the improved commercial crop plant or crop seed from the improved commercial crop plant cell. In a further embodiment of this aspect, the commercial crop plant includes a rice (Oryza sativa) plant, a corn (Zea mays) plant, or a cowpea (Vigna unguiculata) plant.

An additional aspect of the disclosure includes an improved commercial crop plant or crop seed including a gain of function mutation in a non-coding sequence of a target gene, wherein the target gene induces a phenotype associated with one or more of photosynthetic efficiency, photoprotection efficiency, non-photochemical quenching, photosynthetic quantum yield, CO₂ fixation, and water use efficiency, and wherein the gain of function mutation improves yield, quality, or both in the plant with the gain of function mutation as compared to a plant lacking the gain of function mutation grown under the same conditions. A further embodiment of this aspect includes the commercial crop plant including a rice (Oryza sativa) plant, a corn (Zea mays) plant, or a cowpea (Vigna unguiculata) plant. In an additional embodiment of this aspect, which may be combined with any of the preceding embodiments, the target gene is selected from a photosystem II subunit S (PsbS) gene, a zeaxanthin epoxidase (ZEP) gene, and a violaxanthin de-epoxidase (VDE) gene. Further aspects of the disclosure are related to plant parts, tissues, or cells of the plants of any of the above embodiments.

A further aspect of the disclosure includes an improved plant including an inversion in a cis-regulatory element of a PsbS gene, wherein the inversion increases PsbS gene expression. In a further embodiment of this aspect, the plant is a rice (Oryza sativa) plant. In still another embodiment of this aspect, the rice plant is a Oryza sativa ssp. japonica plant. In yet another embodiment of this aspect, which may be combined with any of the preceding embodiments, increased PsbS gene expression includes overexpression, increased expression at one or more specific times, increased expression in one or more specific tissues, increased expression at one or more developmental stages, or a combination thereof as compared to a control plant without the inversion in the cis-regulatory element of the PsbS gene. In still another embodiment of this aspect, which may be combined with any of the preceding embodiments, the inversion encompasses a portion of the 5′ UTR of the PsbS gene. In another embodiment of this aspect, which may be combined with any of the preceding embodiments, the inversion comprises between 1,000 and 500,000 nucleotides, between 2,000 and 400,000 nucleotides, between 3,000 and 300,000 nucleotides, between 1,000 and 10,000 nucleotides, between 2,000 and 8,000 nucleotides, between 3,000 and 5,000 nucleotides, or is about 4,000 nucleotides. In still another embodiment of this aspect, which may be combined with any of the preceding embodiments, the plant or a progenitor thereof was screened for the inversion in the cis-regulatory element of the PsbS gene and increased PsbS gene expression. In a further embodiment of this aspect, which may be combined with any of the preceding embodiments, the inversion in the cis-regulatory element of the PsbS gene was randomly produced in the plant or a progenitor thereof. In still another embodiment of this aspect, the inversion was randomly produced using guide RNAs. In yet a further embodiment of this aspect, the guide RNAs include spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90. In still another embodiment of this aspect, which may be combined with any of the preceding embodiments, the increased PsbS expression results in a phenotype associated with one or more of photosynthetic efficiency, photoprotection efficiency, non-photochemical quenching, photosynthetic quantum yield, CO₂ fixation, and water use efficiency. In a further embodiment of this aspect, the phenotype is constitutive. In an additional embodiment of this aspect, which may be combined with any of the preceding embodiments, the increased PsbS gene expression includes overexpression of PsbS in the morning or overexpression that is not constitutive.

A “control plant” as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

The term “plant” is used in its broadest sense. It includes, but is not limited to, any species of woody, ornamental or decorative, crop or cereal, fruit or vegetable plant, and algae (e.g., Chlamydomonas reinhardtii). It also refers to a plurality of plant cells that is largely differentiated into a structure that is present at any stage of a plant's development. Such structures include, but are not limited to, a fruit, shoot, stem, leaf, flower petal, etc.

The term “plant tissue” includes differentiated and undifferentiated tissues of plants including those present in roots, shoots, leaves, inflorescences, anthers, pollen, ovaries, seeds and tumors, as well as cells in culture (e.g., single cells, protoplasts, embryos, callus, etc.). Plant tissue may be in planta, in organ culture, tissue culture, or cell culture.

The term “plant part” as used herein refers to a plant structure, a plant organ, or a plant tissue. In certain embodiments, the plant part may be a seed, pod, fruit, leaf, flower, stem, root, any part of the foregoing or a cell thereof, or a non-regenerable part or cell of a genetically modified or improved plant part. As used in this context, a “non-regenerable” part or cell of a genetically modified or improved plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

The term “plant cell” refers to a structural and physiological unit of a plant, comprising a protoplast and a cell wall. The plant cell may be in the form of an isolated single cell or a cultured cell, or as a part of a higher organized unit such as, for example, a plant tissue, a plant organ, or a whole plant. The term “plant cell culture” refers to cultures of plant units such as, for example, protoplasts, cells and cell clusters in a liquid medium or on a solid medium, cells in plant tissues and organs, microspores and pollen, pollen tubes, anthers, ovules, embryo sacs, zygotes and embryos at various stages of development.

The term “plant material” refers to leaves, stems, roots, inflorescences and flowers or flower parts, fruits, pollen, anthers, egg cells, zygotes, seeds, cuttings, cell or tissue cultures, or any other part or product of a plant.

A “plant organ” refers to a distinct and visibly structured and differentiated part of a plant, such as a root, stem, leaf, flower bud, inflorescence, spikelet, floret, seed or embryo.

The term “crop plant”, means in particular monocotyledons such as cereals (wheat, millet, sorghum, rye, triticale, oats, barley, teff, spelt, buckwheat, fonio and quinoa), rice, maize (corn), and/or sugar cane; or dicotyledon crops such as beet (such as sugar beet or fodder beet); fruits (such as pomes, stone fruits or soft fruits, for example apples, pears, plums, peaches, almonds, cherries, strawberries, raspberries or blackberries); leguminous plants (such as beans, lentils, peas or soybeans); oil plants (such as rape, mustard, poppy, olives, sunflowers, coconut, castor oil plants, cocoa beans or groundnuts); cucumber plants (such as marrows, cucumbers or melons); fiber plants (such as cotton, flax, hemp or jute); citrus fruit (such as oranges, lemons, grapefruit or mandarins); vegetables (such as spinach, lettuce, cabbages, carrots, tomatoes, potatoes, cucurbits or paprika); lauraceae (such as avocados, cinnamon or camphor); tobacco; nuts; coffee; tea; vines; hops; durian; bananas; natural rubber plants; and ornamentals (such as flowers, shrubs, broad-leaved trees or evergreens, for example conifers). This list does not represent any limitation.

The term “woody crop” or “woody plant” means a plant that produces wood as its structural tissue. Woody crops include trees, shrubs, or lianas. Examples of woody crops include, but are not limited to, thornless locust, hybrid chestnut, black walnut, Japanese maple, eucalyptus, casuarina, spruce, fir, pine, and flowering dogwood.

The term “improved growth” or “increased growth” is used herein in its broadest sense. It includes any improvement or enhancement in the process of plant growth and development. Examples of improved growth include, but are not limited to, increased photosynthetic efficiency, increased biomass, increased yield, increased seed number, increased seed weight, increased stem height, increased leaf area, increased root biomass, and increased plant dry weight,

The term “quantum yield” refers to the moles of CO₂ fixed per mole of quanta (photons) absorbed, or else the efficiency with which light is used to convert CO₂ into fixed carbon. The quantum yield of photosynthesis is derived from measurements of light intensity and rate of photosynthesis. As such, the quantum yield is a measure of the efficiency with which absorbed light produces a particular effect. The amount of photosynthesis performed in a plant cell or plant can be indirectly detected by measuring the amount of starch produced by the transgenic plant or plant cell. The amount of photosynthesis in a plant cell culture or a plant can also be detected using a CO₂ detector (e.g., a decrease or consumption of CO₂ indicates an increased level of photosynthesis) or an O₂ detector (e.g., an increase in the levels of O₂ indicates an increased level of photosynthesis (see, e.g., the methods described in Silva et al., Aquatic Biology 7:127-141, 2009; and Bai et al., Biotechnol. Lett.33:1675-1681, 2011). Photosynthesis can also be measured using radioactively labeled CO₂ (e.g., 14CO₂ and H₁₄CO₃⁻) (see, e.g., the methods described in Silva et al., Aquatic Biology 7:127-141, 2009, and the references cited therein). Photosynthesis can also be measured by detecting the chlorophyll fluorescence (e.g., Silva et al., Aquatic Biology 7:127-141, 2009, and the references cited therein). Additional methods for detecting photosynthesis in a plant are described in Zhang et al., Mol. Biol. Rep.38:4369-4379, 2011.

In the physical sciences, the term “relaxation” means the return of a perturbed system into equilibrium, usually from a high energy level to a low energy level. As used herein, the term “non-photochemical quenching relaxation” or “NPQ relaxation” refers to the process in which NPQ level decreases upon transition from high light intensity to low light intensity.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

### Molecular Biological Methods to Produce Transgenic Plants, Plant Parts, and Plant Cells

One aspect of the present disclosure provides transgenic plants, plant parts, or plant cells including gain of function mutations in non-coding sequences of target genes including photosystem II subunit S (PsbS), zeaxanthin epoxidase (ZEP), and violaxanthin de-epoxidase (VDE). In addition, the present disclosure provides guide RNA (gRNA) spacer sequences, gRNA scaffold sequences, and tRNA linker sequences that may be used to screen and/or generate mutations in non-coding sequences of target genes in a population of plant cells.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al.. The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (KAY et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466), the maize ubiquitin promoter (CHRISTENSEN & QUAIL, Transgenic Res, 5, 213-8, 1996), the trefoil promoter (Ljubql, MAEKAWA et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al.. The Plant J (1992) 2, 834-844), the emu promoter (Last et al.. Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067) , the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in green tissues (such as the promoter of the chlorophyll a/b binding protein (Cab)). The plant Cab promoter (Mitra et al., Planta, (2009) 5: 1015-1022) has been described to be a strong bidirectional promoter for expression in green tissue (e.g., leaves and stems) and is useful in one embodiment of the current disclosure. These plant-expressible promoters can be combined with enhancer elements, they can be combined with minimal promoter elements, or can include repeated elements to ensure the expression profile desired.

Additional non-limiting examples of tissue-specific promoters include the maize allothioneine promoter (DE FRAMOND et al, FEBS 290, 103-106, 1991; Application EP 452269), the chitinase promoter (SAMAC et al. Plant Physiol 93, 907-914, 1990), the maize ZRP2 promoter (U.S. Pat. No. 5,633,363), the tomato LeExtl promoter (Bucher et al. Plant Physiol. 128, 911-923, 2002), the glutamine synthetase soybean root promoter (HIREL et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), and the Arabidopsis pCO₂ promoter (HEIDSTRA et al, Genes Dev. 18, 1964-1969, 2004). Further non-limiting examples of tissue-specific promoters include the RbcS2B promoter, RbcS1B promoter, RbcS3B promoter, LHB1B1 promoter, LHB1B2 promoter, cab1 promoter, and other promoters described in Engler et al., ACS Synthetic Biology, DOI: 10.1021/sb4001504, 2014. These plant promoters can be combined with enhancer elements, they can be combined with minimal promoter elements, or can include repeated elements to ensure the expression profile desired.

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Therms aquaticus, the amplification process can be completely automated. Other enzymes which can be used are known to those skilled in the art.

Nucleic acids and proteins of the present disclosure can also encompass homologs of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch—3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: Editing the Rice Photosystem H Subunit S(OsPsbS1) Gene Using CRISPR/Cas9

This example describes a high-throughput pipeline for screening novel alleles generated by CRISPR/Cas9 non-coding sequence mutagenesis upstream of OsPsbS1. The findings presented in this example reveal the ability to generate a large phenotypic range of activity, including overexpression, with significant effects on NPQ and iWUE. Finally, the results identified themes in cis-regulation across the allelic library that may inform gene editing for overexpression within other traits.

### Materials and Methods

### Plant Growth Conditions

Rice cultivar Nipponbare (Oryza sativa ssp. japonica) seeds were germinated on Whatman filter paper for seven days at 100 μmol m⁻² s⁻¹ fluorescent light with a 14-hour day length (27° C. day/25° C. night temperature). Seedlings were transferred to soil composed of equal parts Turface and Sunshine Mix #4 (Sungro) and grown under seasonal day-length (10-14 hours) in a south-facing greenhouse that fluctuated in temperature (38° C. High/16° C. Low) and relative humidity (45-60%). Plants were fertilized with a 0.1% Sprint 330 iron supplement after transplanting at 2 weeks post-germination, and at the onset of grain filling at 10 weeks post germination. In addition, plants were fertilized with JR Peter's Blue 20-20-20 fertilizer monthly. Flats were kept full of water to mimic flooded growth conditions. Genotypes were randomized across flats and throughout the greenhouse to minimize positional effects. At the V4-5 leaf stage, T₁ progeny and WT controls were assayed for differences in NPQ capacity and sensitivity to the selectable marker hygromycin.

To assess differences in photosynthetic efficiency and yield, gene-edited T₂ plants were grown in a larger greenhouse with more homogenous light exposure and field-relevant growth conditions (40° C. High/27° C. Low, 30-50% relative humidity) without supplemental light. Plants were grown and yield data were collected concurrent to the California rice growing season (April-October), after which the plants were dried down for 2 weeks. Above-ground dry biomass, yield, and 1000 seed weight were quantified by destructive harvest.

### Construct Cloning and Guide RNA Design

Eight guide RNA (gRNA) target sites were identified upstream of the functional PsbS ortholog in rice, OsPsbS1 (LOC_Os01g64960), using CRISPR-P (crispr[dot]hzau[dot]edu[dot]cn) and a 1.5-kb region upstream of the OsPsbS1 start codon in a draft genome of Oryza sativa ssp. indica cultivar IR64 (Schatz et al., 2014, schatzlab[dot]cshl[dot]edu/data/rice/). The eight gRNA spacers were then assembled into a DNA cassette interspersed with scaffolds and tRNA linkers for polycistronic gRNA expression as previously described (Xie K, Minkenberg B, Yang Y. Boosting CRISPR/Cas9 multiplex editing capability with the endogenous tRNA-processing system. Proc Natl Acad Sci. 2015;112(11):3570-3575. doi:10.1073/pnas.1420294112), and synthesized (Genscript). The insert was cloned into the pRGEB32 rice Agrobacterium-mediated transformation vector (Addgene Plasmid #63142) via GoldenGate Assembly to produce the pRGEB32_OsPsbS1_8xgRNA vector. In this vector, an OsU3 promoter drove expression of the polycistronic gRNA-tRNA cassette with scaffolds, a ZmUbi promoter drove expression of a dual nuclear-localized SpCas9 codon-optimized for rice, and a hygromycin resistance gene (HygR) was used for Agrobacterium-mediated transformation of embryogenic rice calli. Sequences are provided in Table 1, below.

TABLE 1
OsPsbS1 non-coding sequence gRNA spacer sequences and
positions relative to the start codon

| | | Position from ATG | Position from ATG | |
|---|---|---|---|---|
| | Orientation | in O. sativa ssp. | in O. sativa ssp. | |
| | (rel. to | indica cultivar | japonica cultivar | Spacer Sequence |
| Insert | ORF) | IR64 | Nipponbare | (5′ -> 3′) |
| gRNA1 | R | -1163:-1183 | -3837:-3857 | GCGAGACACTAAAATACATT |
| | | | | (SEQ ID NO: 1) |
| gRNA2 | F | -973:-953 | -3647:-3627 | TCTTGTTCCTGGATGTAATT |
| | | | | (SEQ ID NO: 2) |
| gRNA3 | R | -888:-908 | -3562:-3582 | AGATTCAGGAGTAACAAAAA |
| | | | | (SEQ ID NO: 3) |
| gRNA4 | F | -769:-749 | -344:-3423 | TGTTGCATGTGGTCCGTCGA |
| | | | | (SEQ ID NO: 4) |
| gRNA5 | R | -585:-605 | -3259:-3279 | CACAAAAAAGTACGGGAAT |
| | | | | G (SEQ ID NO: 5) |
| gRNA6 | F | -374:-394 | -374:-394 | TACCAACCACCTGCTCTTCT |
| | | | | (SEQ ID NO: 6) |
| gRNA7 | R | -263:-283 | -263:-283 | GTCTCCCCGAATCCCTTCTA |
| | | | | (SEQ ID NO: 7) |
| gRNA8 | F | -166:-146 | -166:-146 | CTACGCCTCCCACCCGCCAC |
| | | | | (SEQ ID NO: 8) |

| | |
|---|---|
| gRNA | GTTTTAGAGCTAGAAATAGCAAGTTAAAATAAGGCTAGTCCGTTATCAACTTGAAAAAGT |
| scaffold | GGCACCGAGTCGGTGC (SEQ ID NO: 9) |
| tRNA | AACAAAGCACCAGTGGTCTAGTGGTAGAATAGTACCCTGCCACGGTACAGACCCGGGTTC |
| linker | GATTCCCGGCTGGTGCA (SEQ ID NO: 10) |

### In Vitro gRNA Validation of Upstream OsPsbS1 Non-Coding Sequences

Each candidate gRNA spacer was synthesized as a ssDNA oligomer with additional 5′ and 3′ overhangs necessary for T7 RNA Polymerase transcription and binding of the gRNA scaffold primer respectively (Table 2). Oligomers were synthesized into dsDNA via Phusion™ High-Fidelity PCR (NEB) and transcribed into RNA using the HiScribe RNA synthesis kit (NEB). Residual dsDNA was digested via a DNAseI treatment, and RNA was purified using the RNeasy mini-kit (Qiagen). Cas9 was individually complexed with each gRNA spacer in a buffer of 2 mM HEPES, 10 mM NaCl, 0.5 mM MgCl₂, 10 μm EDTA, pH 6.5) for 20 minutes at 37° C. before co-incubating the complexed ribonucleoprotein with 100 ng of the PCR-amplified, 2-kb region upstream of OsPsbS1 (ssp. indica) overnight at 37° C. to verify activity of all eight guides (Error! Reference source not found.).

TABLE 2
Oligomers for in vitro Cas9-gRNA editing of
O. sativa (ssp. indica) OsPsbs1 non-coding
sequences

| | |
|---|---|
| 5′ overhang | TAATACGACTCACTATAGGG (SEQ ID NO: 99) |
| 3′ overhang | GTTTAAGAGCTATGCTGGAA (SEQ ID NO: 100) |
| gRNA scaffolding | AAAAGCACCGACTCGGTGCCACTTTTTCAAGTTGATAACGGACTAGCCTTATTT |
| primer | AAACTTGCTATGCTGTTTCCAGCATAGCTCTTAAAC (SEQ ID NO: 101) |
| EXAMPLE: | TAATACGACTCACTATAGGG-[GCGAGACACTAAAATACATT]- |
| OsPsbS1 gRNA1 | GTTTAAGAGCTATGCTGGAA (SEQ ID NO: 102) |
| oDP211 | CTACCTATGCAACATGTGACCC (SEQ ID NO: 103) |
| OsPsbS1 NCS F | |
| oDP214 | AGACAGAGGTATGTCAATGTGTTATTG (SEQ ID NO: 11) |
| OsPsbS1 NCS R | |

### Induction of Embryogenic Calli

Mature seeds of rice (Oryza sativa ssp. japonica cv. Nipponbare) were de-hulled, and surface-sterilized for 20 min in 20% (v/v) commercial bleach (5.25% sodium hypochlorite) plus a drop of Tween 20. Three washes in sterile water were used to remove residual bleach from seeds. De-hulled seeds were placed on callus induction medium (CIM) medium (N6 salts and vitamins (Chu, C. C. et al. Establishment of an efficient medium for anther culture of rice, through comparative experiments on the nitrogen sources. Sci Sin 18, (1975)), 30 g/L maltose, 0.1 g/L myo-inositol, 0.3 g/L casein enzymatic hydrolysate, 0.5 g/L L-proline, 0.5 g/L L-glutamine, 2.5 mg/L 2,4-D, 0.2 mg/L BAP, 5 mM CuSO₄, 3.5 g/L Phytagel, pH 5.8) and incubated in the dark at 28° C. to initiate callus induction. Six- to eight-week-old embryogenic calli were used as targets for transformation.

### Agrobacterium-Mediated Transformation

Embryogenic calli were dried for 30 min prior to incubation with an Agrobacterium tumefaciens EHA105 suspension (OD_{600 nm}=0.1) carrying the cloned binary vector, pRGEB32_OsPsbS1_8xgRNA. After a 30 min incubation, the Agrobacterium suspension was removed. Calli were then placed on sterile filter paper, transferred to co-cultivation medium (N6 salts and vitamins, 30 g/L maltose, 10 g/L glucose, 0.1 g/L myo-inositol, 0.3 g/L casein enzymatic hydrolysate, 0.5 g/L L-proline, 0.5 g/L L-glutamine, 2 mg/L 2,4-D, 0.5 mg/L thiamine, 100 mM acetosyringone, 3.5 g/L Phytagel, pH 5.2) and incubated in the dark at 21° C. for 3 days. After co-cultivation, calli were transferred to resting medium (N6 salts and vitamins, 30 g/L maltose, 0.1 g/L myo-inositol, 0.3 g/L casein enzymatic hydrolysate, 0.5 g/L L-proline, 0.5 g/L L-glutamine, 2 mg/L 2,4-D, 0.5 mg/L thiamine, 100 mg/L timentin, 3.5 g/L Phytagel, pH 5.8) and incubated in the dark at 28° C. for 7 days. Calli were then transferred to selection medium (CIM plus 250 mg/L cefotaxime and 50 mg/L hygromycin B) and allowed to proliferate in the dark at 28° C. for 14 days. Well-proliferating tissues were transferred to CIM containing 75 mg/L hygromycin B.

The remaining tissues were subcultured at 3- to 4-week intervals on fresh selection medium. When a sufficient amount (about 1.5 cm in diameter) of the putatively transformed tissues was obtained, they were transferred to regeneration medium (MS salts and vitamins (Murashige, T. & Skoog, F. A Revised Medium for Rapid Growth and Bio Assays with Tobacco Tissue Cultures. Physiologia Plantarum 15, (1962)), 30 g/L sucrose, 30 g/L sorbitol, 0.5 mg/L NAA, 1 mg/L BAP, 150 mg/L cefotaxime) containing 40 mg/L hygromycin B and incubated at 26 ° C., 16-hr light, 90 μmol photons m⁻² s⁻¹. When regenerated plantlets reached at least 1 cm in height, they were transferred to rooting medium (MS salts and vitamins, 20 g/L sucrose, 1 g/L myo-inositol, 150 mg/L cefotaxime) containing 20 mg/L hygromycin B and incubated at 26° C. under conditions of 16-hour light (150 μmol photons m⁻² s⁻¹) and 8-hour dark until roots were established and leaves touched the Phytatray lid. When possible, multiple regenerants per calli were recovered. Plantlets were then transferred to soil.

### Chlorophyll Fluorescence Measurements of NPQ

Leaf punches were sampled from mature, fully developed leaves at leaf stage V3-5 and floated on 270 μL of water in a 96-well plate. Plates were dark acclimated for at least 30 minutes prior to analysis. In vivo chlorophyll fluorescence measurements were determined at room temperature using an Imaging-PAM Maxi (Walz) pulse-amplitude modulation fluorometer. Fluorescence levels after dark acclimation (F₀, Fₘ) and during light acclimation (F₀′, Fₘ′) were monitored were monitored in two ways:

To resolve phenotypically segregating OsPsbS1 NCS gene-edited alleles from a single parent, a single leaf punch at the V3 stage was exposed to a 4-minute period of high-intensity actinic light (1500 μmol m⁻² s⁻¹) using periodic saturated pulses. Putative homozygous lines were identified as those progeny in the top and bottom 10% of total NPQ. The leaf punches were then used to determine hygromycin sensitivity and Cas9 transgene segregation as described.

Candidates determined to lack Cas9 were re-sampled at the V5 leaf stage, phenotyping two leaf punches from two mature leaves per plant to compare NPQ relative to Nipponbare WT plants. NPQ was quantified during a 10-minute period of high-intensity actinic light (1500 μmol m⁻² s⁻¹) and 10 minutes dark relaxation (0 μmol m⁻² s⁻¹) using periodic saturating pulses. NPQ in both cases was calculated using the below formula.

NPQ=[(Fₘ−Fₘ′)/Fₘ′] (1)

### Hygromycin Sensitivity Assay for High-Throughput Cas9 Transgene Detection

Floated leaf punches assayed for NPQ capacity were subsequently used to determine hygromycin sensitivity and segregation of the transgene. Hygromycin B (50 mg/mL 1×PBS) was added to each well to reach a final concentration of 20 μg/mL of antibiotic. Plates were incubated under rice germination chamber conditions for three days, following which the Imaging-PAM Maxi (Walz) was used to identify differences in the maximum efficiency of Photosystem II (Fᵥ/Fₘ) after 30 minutes of dark acclimation, calculated using the below formula.

Fᵥ/Fₘ=(Fₘ−Fₒ)/(Fₘ) (2)

Sensitive, transgene-free plants typically had a decline in Fᵥ/Fₘ of >0.3-0.4, whereas transgenic plants maintained a WT Fᵥ/Fₘ of ˜0.7-0.8.

### Total RNA/Protein Extraction of Representative Lines with Varying NPQ

2 cm of leaf tissue from the youngest fully developed leaf was collected and flash-frozen in RNAse-free, DNAse-free tubes containing Lysing Matrix D (FastPrep-24™) at midday. Leaf tissue was ground on dry ice using a FastPrep-24 5G™High-Speed Homogenizer (6.0 m/s for 2×40 s, MP Biomedical). Protein and mRNA were extracted from the same leaf sample (NucleoSpin RNA/Protein kit, REF740933, Macherey-Nagel GmbH & Co., Duren, Germany).

### Quantitative RT-PCR of OsPsbS1 Relative to Two Reference Genes

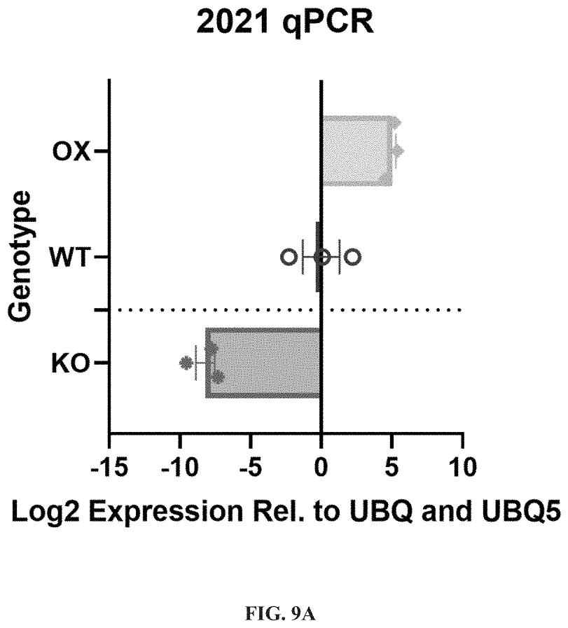

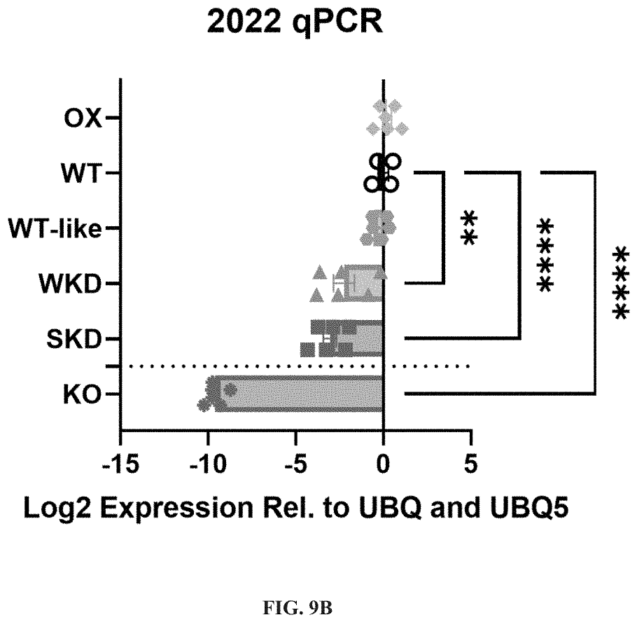

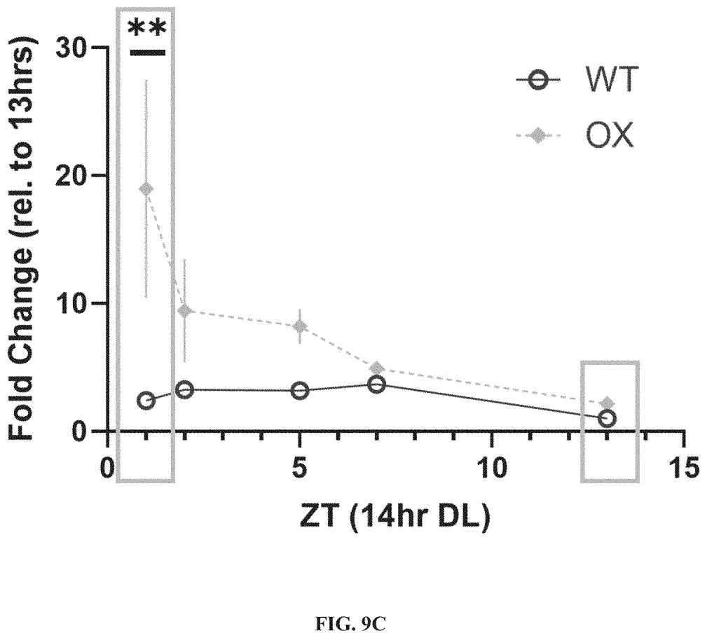

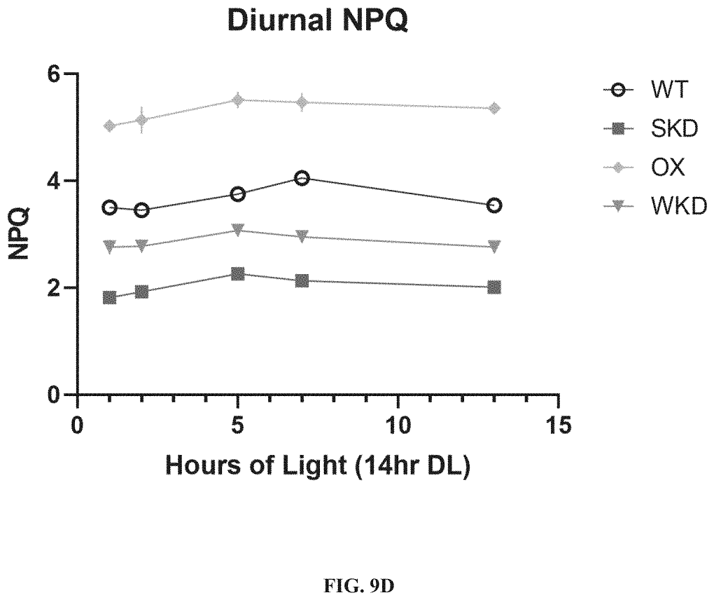

Extracted mRNA was treated with DNase (ThermoFisher Scientific) and transcribed to cDNA using Omniscript Reverse Transcriptase (Qiagen) and a 1:1 mixture of random hexamers and oligo dT as recommended by the manufacturer. Quantitative reverse transcription PCR was used to quantify OsPsbS1 transcripts relative to OsUBQ and OsUBQ5 transcripts, in biological triplicate with technical duplicates using published methods to normalize qRT-PCR expression to multiple reference genes (Vandesompele, J. et al. Accurate normalization of real-time quantitative RT-PCR data by geometric averaging of multiple internal control genes. Genome Biol 3, (2002); Hellemans, J., Mortier, G., De Paepe, A., Speleman, F. & Vandesompele, J. qBase relative quantification framework and software for management and automated analysis of real-time quantitative PCR data. Genome Biol 8, R19 (2007)). Samples were run on a 7500 Fast Real-Time PCR system (Applied Biosystems, Gent, Belgium) in a total volume of 20 μL using 4 μL of 1:10 diluted cDNA. Primers were empirically validated on a 5-step dilution series of WT cDNA (1:1 to 1:81). All final primer pairs had an amplification efficiency between 90-105% and linear amplification within the dynamic range tested. A single peak in melt-curve analysis was observed for each gene of interest, verifying specificity of the amplicon. UBQ and PsbS1 were described in Fu, X. et al. The coordination of OsbZIP72 and OsMYBS2 with reverse roles regulates the transcription of OsPsbS1 in rice. New Phytol 229, 370-387 (2021), and UBQ5 was described in Jain, M., Nijhawan, A., Tyagi, A. K. & Khurana, J. P. Validation of housekeeping genes as internal control for studying gene expression in rice by quantitative real-time PCR. Biochemical and Biophysical Research Communications 345, 646-651 (2006). Transcript levels were measured in greenhouse-grown plants in the morning (FIG. 9A), the evening (FIG. 9B), and during a time-course of growth chamber-grown plants (FIG. 9C). Primers and primer efficiencies are shown in Table 3, below.

TABLE 3
qRT-PCR primer pairs and observed primer efficiency in WT
across a 1:81 dilution series.

| | | | Primer |
|---|---|---|---|
| Gene ID | Primer Name | Sequence | Efficiency |
| UBQ | oDP578q UBQ-F_Fu2020 | TGCACCCTAGGGCTGTCAAC | 95.3% |
| LOC_Os03g13170 | | (SEQ ID NO: 17) | |
| | oDP579q UBQ-R-Fu2020 | GGCGAGTGACGCTCTAGTTCTT | |
| | | (SEQ ID NO: 18) | |
| UBQ5 | oDP580q UBQ5- | ACCACTTCGACCGCCACTACT | 105.1% |
| LOC_Os01g22490 | F_Jain2006 | (SEQ ID NO: 19) | |
| | oDP581q UBQ5- | ACGCCTAAGCCTGCTGGTT | |
| | R_Jain2006 | (SEQ ID NO: 20) | |
| PsbS1 | oDP584q PsbS1F-Fu2020 | CTGTTCGGCAGGTCCAAG | 95.9% |
| LOC_Os01g64960 | | (SEQ ID NO: 23) | |
| | oDP586q PsbS1R2-DP | CAAACCCGAGCATGGCGA | |
| | | (SEQ ID NO: 24) | |

### Immunoblotting of Whole Leaf Protein Extracts

Precipitated protein was resuspended in the supplied protein solubilization buffer (PSB-TCEP) and quantified using a TCA colorimetric assay (Karlsson, J. O., Ostwald, K., Kabjorn, C. & Andersson, M. A Method for Protein Assay in Laemmli Buffer. Analytical Biochemistry 219, 144-146 (1994)). Samples containing 5 μg total protein were resolved using pre-cast SDS-PAGE Any KD™ gels (BIO-RAD), transferred to a polyvinylidene difluoride membrane (Immobilon-FL 0.45 μm, Millipore) via wet transfer, and blocked with 3% nonfat dry milk for immunodetection. Membranes were cut and incubated with the following antibodies. A rabbit polyclonal antibody raised against sorghum PsbS (SbPsbS) was generously shared by Steven J. Burgess (University of Illinois) and used at a 1:2,500 dilution. A rabbit polyclonal antibody raised against a synthetic peptide of the β-subunit of ATP synthase (Atpβ) was obtained from Agrisera (catalogue no. AS05 085) and used at 1:10,000 dilution. After incubation with an HRP-conjugated, anti-rabbit secondary antibody from GE Healthcare (1:10,000 dilution), bands were detected by chemiluminescence using SuperSignal West Femto Maximum Sensitivity Substrate (Thermo Scientific). Protein bands were quantified by densitometry with ImageQuant TL software (version 7.0 GE Healthcare Life Sciences, Pittsburgh, PA, USA). PsbS abundance was quantified relative to WT based on a dilution series of WT PsbS protein.

### Steady-State NPQ Acclimation to Low, Moderate, and High Light

To assess steady-state differences in NPQ capacity across representative lines, genotypes varying in PsbS abundance were grown and the youngest fully expanded leaf of 8-week-old vegetative rice plants was phenotyped. Whole plants were dark acclimated for 1 hour and phenotyped at the following regime, quantifying chlorophyll fluorescence by a multiphase flash routine (L16800, LI-COR, Lincoln, NE, USA) every 3 minutes: 15 min 0 μmol m⁻² s⁻¹-45 min 100 μmol m⁻² s⁻¹-45 min 800 μmol m⁻² s⁻¹-45 min 1500 μmol m⁻² s⁻¹-15 min 0 μmol m⁻² s⁻¹, using increasing red-light intensity and a total of 10 μmol m⁻² s⁻¹ blue-light for all light-dependent measurements. In addition to formulas 1 and 2 (above), operating efficiency of PSII (ΦPSII) was also monitored using the below formula:

ΦPSII=(Fₘ′−Fₛ′)/Fₘ′ (3)

### In Parallel Gas Exchange and Chlorophyll Fluorescence Analysis Under Increasing Red Light

Photosynthetic gas exchange dynamics were measured on the youngest, fully expanded flag leaf of 12-week-old flowering rice plants. Gas exchange measurements were performed using an open gas exchange system (L16800, LI-COR, Lincoln, NE, USA) equipped with a 2-cm² leaf chamber and integrated modulated fluorometer. Whole plants were low light acclimated for 1-2 hours to mitigate afternoon depression of photosynthesis and dark acclimated for at least 30 minutes to allow for concurrent phenotyping of gas exchange (e.g. Aₙ, g_{sw}) and chlorophyll fluorescence parameters (e.g. Fᵥ/Fₘ, NPQ). For all measurements the chamber conditions were set to: 400 ppm chamber [CO₂], 27° C. chamber temperature, 1.4 kPa vapor pressure deficit of the leaf, 500 μmol s⁻¹ flow rate, and a fan speed of 10,000 rpm. Samples were assayed within the boundaries of ambient daylength (8 am-5 pm).

Steady state photosynthesis and stomatal conductance was monitored in response to changes in red light intensity (100% red LED's, λₚₑₐₖ 630 nm). Light intensity was varied from 0, 50, 80, 110, 140, 170, 200, 300, 400, 600, 800, 1000, 1500, and 2000 μmol m⁻² s⁻¹ with 10-20 minutes of acclimation per light step. Steady state was reached when the stomatal conductance, g_{sw}, maintained a slope less than 0.005 +/−0.00025 SD over a 40 second period and when net assilimation rate, Aₙ, showed variation less than 0.5 +/−0.25 SD over a 20 s period. Net assimilation rate, stomatal conductance, and intracellular [CO2] was logged. A saturating pulse was then applied to collect all relevant chlorophyll fluorescence parameters using a multiphase flash routine. In addition to NPQ and Fᵥ/Fₘ, intrinsic water use efficiency (iWUE) (formula 4, below), ΦPSII (formula 2, above), and Q_{A} redox state (formula 5, below) were assessed. The derivation of 1-qL assumes a “lake” model for photosynthetic antenna complexes.

iWUE=Aₙ/g_{sw} (4)

1-qL =1−(F_{q}′/Fᵥ′)/(Fₒ/Fₛ′) (5)

### PCR genotyping of transgene free, edited lines

50 mg of leaf tissue was ground via bead beating (Lysing Matrix D, FastPrep-24™) and genomic DNA was extracted in 2xCTAB buffer at 65° C. for 15 minutes. DNA was separated via chloroform phase separation and precipitated using isopropanol. The pellet was washed briefly in 70% ethanol before being dried and resuspended in 1×TE buffer.

Two overlapping regions spanning the 8 gRNA target sites were PCR-amplified via Phusion™ High-Fidelity PCR using 5× GC-rich buffer (Table 4). PCR products were amplified from at least two putative homozygotes per line and sequenced by Sanger Sequencing. Contigs of the ˜4.3-kb upstream region were assembled using Snapgene.

TABLE 4
Primers used for PCR amplification and genotyping of OsPsbS1 NCS.

| | Forward Primer | Reverse Primer | Amplicon | |
|---|---|---|---|---|
| Amplicon | (5′ -> 3′) | (5′ -> 3′) | Size | Add. Seq Primer |
| Distal end | AGACAGAGGTATGT | AAAGAGCAAATGGC | 2384 bp | CGTGTCTCCCA |
| (gRNA 1-5) | CAATGTGTTATTG | CTACCA (SEQ ID NO: | | CGTCTTCTT |
| | (SEQ ID NO: 11) | 13) | | (SEQ ID NO: 15) |
| Proximal end | TGGTAGGCCATTTG | CATCCATCCAAATTC | 2167 bp | GAGCAAACAC |
| (gRNA 6-8) | CTCTTT | CAACCT (SEQ ID NO: | | TCAGGCACAA |
| | (SEQ ID NO: 12) | 14) | | (SEQ ID NO: 16) |

### Pacbio Long-Read Whole-Genome Sequencing and Analysis

Leaf tissue used for high-molecular weight genomic DNA (HMW gDNA) extraction was dark starved for 4 days before being sampled and flash frozen in liquid nitrogen. DNA was isolated using the NucleoBond HMW DNA kit (TakaraBio, Catalog #740160.2) with the following modifications: Plant leaves were ground by pestle and mortar under liquid nitrogen, where 1 g of ground leaf tissue was resuspended in 2.5 times the amount of recommended lysis buffer and incubated in a 50° C. water bath for 4 hours. The amount of Binding Buffer H2 was also proportionately increased. HMW gDNA was resuspended by gentle pipetting in water and assessed for quality by Femto Pulse Analysis (median fragment length 13-15 kb).

HMW gDNA was pooled and sequenced using a PacBio Sequel II (QB3 Genomics, UC Berkeley, Berkeley, CA, RRID:SCR_022170) by HiFi circular consensus sequencing (CCS) on a single 8M SMRTcell. Untrimmed reads for overexpression lines N2-4_OX and N19-1_OX were first quality checked using FastQC (www[dot]bioinformatics[dot]babraham[dot]ac[dot]uk/projects/fastqc/). Reads were mapped to the Oryza sativa v7.0 reference genome (Ouyang, S. et al. The TIGR Rice Genome Annotation Resource: improvements and new features. Nucleic Acids Research 35, D883-D887 (2007)) using pbmm2, a minimap2 wrapper by Pacific Biosciences specifically for HiFi reads and assembled using pbIPA, Pacific Biosciences phased assembler (github[dot]com/PacificBiosciences/pbbioconda). Sniffles (github[dot]com/fritzsedlazeck/Sniffles) and pbsv (github[dot]com/PacificBiosciences/pbbioconda) were used to identify structural variants from mapped reads. D-Genies (D-GENIES: dot plot large genomes in an interactive, efficient and simple way [PeeJ]. Peerj[dot]com/articles/4958/) was used to align the de novo assembly to the reference genome and generate dot plots. Integrative Genomics Viewer (Robinson, J. T. et al. Integrative genomics viewer. Nat Biotechnol 29, 24-26 (2011)) was used to visualize mapped reads, aligned assemblies, and sniffles structural variation output.

### Results

### A Library of Independent Events for Multiplexed Editing Upstream of OsPsbS1

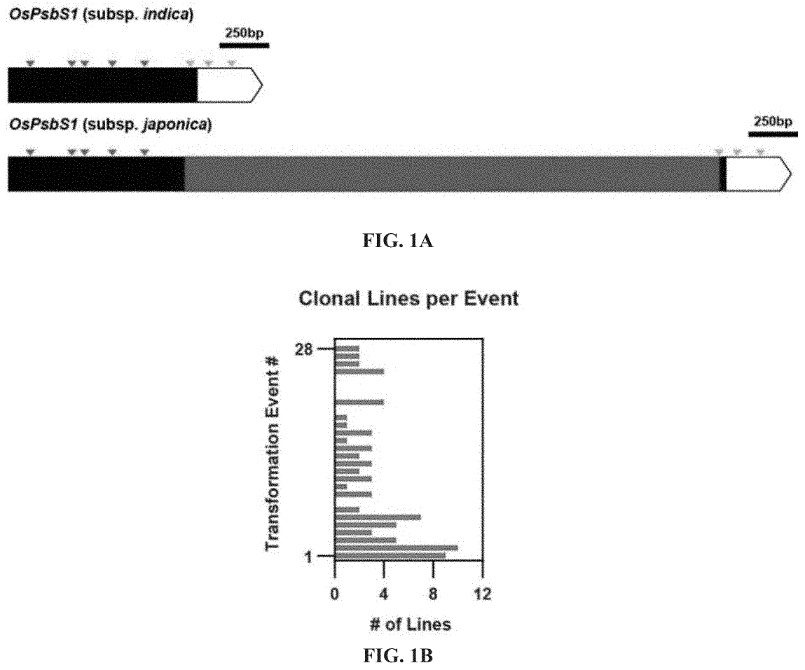

To generate novel cis-regulatory variation upstream of OsPsbS1 (LOC_Os01g64960), eight specific and evolutionarily conserved guide RNA (gRNA) target sites (Table 1) were introduced into rice (Oryza sativa ssp. japonica) cultivar Nipponbare calli via Agrobacterium-mediated transformation. The design avoided targeting a putative QTL for NPQ activity in rice upstream of OsPsbS1, an internal 2.7 kb japonica-specific insertion which had previously been identified (Kasajima, I. et al. Molecular distinction in genetic regulation of nonphotochemical quenching in rice. Proceedings of the National Academy of Sciences 108, 13835-13840 (2011); Wang Q, Zhao H, Jiang J, et al. Genetic Architecture of Natural Variation in Rice Nonphotochemical Quenching Capacity Revealed by Genome-Wide Association Study. 2017;8 (October). doi:10.3389/fpls.2017.01773) (FIG. 1A). Twenty-three fertile, independent transformants were generated (FIG. 1B). Multiple sister lines were recovered from the same transformed callus when possible, yielding 78 T₀ plants. Clonal lines that were recovered from the same transformation event were differentiated by hyphens, with the number before the hyphen corresponding to the event number and the number after the hyphen corresponding to the line number (e.g., for event #2, clonal lines would be designated 2-1, 2-2, 2-3, etc.).

### High-Throughput Chlorophyll Fluorescence Screening of Edited, Semi-Dominant PsbS Alleles

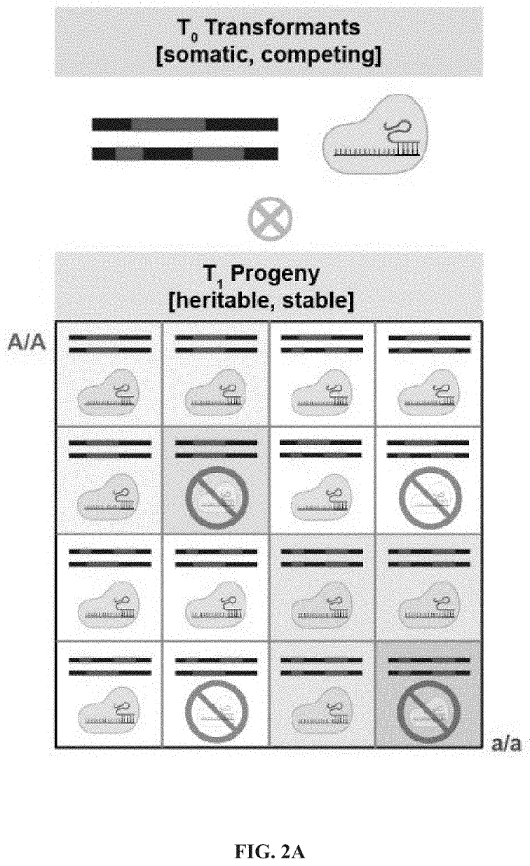

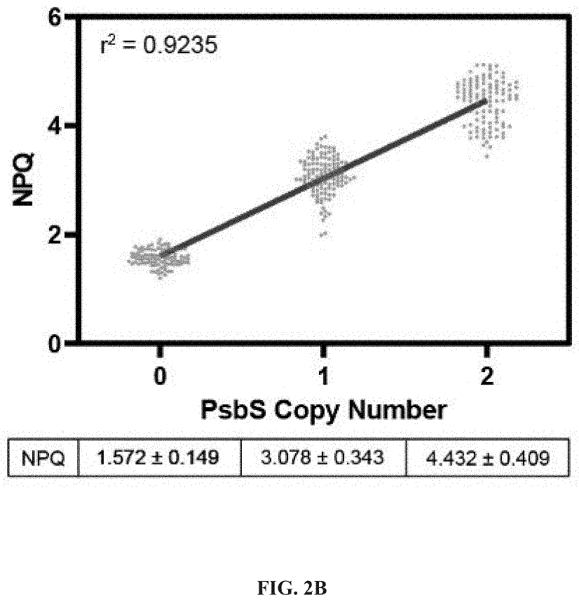

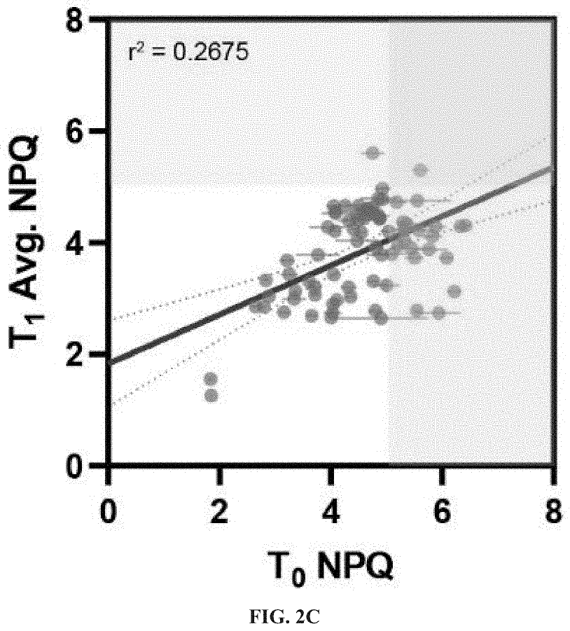

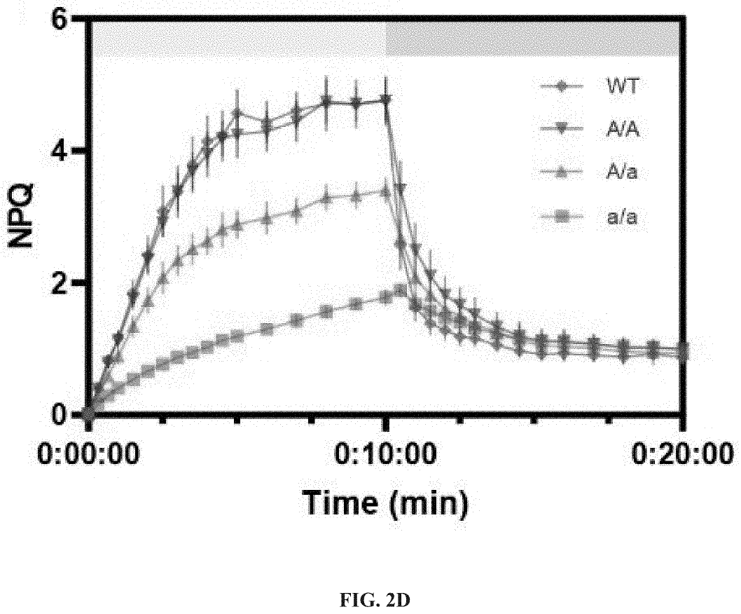

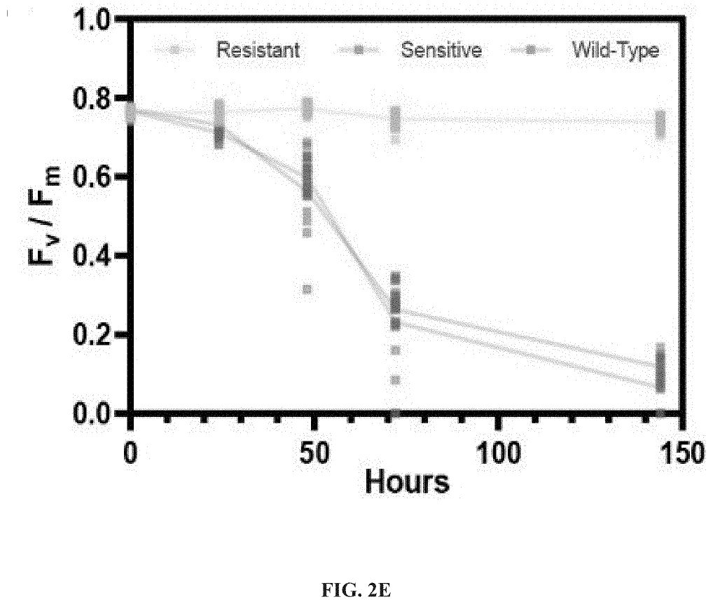

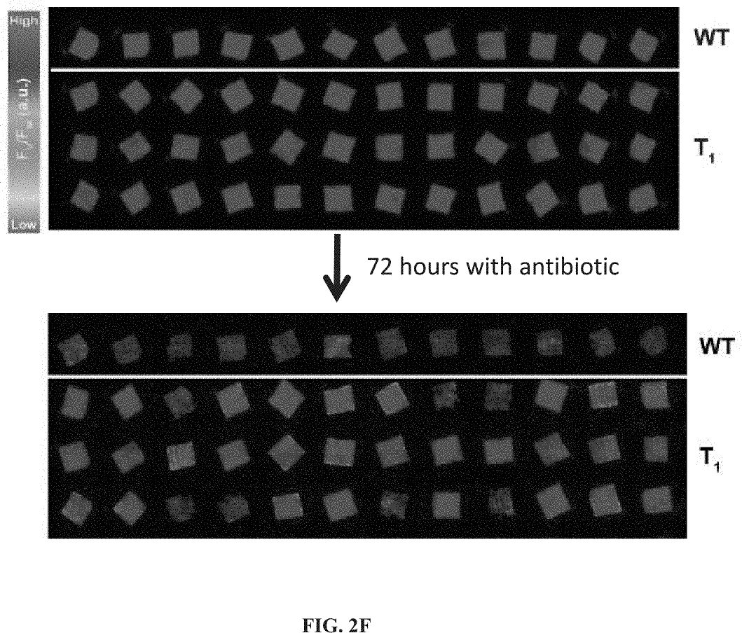

In vivo chlorophyll fluorescence screening was used to resolve unique phenotypes across the 78 diploid T₀ transfortnants, yielding up to 156 gene-edited alleles. However, it was known that phenotypes in the T₀ generation may be somatic and competition between alleles may mask dynamic changes in gene expression and activity. To circumvent this, each transformant was screened in the T₁ generation where alleles were fixed to homozygosity and the hemizygous Cas9 transgene could be segregated away. Based on Mendelian segregation of heritable alleles and single insertion of the transgene, ¼ of T₁ progeny (resulting from selfing of the T₀ line) were expected to be homozygous for each mutated cis-regulatory element and ¼ of progeny were expected to have lost the transgene carrying Cas9 and the plant antibiotic resistance cassette (FIG. 2A), Relying on the fact that CRISPR/Cas9 will largely produce biallelic mutations, the T₁ progeny were screened for differences in phenotype between the two alleles (designated A and a) that may help to identify progeny that are heterozygous [A/a] or homozygous for either OsPsbS1 allele [A/A or a/a].

PsbS is a gene in which loss-of-function mutations exhibit semi-dominance. These mutations showed a strong linear correlation between copy number and NPQ capacity (r²=0.9235) (FIG. 2B). Despite this, T₀ max NPQ was a poor predictor of the average of T₁ fitness (r²=0.2675). T₀ phenotypes overestimated the number of putative T₁ overexpressors (blue) and failed to identify a candidate stable overexpressor (yellow) (FIG. 2C). To resolve stable, heritable alleles, populations of T₁ progeny were screened at high throughput to determine differences in NPQ capacity and Cas9 transgene segregation. Putative homozygous alleles were identified phenotypically (A/A and a/a, FIG. 2D), with more subtle differences in population phenotypes resolved by binning the upper and lower 10% of NPQ phenotypes. Leaf punches used to assess NPQ capacity were treated with the plant selection antibiotic hygromycin and monitored for a decline in the quantum efficiency of photosystem II (Fᵥ/Fₘ), a common indicator of plant stress and photodamage (FIGS. 2E-2F). Obvious differences could be observed after only 72 hours of incubation, with a decrease in Fᵥ/Fₘ of over 75%. Using this approach, homozygous, Cas9-free plants could be identified after <2 hours of work, allowing the rapid identification of fixed germplasm to move into the T₂ generation.

### Gene-Edited, Overexpression Alleles Are Present, Albeit Rare

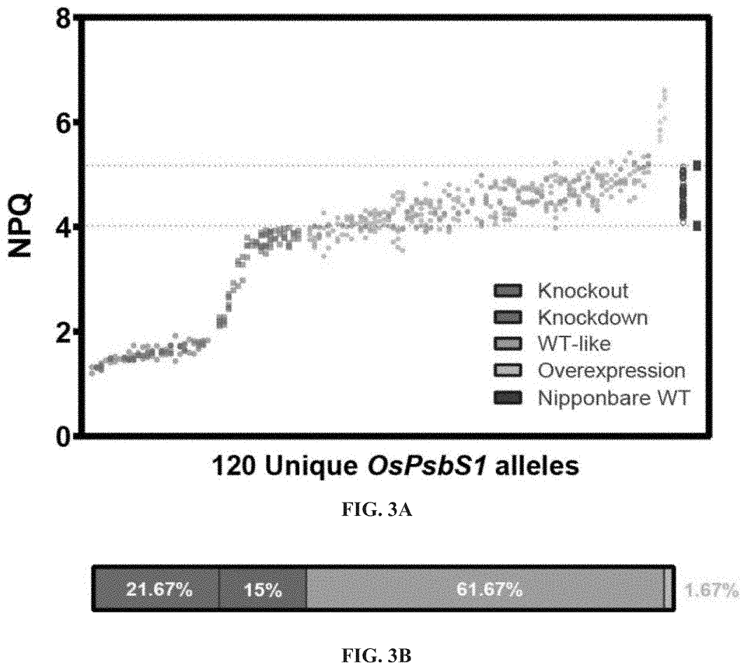

Putative homozygous alleles were identified via pairwise comparison between WT plants and progeny from a single T₀ parent, as depicted in (FIG. 2D). To assess the variation in phenotypes across all putative stable alleles, maximum NPQ for all 120 phenotypically resolved alleles was plotted (FIG. 3A),

Almost two-thirds of the 120 stable alleles isolated were WT-like in NPQ capacity (61.67%), with the second and third largest groups being knockout (21.67%) and knockdown (15%) phenotypes, respectively. Two independent overexpression alleles were isolated, comprising 1.67% of the total phenotypic variation (FIG. 3B).

### Chlorophyll Fluorescence Phenotypes Correlate with PsbS Protein Abundance

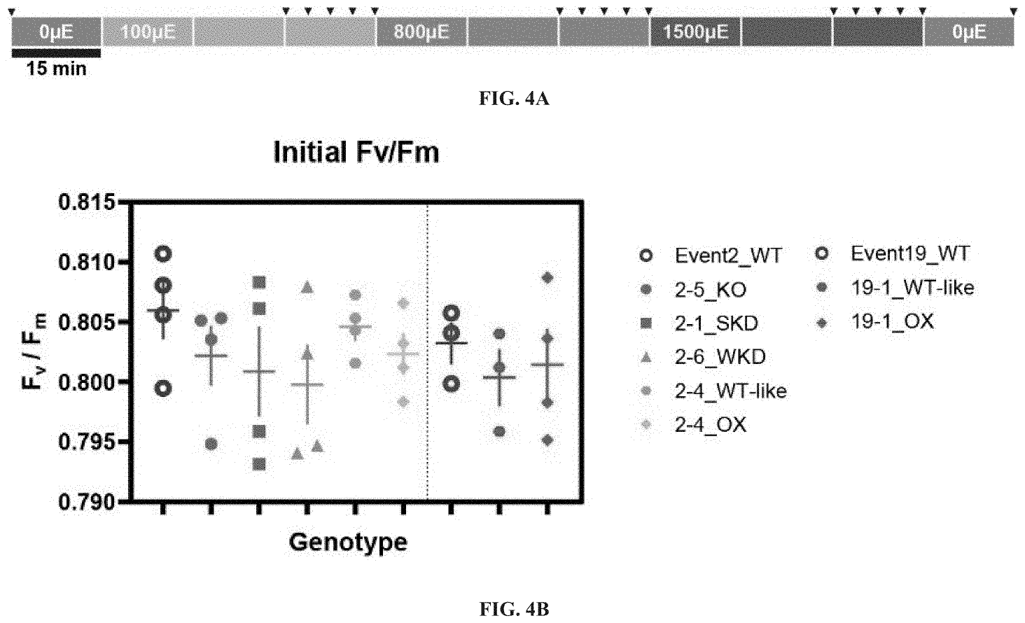

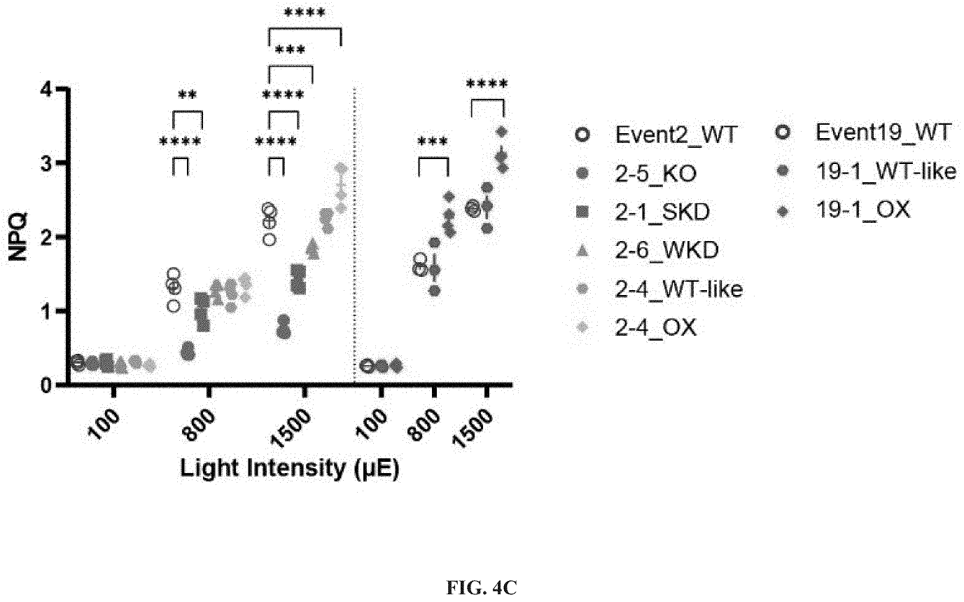

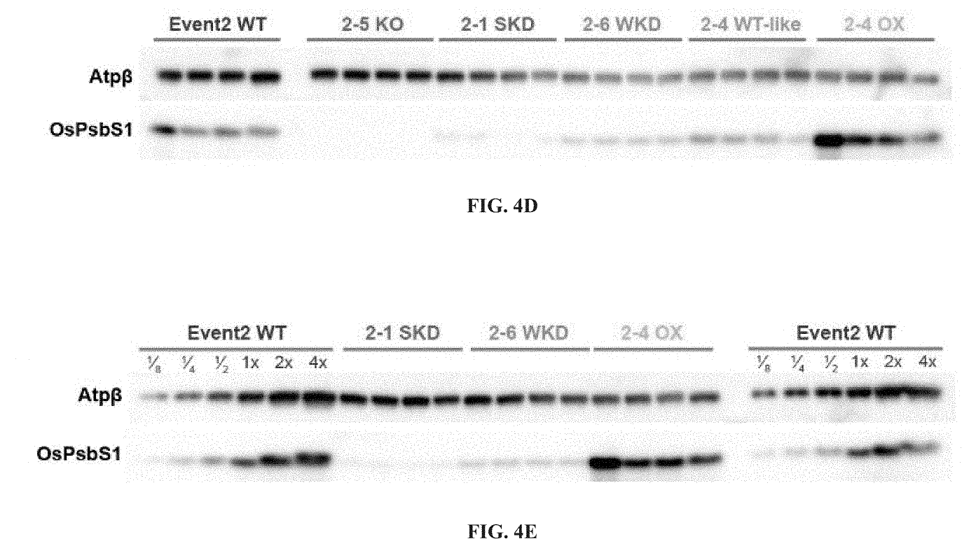

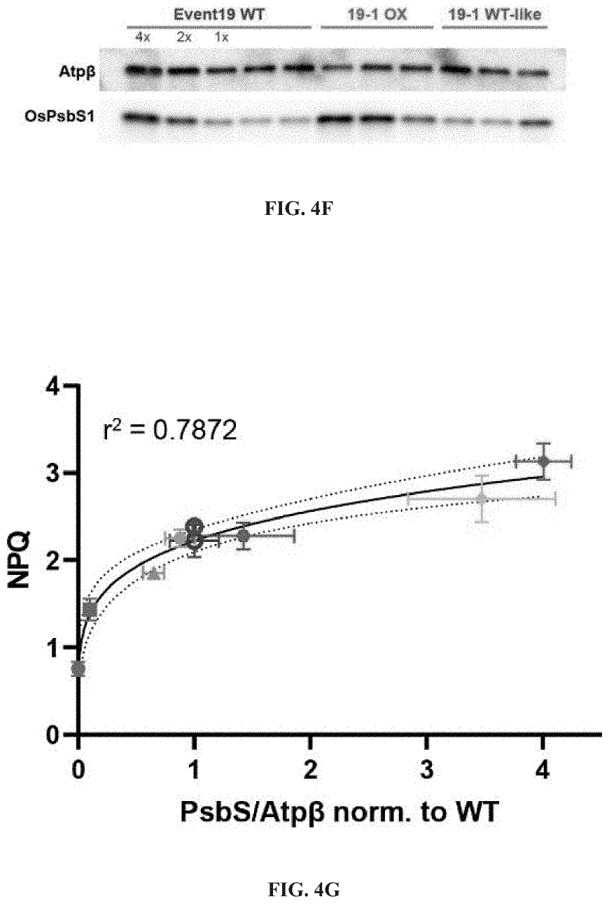

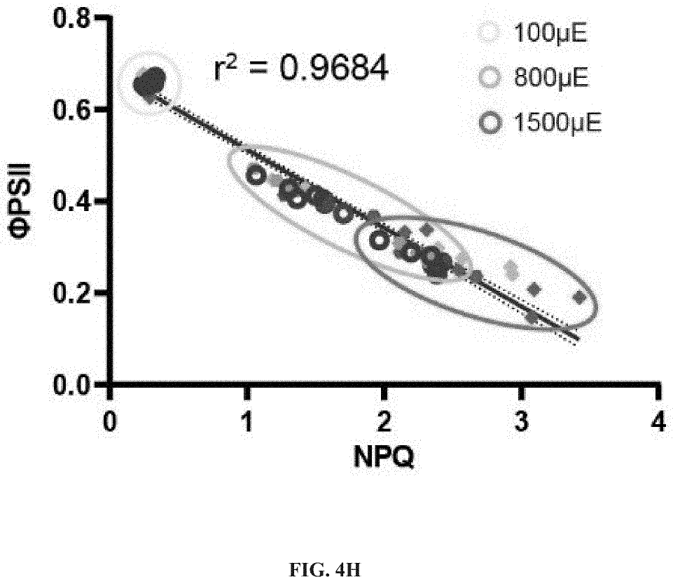

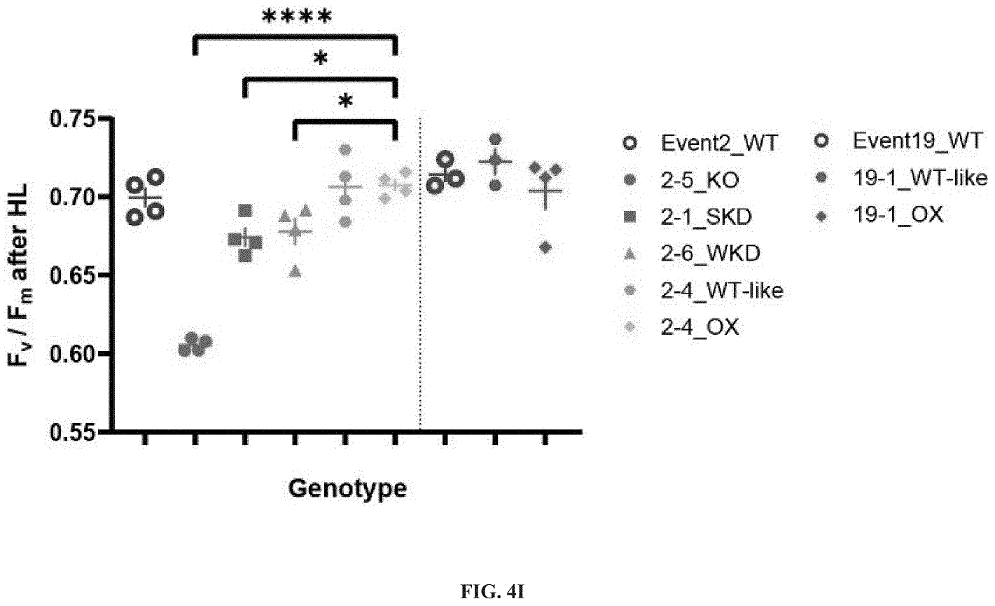

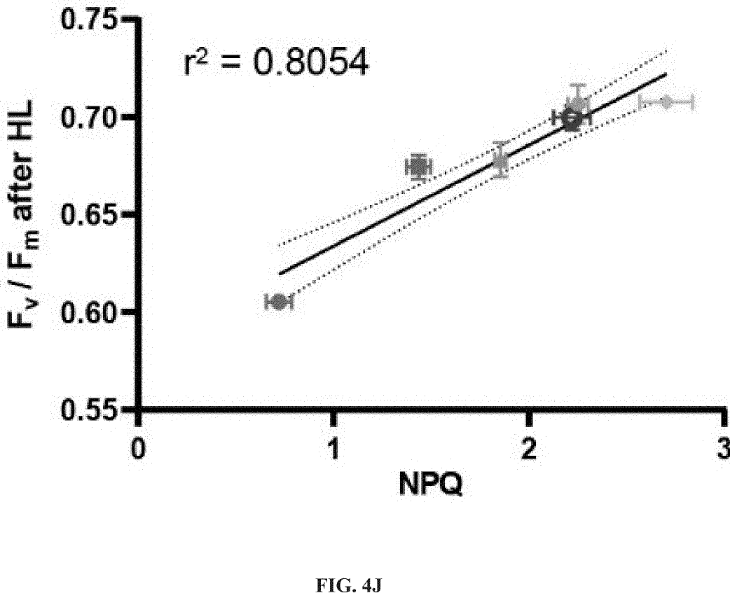

To better understand the phenotypic variation generated, representative lines from the two events that yielded overexpression alleles, Event2 and Event19, were assessed for their ability to acclimate to low, moderate, and high light (FIG. 4A). Assayed individuals showed no differences in initial Fᵥ/Fₘ (FIG. 4B) or NPQ at 100 μmol m−2 s−1 (FIG. 4C). At 800 μmol m−2 s−1 of light, the Event2-5 knockout line (2-5_KO) and Event2-1 strong knockdown (2-1_SKD) line showed significantly reduced NPQ capacity relative to the Event2 WT control (p<0,0001 and p=0.0041 respectively). In contrast, the Event19-1 overexpression line (19-1_OX) showed significantly higher NPQ capacity than the Event 19 WT control under the same condition (p=0.0001). At 1500 μmol m−2 s−1, significant differences between all non-WT-like alleles were observed (p<0.0003) (FIG. 4C).

Immunoblot analysis of the OsPsbS1 protein highlighted the dynamic range of absolute protein abundance (FIG. 4D) and abundance relative to a Nipponbare WT control dilution series (FIGS. 4E-4F). In Event2, the 2-1_SKD allele and 2-6 weak knockdown allele (2-6_WKD) accumulated ⅛ and ¼ of the WT OsPsbS1 protein level, respectively. In contrast, the 2-4 overexpression allele (2-4_OX) accumulated 2-4 times the WT OsPsbS1 protein, similar to 19-1_OX. 2-5_KO was below the threshold of PsbS detection. When normalized against the loading control, chloroplastic Atpβ, as well as the Nipponbare WT controls of each membrane to allow comparison across experiments, it was possible to fit a logarithmic relationship between OsPsbS1 protein abundance and NPQ capacity at 1500 μmol m⁻² s⁻¹ for all genotypes (r²=0.7872), consistent with PsbS dosage-dependent activity observed previously in A. thaliana¹¹⁹ (FIG. 4G).

NPQ is photoprotective but can compete with light harvesting. To assess the extent to which photosynthesis was constrained in the isolated overexpression alleles, ΦPSII, the operating efficiency of PSII in the light, was plotted against NPQ at all three light conditions for WT and OX lines (FIG. 4H). A strong negative correlation was observed (r² =0.9684), with individuals with the highest NPQ (i.e. 19-1_OX) showing a lower ΦPSII at both 800 μmol m⁻² s⁻¹ and 1500 μmol m⁻² s⁻¹ relative to other genotypes at that light intensity. To determine the extent to which NPQ was photoprotective, Fᵥ/Fₘ was measured 15 minutes after the final light step of the assayed light regime. Relative to 2-4_OX, the 2-5_KO, 2-1_SKD, and 2-6_WKD alleles showed greater amounts of photoinhibition as indicated by declines in Fᵥ/Fₘ (p<0.0001, p=0.011, p=0.024 respectively) (FIG. 4I). There was no difference in phenotype between the overexpression and WT-like alleles for each event. However, plotting Fᵥ/Fₘ after the light regime against NPQ capacity at the last 1500 μmol m⁻² s⁻¹ last step revealed a strong correlation between NPQ capacity and the maintenance of the quantum efficiency of PSII (FIG. 4J). Thus steady-state acclimation of rice lines at varying PsbS abundances and light intensities revealed possible tradeoffs between light harvesting and photoprotection at varying PsbS levels.

### Varying PsbS Abundance Alters Steady State Gas Exchange Phenotypes in Response to Red Light

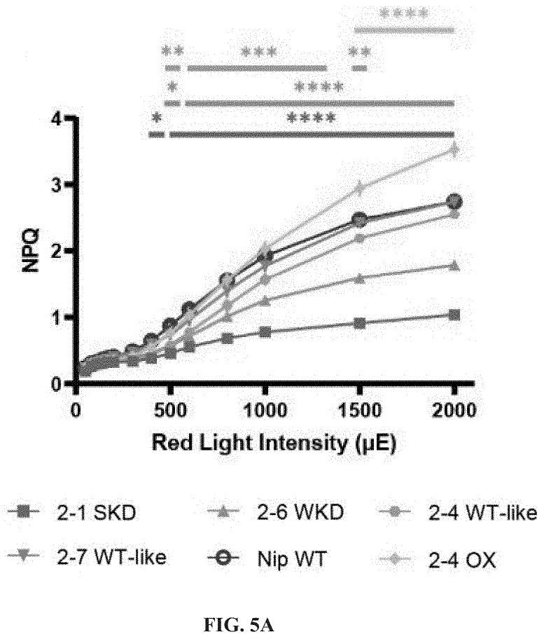

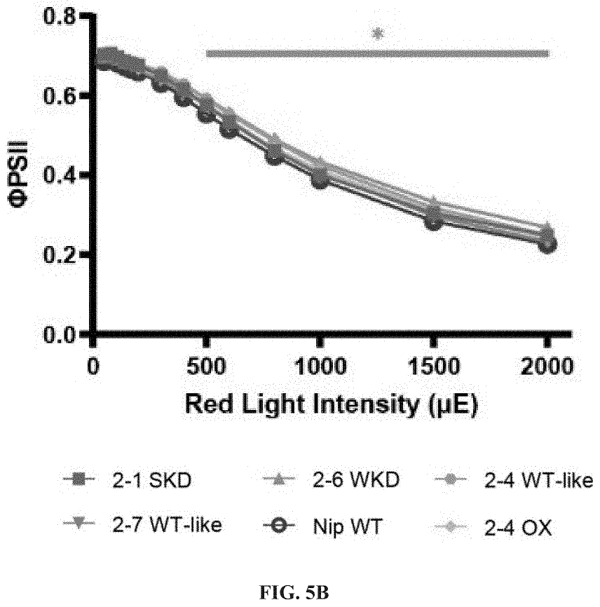

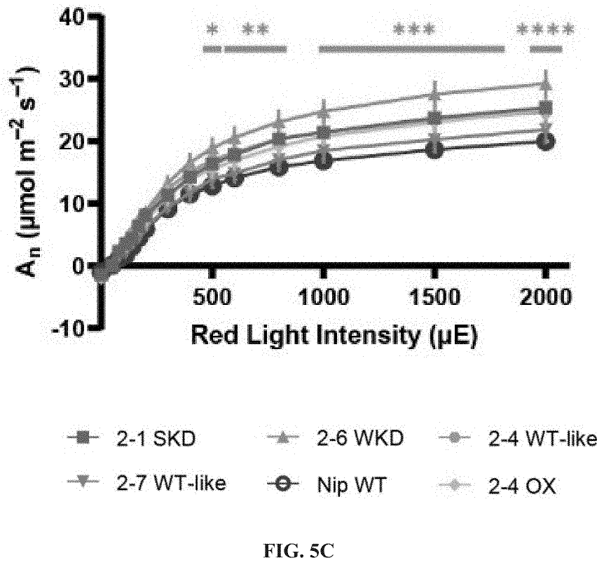

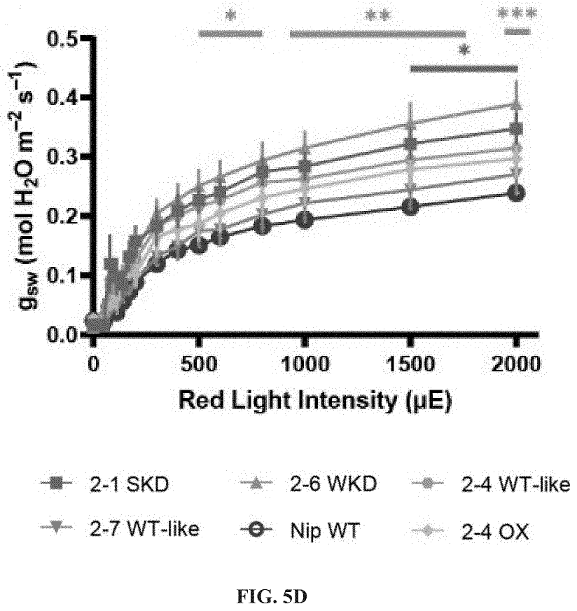

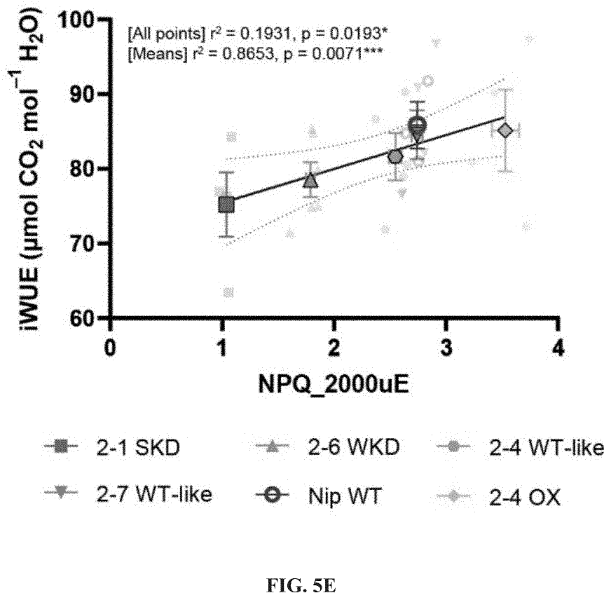

Previous work had shown that transgenic overexpression of PsbS did not compromise steady-state CO₂ assimilation in rice (Hubbart, S. et al. Enhanced thylakoid photoprotection can increase yield and canopy radiation use efficiency in rice. Commun Biol 1, 22 (2018); Hubbart, S., Ajigboye, O. O., Horton, P. & Murchie, E. H. The photoprotective protein PsbS exerts control over CO₂ assimilation rate in fluctuating light in rice: Photoprotection and CO₂ assimilation in rice. The Plant Journal (2012) doi:10.1111j.1365-313X.2012.04995.x), but it did increase intrinsic water-use efficiency (iWUE) under red-light in tobacco (Glowacka, K. et al. Photosystem II Subunit S overexpression increases the efficiency of water use in a field-grown crop. Nat Commun 9, 868 (2018)). To determine the extent to which these phenotypes are consistent in rice genotypes with varying endogenous PsbS abundance, six genotypes were assessed for steady-state chlorophyll fluorescence and gas exchange parameters under increasing red light (FIGS. 5A-5E).

Consistent with the high-throughput screening of NPQ (FIGS. 3A-3B) and acclimation to low, moderate, and high light intensities (FIGS. 4A-4J), knockdown lines 2-1_SKD and 2-6_WKD showed significantly lower NPQ at light intensities greater than 500 μmol m⁻² s⁻¹ (p<0.0001) relative to the Nipponbare WT control. In contrast, 2-4_OX showed significantly higher NPQ only at 1500 and 2000 μmol m⁻² s⁻¹ (p<0.0001) (FIG. 5A). The 2-4_WT-like line showed modest reductions in NPQ between 500 μmol m⁻² s⁻¹ and 1500 μmol m⁻² s⁻¹ (0.002<p<0.0004), though a second gene-edited WT-like control (2-7_WT-like) showed no significant differences from Nipponbare WT across all phenotypes measured (FIGS. 5A-5E). Mild knockdown in 2-6_WKD increased ΦPSII at light intensities over 500 μmol m⁻² s⁻¹ (0.041<p<0.006) (FIG. 5B).

Consistent with an increased ΦPSII, weak knockdown of PsbS also increased steady-state CO₂ assimilation at light intensities over 500 μmol m⁻² s⁻¹ with increasing statistical significance (p=0.016 ; p<0.001) (FIG. 5C). Stomatal conductance was also significantly higher in the 2-6_WKD line (p=0.043 ; p=0.0007) with a more modest increase observed in the 2-1_SKD line (p=0.043-0.048) relative to Nipponbare WT (FIG. 5D).

To assess differences in iWUE due to varying PsbS activity, high-light (>600 μmol m⁻² s⁻¹) iWUE was averaged per replicate and plotted against maximum NPQ. A significant, positive correlation (p=0.019 for all data points, p=0.007 for means) of increased iWUE with higher maximum NPQ capacity was observed (FIG. 5E).

### Q_{A} Redox State Is An Inconsistent Predictor of Rice g_{sw}

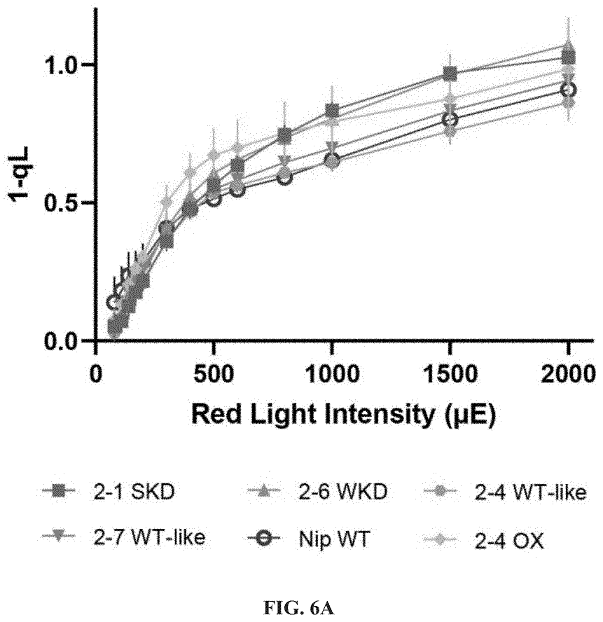

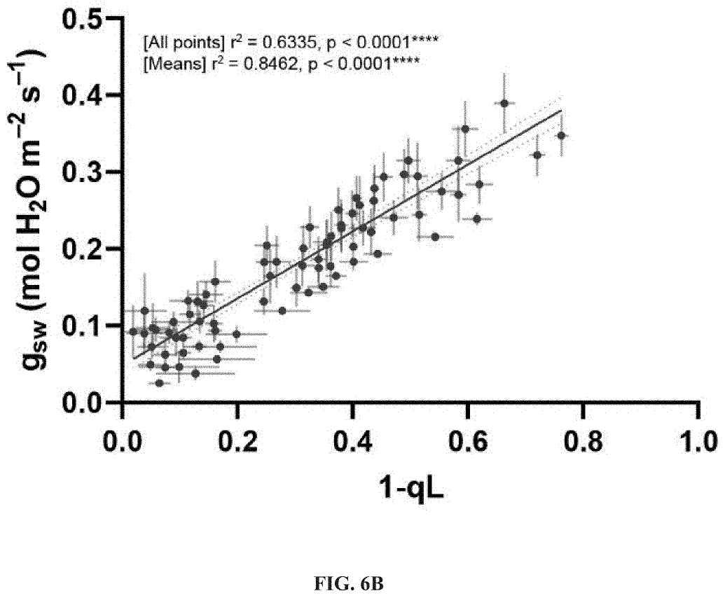

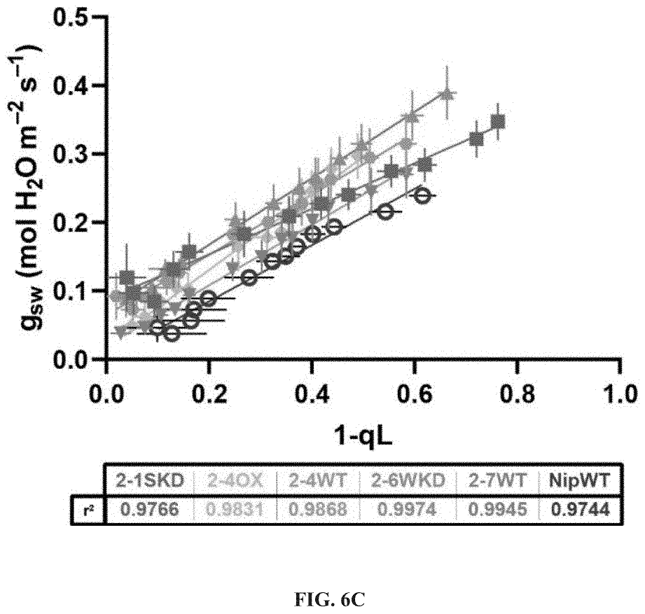

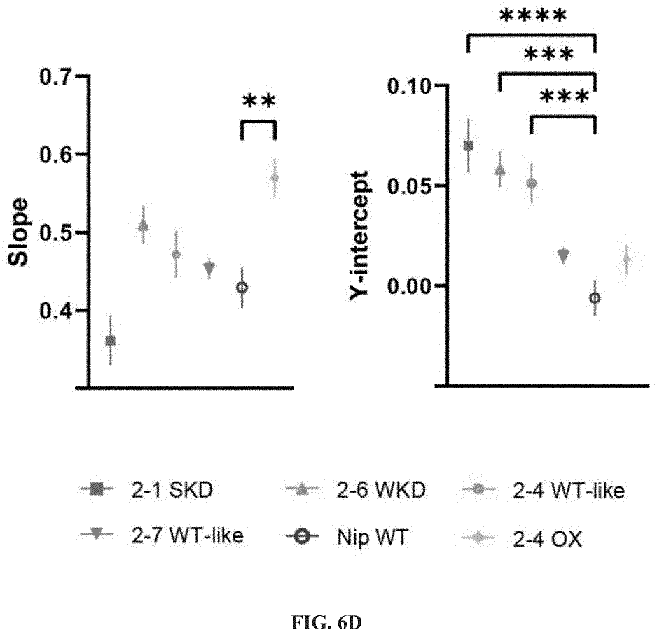

The increased iWUE phenotype observed in transgenic tobacco was predicted to be mediated by the Q_{A} redox state, approximated by 1-qL (Glowacka, K. et al. Photosystem II Subunit S overexpression increases the efficiency of water use in a field-grown crop. Nat Commun 9, 868 (2018)). Surprisingly, 1-qL did not significantly differ across lines with varying OsPsbS relative to the Nipponbare WT control (FIG. 6A).

To further interrogate the putative correlation, 1-qL was plotted against stomatal conductance for all genotypes and light intensities. While a significantly non-linear (p<0.0001) and relatively strong goodness of fit (r²=0.634 for all points, r²=0.846 for means) were observed (FIG. 6B), the correlation was weaker than what had been reported in tobacco (r²=0.98, p<0.001) (Glowacka, K. et al. Photosystem II Subunit S overexpression increases the efficiency of water use in a field-grown crop. Nat Commun 9, 868 (2018)). Leveraging the greater dynamic range in NPQ capacity uncovered in this study, the correlation was assessed individually across all six tested genotypes (FIG. 6C). While the goodness of fit significantly improved (r²>0.974), the slope of the linear regression significantly differed between Nipponbare WT and 2-4_OX (p=0.007), and the y-intercepts of the remaining non-WT lines all differed significantly (p<0.001) (FIG. 6D). In summary, all linear regressions of Q_{A} redox state and stomatal conductance, excluding that of the 2-7_WT-like control, significantly differed from Nipponbare WT as PsbS level was varied.

### Most Non-WT Phenotypes Were Explained by Variation at the 5′UTR

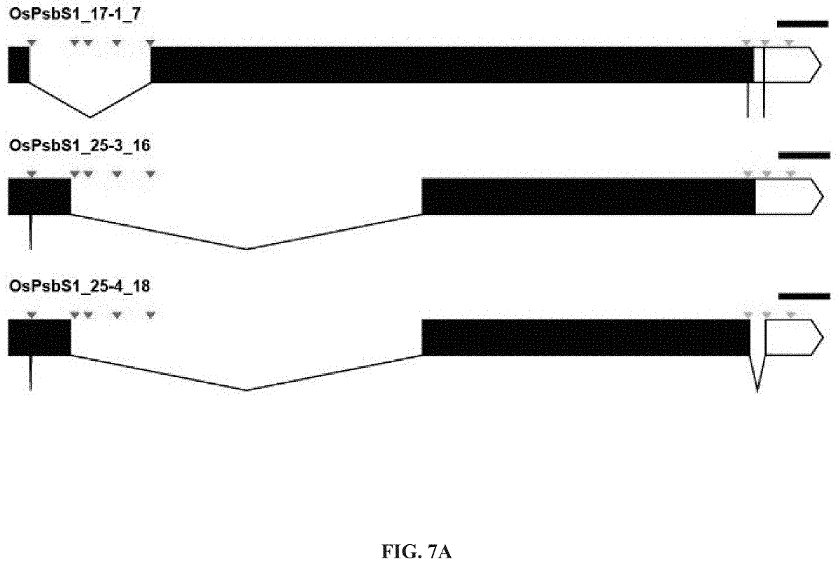

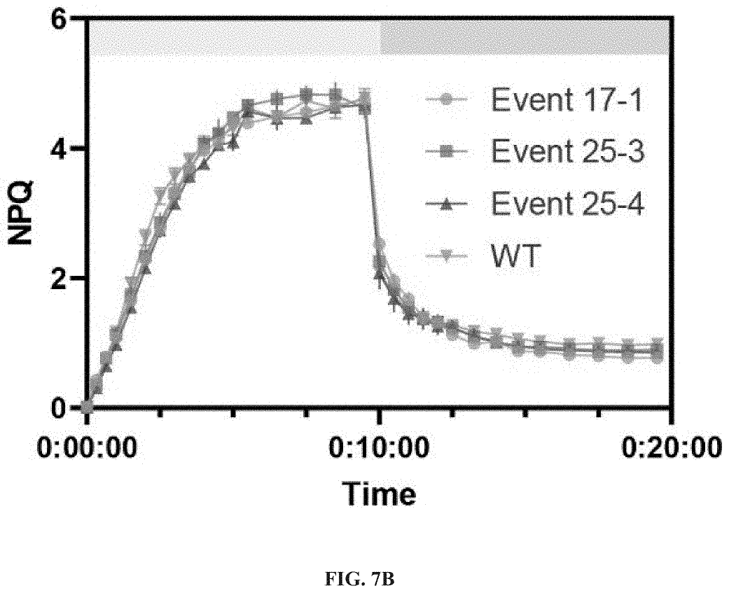

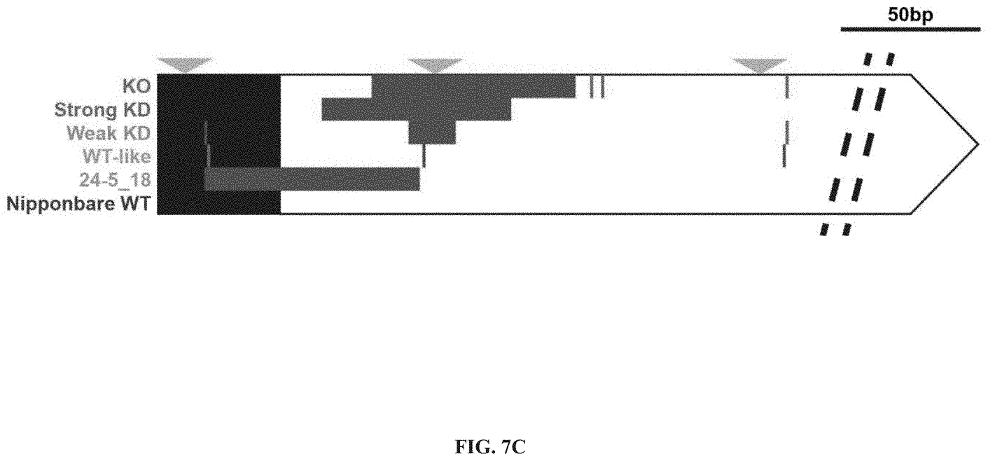

Differences in NPQ capacity, protein abundance, and iWUE substantiated observed phenotypic variation generated by non-coding sequence mutagenesis. To assess the causal mutations underlying these phenotypes, the ˜4.3-kb upstream cis-regulatory region was sequenced by Sanger sequencing. 107 of the 120 unique alleles (FIGS. 7A-7C) could be amplified by PCR (89.2%).

Three alleles containing large deletions of the five distal gRNA target sites were observed (FIG. 7A). Interestingly, all 3 lines had NPQ that was indistinguishable from WT (FIG. 7B). As a result, it is likely that most of the observed phenotypic variation can be explained by proximal gRNA variants within and near the 5′UTR. Cis-regulatory analysis of Event 2 lines with varying NPQ (FIGS. 4A-6D) revealed varying 5′UTR deletions. The size and relative location of the deletions corresponded with knockout (KO) and knockdown (KD) phenotypes, with most variation being driven by the second proximal gRNA (FIG. 7C). As shown by the 24-5-18 WT-like allele, the transcription start site (TSS) is not essential for OsPsbS1 expression.

### Long-Read Sequencing Resolves Complex Structural Variants Underlying Overexpression

Unexpectedly, neither overexpression allele (Event2-4OX or Event19-1_OX) could be analyzed by PCR genotyping. To determine whether complex, structural variants were underlying these phenotypes, a homozygous 2-4_OX T₂ line and segregating 19-1_OX T₁ line were sequenced by long-read HiFi circular consensus sequencing (Pacific Biosciences). The 19-1 allele was sequenced in the T₁ generation due to unexpected sterility, also found in 5 of the originally 28 independent transformants (FIG. 1B). Reads were assembled de novo and mapped onto the Nipponbare O. sativa v7.0 reference genome (Hellemans, J., Mortier, G., De Paepe, A., Speleman, F. & Vandesompele, J. qBase relative quantification framework and software for management and automated analysis of real-time quantitative PCR data. Genome Biol 8, R19 (2007)) using bioinformatic tools developed by Pacific Biosciences (See Materials and Methods, Error! Reference source not found.).

First, all sequence and structural variants across the 12 rice chromosomes were visualized by dot plots. In particular, dot plots of 2-4_OX and 19-1_OX plotting sequenced OX variants (x-axis) against the reference genome (y-axis) were generated (data not shown). The OX sequences were found to be largely identical to the reference genome. Small chromosomal variants (e.g., indels, SNPs) were found to have a highly consistent pattern between 2-4_OX and 19-1_OX, which likely represented inherent sequence differences between the reference genome and lab strain used. The two sequenced OX strains did show modest differences in chromosome-level structure, indicated by gaps in continuity that indicate larger insertions and deletions.

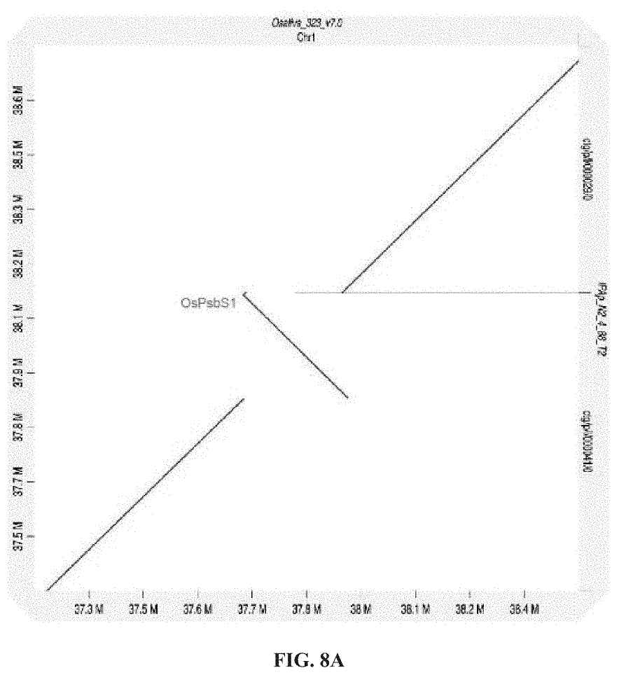

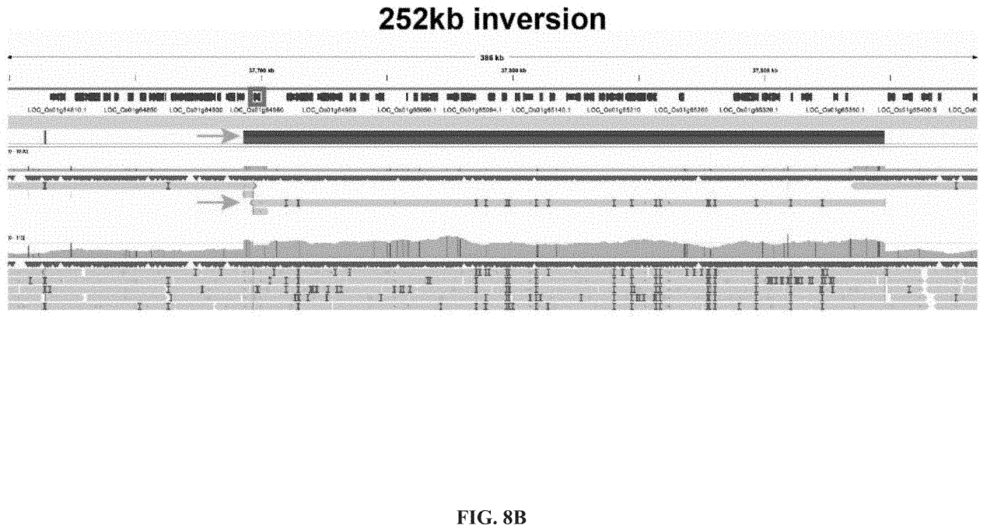

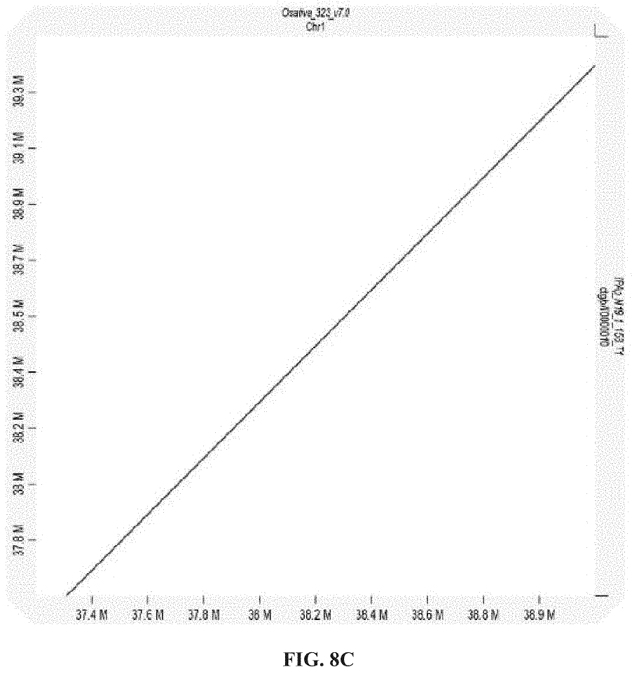

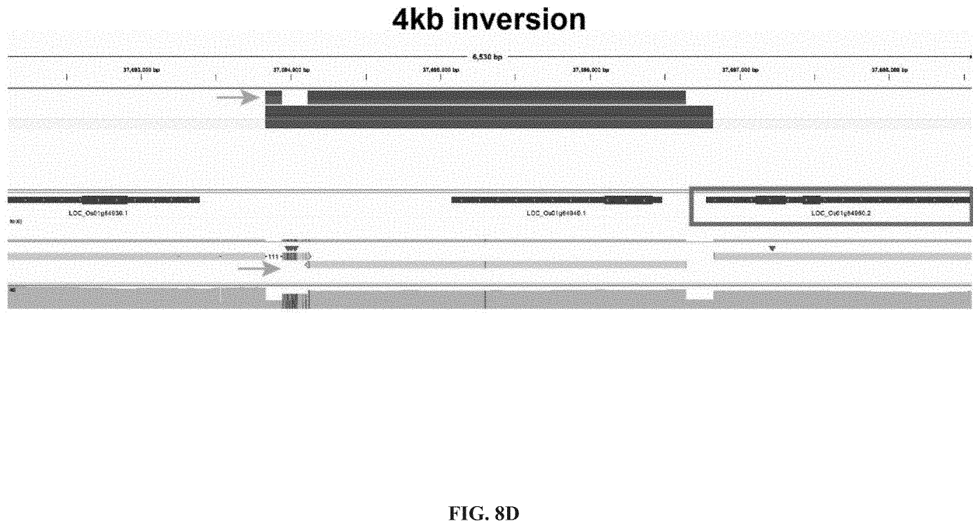

Increased resolution of the OsPsbS1 Chromosome 1 locus highlighted large differences in structural variation not observable at the whole-genome scale. In FIG. 8A, an increased resolution dot plot of the Chr. 1 locus (1.1 Mbp) harboring OsPsbS1 for the 2-4_OX line is shown, with the break in continuity signifying the presence of a genomic inversion. This was further substantiated at the sequence level by visualization using the Integrative Genome Viewer (IGV). FIG. 8B shows increased resolution of the Chr. 1 locus with the ˜254 kb inversion (Chr1:37693233-37948089) upstream of OsPsbS1 for the 2-4_OX line. In contrast, the 19-1_OX line showed no appreciable differences in an increased resolution dot plot of the Chr. 1 locus (˜1.5 Mbp) harboring OsPsbS1 (FIG. 8C). However, increased resolution of the Chr. 1 locus for the 19-1_OX line revealed the presence of a ˜3-4 kb inversion (Chr1:˜37693800-37696800) upstream of OsPsbS1 (FIG. 8D). The exact junction points of the inversion were unresolved by long-read sequencing, but corresponded to the region between gRNA5 and gRNA7 listed in Table 1. Thus, inversions of varying sizes accounted for observed PsbS overexpression phenotypes.

### Overexpression Phenotype Is Constitutive, But Expression Is Not

In FIGS. 9A-9B, the expression levels of OsPsbS1 in a sample taken at one time point are shown. In FIG. 9A, it can be seen that the 2-4_OX line had significantly higher expression of OsPsbS1 than WT (p<0.01), and the 2-5 _KO line had significantly lower OsPsbS1 transcript levels and was below the threshold of detection. In FIG. 9B, however, the expression levels in the 2-4_OX, WT, and 2-4_WT-like lines were comparable to each other, while the expression levels in the 2-6_WKD, 2-1_SKD, and 2-5_KO lines were lower than WT, at a statistically significant level (0.01 <p<0.0001). In order to elucidate the differences observed, a growth chamber time course was done with 2-4_OX and WT plants. The results of this time course, shown in FIG. 9C, revealed that expression in the OX line peaked in the morning and declined over the course of the day, with statistically significant differences in expression only observed in the first timepoint (1 hr, p<0.01). In contrast, diurnal NPQ measurements showed that NPQ levels remained high in the 2-4_OX line over the course of the day as compared to WT, independent of variation seen in OsPsbS1 transcript level. These results unexpectedly indicated that the overexpression phenotype (i.e., NPQ) was constitutive, but the overexpression of OsPsbS1 was not.

### Discussion

As the understanding of basic biology grows, so does the need for specific and efficient gene-editing tools to modify biology for application in agriculture and medicine. The CRISPR/Cas9 toolkit has the potential to meet that need if the design principles to modulate gene expression are well understood. The results shown in this example demonstrate the ability to use CRISPR/Cas9 mutagenesis of non-coding sequences to achieve native overexpression of genes at levels that compete with transgenic overexpression. The results obtained were facilitated by a high-throughput screening pipeline for rapid detection of homozygous, Cas9-free progeny, which revealed a diverse array of quantitative phenotypes.

Consistent with a large body of literature, it was found that overexpression of PsbS increases NPQ capacity (Li, X.-P., Müller-Moulé, P., Gilmore, A. M. & Niyogi, K. K. PsbS-dependent enhancement of feedback de-excitation protects photosystem II from photoinhibition. Proceedings of the National Academy of Sciences 99, 15222-15227 (2002); Hubbart, S. et al. Enhanced thylakoid photoprotection can increase yield and canopy radiation use efficiency in rice. Commun Biol 1, 22 (2018); Glowacka, K. et al. Photosystem II Subunit S overexpression increases the efficiency of water use in a field-grown crop. Nat Commun 9, 868 (2018)).

While the phenotypes observed reinforce observed differences in agronomic traits dependent on PsbS activity, the most important gains from these results came from the structural variants underlying changes in gene expression. Due to the distribution of gRNA sites in the multiplex experimental design (FIG. 1A), it was found that distal regions of the OsPsbS1 promoter, which are conserved between indica and japonica subspecies, were dispensable for expression of OsPsbS1 (FIG. 7B), and much of the phenotypic variation observed was due to indels near and within the 5′UTR.

Surprisingly, the two overexpression alleles that increased NPQ capacity were driven by inversions upstream of OsPsbS1 (FIGS. 8A-8F). Allele 19-1_OX presented an interesting example as it carried a clean inversion between the distal and proximal gRNAs that may readily be replicated or introgressed into other varieties.

A revolution in long-read sequencing has revealed the pervasiveness of genomic structural variants. Complex structural variants (CSVs) including translocations, insertions, and inversions are persistent at both the population and pan-genome level as reported in tomato (Alonge, M. et al. Major Impacts of Widespread Structural Variation on Gene Expression and Crop Improvement in Tomato. Cell 182, 145-161.e23 (2020)), rapeseed (Song, J.-M. et al. Eight high-quality genomes reveal pan-genome architecture and ecotype differentiation of Brassica napus. Nat. Plants 6, 34-45 (2020)), maize (Sun, S. et al. Extensive intraspecific gene order and gene structural variations between Mo17 and other maize genomes. Nat Genet 50, 1289-1295 (2018)), grapevine (Zhou, Y. et al. The population genetics of structural variants in grapevine domestication. Nat. Plants 5, 965-979 (2019)), and rice (Qin, P. et al. Pan-genome analysis of 33 genetically diverse rice accessions reveals hidden genomic variations. Cell 184, 3542-3558.e16 (2021)), affecting upwards of 20% of all genes. In fact, much of the current understanding of structural variants is driven by the prevalence of these changes driving various human cancers and diseases (Weischenfeldt, J., Symmons, O., Spitz, F. & Korbel, J. O. Phenotypic impact of genomic structural variation: insights from and for human disease. Nat Rev Genet 14, 125-138 (2013); Beyter, D. et al. Long-read sequencing of 3,622 Icelanders provides insight into the role of structural variants in human diseases and other traits. Nat Genet 53, 779-786 (2021); Hehir-Kwa, J. Y. et al. A high-quality human reference panel reveals the complexity and distribution of genomic structural variants. Nat Commun 7, 12989 (2016)). Unlike humans however, plants exhibit a much greater tolerance to (and thus abundance of) structural variants that is likely driven in vivo by transposable elements and recombination—both documented as key drivers of crop domestication (Dominguez, M. et al. The impact of transposable elements on tomato diversity. Nat Commun 11, 4058 (2020); Gaut, B. S., Seymour, D. K., Liu, Q. & Zhou, Y. Demography and its effects on genomic variation in crop domestication. Nature Plants 4, 512-520 (2018)).

In fact, chromosomal inversions have also been implicated in gene overexpression during the domestication of peach (Zhou, H. et al. A 1.7-Mb chromosomal inversion downstream of a PpOFP1 gene is responsible for flat fruit shape in peach. Plant Biotechnology Journal 19, 192-205 (2021)) and the complex rearrangements that underlie multiple myeloma (Affer, M. et al. Promiscuous MYC locus rearrangements hijack enhancers but mostly super-enhancers to dysregulate MYC expression in multiple myeloma. Leukemia 28, 1725-1735 (2014)). Recently, Lu et. al. showed that CRISPR/Cas9 could be used to drive native overexpression via promoter swapping (Lu, Y. et al. A donor-DNA-free CRISPR/Cas-based approach to gene knock-up in rice. Nat. Plants 7, 1445-1452 (2021)), generating inversions in ˜3% of transformed calli that increased gene expression of OsPPO1 at varying frequencies. However, these inversions came at the cost of expression of the opposite promoter, knocking out gene expression of the target Calvin-Benson cycle protein 12 (OsCP12) gene (LOC_Os01g19740). In the instance of 19-1_OX allele, the other inversion breakpoint occurred downstream of the 3′UTR of the neighboring gene (LOC_Os01g64930) and likely did not interfere with its expression. The work presented here, unbiased in its target design, reinforces and expands the genome-engineering potential of inversions for native gene overexpression.

In the dataset, overexpression alleles harboring an inversion accounted for 2 of 13 unique alleles that could not be PCR amplified and sequenced. Of the remaining 11 alleles, 8 were phenotypic knockouts, 1 was a phenotypic knockdown, and 2 were WT-like in NPQ activity. It is possible that CSVs also underlie these remaining alleles, increasing the potential frequency of CSV alleles from 1.7% to 10.8% by CRISPR/Cas9.

### Example 2: Promoter and 5′UTR Mutagenesis of OsVDE/OsZEP in the Nipponbare 2-4 OX PsbS Line

This example describes targeting O. sativa VDE (OsVDE, LOC_Os04g31040) and ZEP (OsZEP, LOC_Os04g37619) via CRISPR/Cas9 mutagenesis in order to recapitulate the VPZ phenotype via altered endogenous gene expression (Kromdijk J, Glowacka K, Leonelli L, et al. Improving photosynthesis and crop productivity by accelerating recovery from photoprotection. Science. 2016;354(6314):857-862). The PsbS OX line described in Example 1 is used as the recipient parent, and OsVDE and OsZEP are individually targeted in separate pools of transformants to identify overexpression alleles in each of those traits. Candidate OX and knockdown (KD) alleles are crossed and assessed for faster relaxation of NPQ.

### Materials and Methods

### Guide RNA Design and Construct Cloning

Five to eight guide RNA (gRNA) target sites are identified within the promoter and 5′UTR upstream of OsVDE and OsZEP. The promoter is broadly defined as the 2 kb region upstream of the start codon (ATG) of the gene of interest, though in the case of OsVDE, an upstream gene constrains the putative promoter and 5′UTR to ˜750 bp. Candidate gRNAs are identified upstream of the gene of interest using CRISPR-P (crispr[dot]hzau[dot]edu[dot]cn) and selected for 1) high specificity to reduce risk of off-targets and 2) even distribution across the target site(s) of interest. For target sites significantly larger than 300 bp, gRNAs are identified every ˜200-300 bp. For target sites <300 bp, gRNAs are spaced every ˜50-150 bp to ensure at least two gRNAs are targeted to loci of interest (e.g. 5′UTR). Table 4 and Table 5 provide the gRNA targets for OsVDE and OsZEP, respectively.

The eight gRNAs are assembled into a DNA cassette interspersed with scaffolds and tRNA linkers for polycistronic gRNA expression as previously described (Xie K, Minkenberg B, Yang Y. Boosting CRISPR/Cas9 multiplex editing capability with the endogenous tRNA-processing system. Proc Natl Acad Sci. 2015; 112(11):3570-3575. doi:10.1073/pnas.1420294112) for synthesis (Genscript). The insert is cloned into the pRGEB32 rice Agrobacterium-mediated transformation vector (Addgene Plasmid #63142) via GoldenGate Assembly for CRISPR/Cas9 editing by Agrobacterium-mediated transformation of rice.

TABLE 4
gRNA targets for promoter and 5′UTR of OsVDE (LOC_Os04g31040)

| | | | Position from ATG | |
|---|---|---|---|---|
| | Orientation | | in O. sativa ssp. | |
| | (relative to | Target | japonica cultivar | Spacer Sequence |
| Insert | ORF) | site | Nipponbare | (5′ -> 3′) |
| gRNA1 | F | Promoter | -705:-685 | GCCACGTCGGGCCAAACGGG |
| | | | | (SEQ ID NO: 25) |
| gRNA2 | F | Promoter | -408:-388 | ACAACATATGGAAAAATCGG |
| | | | | (SEQ ID NO: 26) |
| gRNA3 | R | Promoter | -188:-208 | AATGGGCCGGGCCAAGGCCT |
| | | | | (SEQ ID NO: 27) |
| gRNA4 | F | 5'UTR | -125:-105 | AGCCAAGCCAAGCCCCTCCG |
| | | | | (SEQ ID NO: 28) |
| gRNA5 | R | 5'UTR | -39:-59 | GGGATCGAGAGCTCGAGCAG |
| | | | | (SEQ ID NO: 29) |

| | |
|---|---|
| gRNA | GTTTTAGAGCTAGAAATAGCAAGTTAAAATAAGGCTAGTCCGTTATCAACTTGAA |
| scaffold | AAAGTGGCACCGAGTCGGTGC (SEQ ID NO: 9) |
| tRNA | AACAAAGCACCAGTGGTCTAGTGGTAGAATAGTACCCTGCCACGGTACAGACCCG |
| linker | GGTTCGATTCCCGGCTGGTGCA (SEQ ID NO: 10) |

TABLE 5
gRNA targets for promoter and 5′UTR of OsZEP (LOC_Os04g37619)

| | | | Position from ATG | |
|---|---|---|---|---|
| | Orientation | | in O. sativa ssp. | |
| | (rel. to | Target | japonica cultivar | Spacer Sequence |
| Insert | ORF) | site | Nipponbare | (5′ -> 3′) |
| gRNA1 | F | Promoter | -1806:-1786 | CCAAACCTTTACAAACCGCT |
| | | | | (SEQ ID NO: 30) |
| gRNA2 | F | Promoter | -1561:-1541 | TTAACCTTATAGGTTGAAAT |
| | | | | (SEQ ID NO: 31) |
| gRNA3 | F | Promoter | -1197:-1177 | GGGAGTATATACCTTTAGGG |
| | | | | (SEQ ID NO: 32) |
| gRNA4 | R | Promoter | -965:-985 | TTCTCACCGTTAATTCTAAA |
| | | | | (SEQ ID NO: 33) |
| gRNA5 | R | Promoter | -705:-725 | TTGTGGAACACAGTTCATGG |
| | | | | (SEQ ID NO: 34) |
| gRNA6 | F | Promoter | -384:-364 | ACACATGGTTAGCCACCCAG |
| | | | | (SEQ ID NO: 35) |
| gRNA7 | R | 5′UTR | -180:-200 | TTTGGGCTGCGGTCGTGGAA |
| | | | | (SEQ ID NO: 36) |
| gRNA8 | R | 5′UTR | -88:-108 | GTGTAGCGGAGACGGAGCA |
| | | | | A (SEQ ID NO: 37) |

| | |
|---|---|
| gRNA | GTTTTAGAGCTAGAAATAGCAAGTTAAAATAAGGCTAGTCCGTTATCAACTTGAA |
| scaffold | AAAGTGGCACCGAGTCGGTGC (SEQ ID NO: 9) |
| tRNA | AACAAAGCACCAGTGGTCTAGTGGTAGAATAGTACCCTGCCACGGTACAGACCCG |
| linker | GGTTCGATTCCCGGCTGGTGCA (SEQ ID NO: 10) |

### Induction and Transformation of Embryogenic Calli

Embryogenic calli of O. sativa ssp. japonicaNipponbare 2-4 (the PsbS OX allele described in Example 1) are prepared and transformed as described in Example 1.

Once transformed, T₀ plants are regenerated from the calli. These T₀ plants are then selfed to produce T₁ progeny, which are screened for differences in NPQ phenotype and gene expression as described in Example 1 to identify stable homozygous lines with heritable edited alleles.

### Example 3: Non-Coding Sequence Mutagenesis of Arabidopsis thaliana PsbS and VDE to Identify Efficient Target Sites for Overexpression

The editing approach in Nipponbare (O. sativa ssp. japonica) split the 8 original gRNAs across two sites either distal (3-4 kb) or proximal (150-400 bp) to the OsPsbS1 start codon. Interestingly, the results described in Example 1 found that all 5 distal gRNA sites are dispensable—a deletion spanning all 5 sites results in progeny with WT NPQ (e.g., Event 25-4). Accordingly, it is hypothesized that the 3 gRNAs within and immediately upstream of the 5′UTR are driving the observed phenotypic variation, including the significant overexpression of OsPsbS1 transcript and protein in O. sativa ssp. japonica Nipponbare Event 2-4 (PsbS OX allele).

To determine whether non-coding sequence (NCS, i.e., 5′UTR, 3′UTR, intron) mutagenesis has greater promise in generating higher frequencies and/or magnitudes of overexpression, the NCS of the A. thaliana PsbS (AtPsbS, At1g44575) and VDE (AtVDE, At1g08550) genes, whose downstream phenotypes can be rapidly resolved by chlorophyll fluorescence imaging, is edited. By quantifying the relative activity of the mutant alleles produced in stably edited lines, future design strategies in crops are informed.

Interestingly, AtVDE is in a gene dense region and has no unique promoter that can be mutagenized without disrupting adjacent genes. Similarly, the 5′UTR of AtPsbS is short (73 bp), with few possible gRNA targets for mutagenesis. This multi-targeted approach will therefore identify multiple avenues for overexpression that may also expand the diversity of target sites in other species and genes when genomic structure is limiting.

### Materials and Methods

### Guide RNA Design and Construct Cloning

Guide RNA (gRNA) target sites are identified within NCS of AtPsbS and AtVDE using CRISPR-P (crispr[dot]hzau[dot]edu[dot]cn) and selected for 1) high specificity to reduce off-targets and 2) even distribution across the target site(s) of interest. For target sites significantly larger than 300 bp, gRNAs are identified every ˜200-300 bp. For target sites <300 bp, gRNAs are spaced every ˜50-150 bp to ensure at least two gRNAs are targeted to loci of interest (e.g. 5′UTR). Table 6 provides an overview of the fragment length and gRNA targets for NCS mutagenesis ofAtPsbS and AtVDE. Table 7 and Table 8 provide the gRNA targets for AtPsbS and AtVDE, respectively.

The eight gRNAs are assembled into a DNA cassette interspersed with scaffolds and tRNA linkers for polycistronic gRNA expression as previously described (Xie K, Minkenberg B, Yang Y. Boosting CRISPR/Cas9 multiplex editing capability with the endogenous tRNA-processing system. Proc Natl Acad Sci. 2015; 112(11):3570-3575. doi:10.1073/pnas.1420294112) for synthesis (Genscript). The insert is cloned into the pKI1.1R Arabidopsis Agrobacterium-mediated transformation vector (Addgene Plasmid #85808) via GoldenGate Assembly for CRISPR/Cas9 editing by Agrobacterium-mediated transformation.

TABLE 6
Fragment length and associated number of gRNA
targets for AtPsbS/AtVDE NCS mutagenesis

| | Promoter | 5′ UTR | Introns | 3′ UTR |
|---|---|---|---|---|
| AtPsbS | 8 gRNA - | 2 gRNA - | 4 gRNA - | 3 gRNA - |
| | 2 kb | 73 bp | 629 bp | 189 bp |
| AtVDE | No possible | 4 gRNA - | 5 gRNA - | 2 gRNA - |
| | targets | 243 bp | 832 bp | 144 bp |

TABLE 7
gRNA targets for NCS mutagenesis of AtPsbS (At1g44575) with a promoter-
targeted control

| | Orientation | | | | |
|---|---|---|---|---|---|
| | (rel. to | Target | Element | Position from ATG | Spacer Sequence |
| Insert | ORF) | site | length | in A. thaliana Col-0 | (5′ -> 3′) |
| gRNA1a | F | Promoter | 2031 bp | -2031:-2011 | AGTTGCCCAAAAAAAGAAGA |
| | | | | | (SEQ ID NO: 38) |
| gRNA2a | F | Promoter | | -1769:-1749 | GCTTGTCATGATGTGAGATG |
| | | | | | (SEQ ID NO: 39) |
| gRNA3a | R | Promoter | | -1435:-1455 | TTATTTAATTACTATCGCAC |
| | | | | | (SEQ ID NO: 40) |
| gRNA4a | R | Promoter | | -1211:-1231 | GTAATTCTTTGACTATTTCA |
| | | | | | (SEQ ID NO: 41) |
| gRNA5a | R | Promoter | | -838:-858 | GCAGCGTTTGCGGTTGGTAG |
| | | | | | (SEQ ID NO: 42) |
| gRNA6a | R | Promoter | | -634:-654 | GAGCCGGGCACAAACACAAG |
| | | | | | (SEQ ID NO: 43) |
| gRNA7a | R | Promoter | | -423:-443 | ATAGAAGATTCACGTCAAAA |
| | | | | | (SEQ ID NO: 44) |
| gRNA8a | F | Promoter | | -138:-118 | GAGAGTATGGAAAGGACAAT |
| | | | | | (SEQ ID NO: 45) |
| gRNA1b | R | 5′UTR | 73 bp | -55:-75 | TGAAGAGATTAAAAGATATA |
| | | | | | (SEQ ID NO: 46) |
| gRNA2b | R | 5′UTR | | +1:-19 | TTCTTTCTGAGGATGAGAGA |
| | | | | | (SEQ ID NO: 47) |
| gRNA1c | F | Intron_1 | 629 bp | +201:+221 | TACTCTTTACTTGTCCCACA |
| | | | | | (SEQ ID NO: 48) |
| gRNA2c | R | Intron_2 | | +419:+399 | AGTTAGTGTATTTCATAGAG |
| | | | | | (SEQ ID NO: 49) |
| gRNA3c | F | Intron_3 | | +825:+845 | CCTAAGTGTAGTGTCCGGAT |
| | | | | | (SEQ ID NO: 50) |
| gRNA4c | F | Intron_3 | | +995:+1015 | TTTCCCCCATGTAAGCACAT |
| | | | | | (SEQ ID NO: 51) |
| gRNA1d | F | 3′UTR | 189 bp | +1466:+1486 | TCTCTTCATGTTGAGACAAA |
| | | | | | (SEQ ID NO: 52) |
| gRNA2d | R | 3′UTR | | +1546:+1526 | AATTCCAAGTTAAAAACAAA |
| | | | | | (SEQ ID NO: 53) |
| gRNA3d | R | 3′UTR | | +1595:+1575 | TATCAATCACCTAGTCACTT |
| | | | | | (SEQ ID NO: 54) |

| | |
|---|---|
| gRNA | GTTTTAGAGCTAGAAATAGCAAGTTAAAATAAGGCTAGTCCGTTATCAACTTGAAAAAGTG |
| scaffold | GCACCGAGTCGGTGC (SEQ ID NO: 9) |
| tRNA | AACAAAGCACCAGTGGTCTAGTGGTAGAATAGTACCCTGCCACGGTACAGACCCGGGTTC |
| linker | GATTCCCGGCTGGTGCA (SEQ ID NO: 10) |

TABLE 8
gRNA targets for NCS mutagenesis of AtVDE (At1g08550)

| | Orientation | | | Position from | |
|---|---|---|---|---|---|
| | (rel. to | Target | Element | ATG in A. | Spacer Sequence |
| Insert | ORF) | site | length | thaliana Col-0 | (5′ -> 3′) |
| gRNA1b | F | 5′UTR_1 | 243 bp | -528:-508 | GGTGGAGAAAACAACCGCCT |
| | | | | | (SEQ ID NO: 55) |
| gRNA2b | F | 5′UTR_1 | | -468:-448 | TTTGTTCACACACCACACGA |
| | | | | | (SEQ ID NO: 56) |
| gRNA3b | R | 5′UTR_1 | | -335:-355 | AACAAAAACTGAAAGCTCCG |
| | | | | | (SEQ ID NO: 57) |
| gRNA4b | F | 5′UTR_2 | | -44:-24 | ACTCAGGTATTGCTTGGTGT |
| | | | | | (SEQ ID NO: 58) |
| gRNA1c | R | Intron_1 | 832 bp | -157:-177 | AACCAGGAATCATAAAACGT |
| | | | | | (SEQ ID NO: 59) |
| gRNA2c | F | Intron_3 | | +491:+511 | TAGTGTCCCCCCACCAAAAC |
| | | | | | (SEQ ID NO: 60) |
| gRNA3c | R | Intron_3 | | +689:+669 | GAGGGAAATATATAAATTGA |
| | | | | | (SEQ ID NO: 61) |
| gRNA4c | R | Intron_4 | | +908:+888 | ATAATTATAAACAAAATCAT |
| | | | | | (SEQ ID NO: 62) |
| gRNA5c | F | Intron_5 | | +1327:+1347 | ACATCTTGTATCCACCCACG |
| | | | | | (SEQ ID NO: 63) |
| gRNA1d | R | 3′ UTR | 144 bp | +1958:+1938 | TATAGTTTGTACAACAATGG |
| | | | | | (SEQ ID NO: 64) |
| gRNA2d | F | 3′ UTR | | +2024:+2044 | AATTGGATACAGAAAACACA |
| | | | | | (SEQ ID NO: 65) |

| | |
|---|---|
| gRNA | GTTTTAGAGCTAGAAATAGCAAGTTAAAATAAGGCTAGTCCGTTATCAACTTGAAAAAGTG |
| scaffold | GCACCGAGTCGGTGC (SEQ ID NO: 9) |
| tRNA | AACAAAGCACCAGTGGTCTAGTGGTAGAATAGTACCCTGCCACGGTACAGACCCGGGTTCG |
| linker | ATTCCCGGCTGGTGCA (SEQ ID NO: 10) |

### Transformation and Phenotyping of A. thaliana

WT A. thaliana Col-0 is transformed with each of the 7 constructs summarized in Table 6. T₁ seed carrying Cas9 and gRNAs for each of the target sites (i.e. promoter, 5′UTR, 3′UTR or introns) is collected and transformants are identified.

After the transformant lines are selfed, phenotyping of stable, heritable, homozygous edited alleles across the regions of interest is performed as described in Example 1. The phenotyping identifies differences in the abundance and magnitude of edited overexpression alleles across different NCS target sites.

### Example 4: Non-Coding Sequence Mutagenesis of Oryza sativa, Zea mays, and Vigna unguiculata PsbS

This example describes non-coding sequence (NCS) mutagenesis in Oryza sativa, Zea mays, and Vigna unguiculata PsbS (i.e., OsPsbS, ZmPsbS, and VuPsbS).

### Materials and Methods

### Guide RNA Design and Construct Cloning

Guide RNA (gRNA) target sites are identified within NCS of OsPsbS, ZmPsbS, and VuPsbS as described in Example 3. The numbering of gRNA reflects CRISPR-P output to track top candidate off-target loci. Table 9, Table 10, and Table 11 provide the gRNA targets for OsPsbS, ZmPsbS, and VuPsbS, respectively. For OsPsbS, the 5′UTR gRNA are identical to the two used in the initial construct to generate PsbS OX alleles described in Example 1.

Construct cloning is done as described in Example 3.

TABLE 9
gRNA targets for NCS mutagenesis of OsPsbS1 (Os01g64960)

| | Orientation | | | Position from | |
|---|---|---|---|---|---|
| | (relative | Target | Element | ATG in O. sativa | Spacer Sequence |
| Insert | to ORF) | site | length | (Nipponbare) | (5′ -> 3′) |
| gRNA7 | R | 5′UTR | 331 bp | -263:-283 | GTCTCCCCGAATCCCTTCTA |
| (current | | | | | (SEQ ID NO: 7) |
| design) | | | | | |
| gRNA8 | F | 5′UTR | | -166:-146 | CTACGCCTCCCACCCGCCAC |
| (current | | | | | (SEQ ID NO: 8) |
| design) | | | | | |
| gRNA57 | F | Intron_1 | 1545 bp | +191:+221 | AAGAAAGGTTGGAATTTGGA |
| | | | | | (SEQ ID NO: 66) |
| gRNA27 | F | Intron_2 | | +565:+585 | TTAACGGAAGCACTTTACCC |
| | | | | | (SEQ ID NO: 67) |
| gRNA22 | R | Intron_2 | | +1057:+1037 | TCTTTGTTATTGCTGCAGGA |
| | | | | | (SEQ ID NO: 68) |
| gRNA5 | F | Intron_2 | | +1228:+1248 | AGTGCGAGTAGCATTCTAGT |
| | | | | | (SEQ ID NO: 69) |
| gRNA72 | R | Intron_2 | | +1545:+1525 | TATATAGTGTGGCAAGATGG |
| | | | | | (SEQ ID NO: 70) |
| gRNA107 | R | Intron_2 | | +1801:+1781 | TGAGCGCTTTTGCATCAGGT |
| | | | | | (SEQ ID NO: 71) |
| gRNA45 | R | 3′UTR | 342 bp | +2359:+2339 | ATGCGCAAGCACAACAATGG |
| | | | | | (SEQ ID NO: 72) |
| gRNA23 | F | 3′UTR | | +2463:+2483 | TGAGATTTAGCTACTTGCTG |
| | | | | | (SEQ ID NO: 73) |

TABLE 10
gRNA targets for NCS mutagenesis of ZmPsbS (Zm00001d042697)

| | Orientation | | | Position from | |
|---|---|---|---|---|---|
| | (relative | | Element | ATG in Z. mays | Spacer Sequence |
| Insert | to ORF) | Target site | length | (AGPv4) | (5′ -> 3′) |
| gRNA14 | R | 5′UTR | 188 bp | -124:-144 | GGTAGTCCTAGGCGAGCGCG |
| | | | | | (SEQ ID NO: 74) |
| gRNA6 | R | 5′UTR | | -42:-62 | GCACAGAGACGGATAAAGAG |
| | | | | | (SEQ ID NO: 75) |
| gRNA3 | R | Intron_1 | 1234 bp | +299:+279 | GCATGCGTGCACATAACACA |
| | | | | | (SEQ ID NO: 76) |
| gRNA33 | R | Intron_2 | | +591:+571 | CAACGACTTATAATTTCGGA |
| | | | | | (SEQ ID NO: 77) |
| gRNA98 | F | Intron_2 | | +781:+801 | TTTATAATTTAGACTTGGAG |
| | | | | | (SEQ ID NO: 78) |
| gRNA4 | F | Intron_2 | | +1186:+1206 | GGGTGGAAGAAAATTCCAAA |
| | | | | | (SEQ ID NO: 79) |
| gRNA78 | F | Intron_3 | | +1769:+1789 | TATAATAATGATGGTGCCTG |
| | | | | | (SEQ ID NO: 80) |
| gRNA74 | F | 3′UTR | 214 bp | +2164:+2184 | ACGCACACGACTCATGTCCG |
| | | | | | (SEQ ID NO: 81) |
| gRNA70 | F | 3′UTR | | +2235:+2255 | TACTACTAGCTAATATGCCA |
| | | | | | (SEQ ID NO: 82) |

TABLE 11
gRNA targets for NCS mutagenesis of VuPsbS (Vigun09g165900)

| | | | | Position from | |
|---|---|---|---|---|---|
| | Orientation | | | ATG in | |
| | (relative | | Element | V. unguiculata | Spacer Sequence |
| Insert | to ORF) | Target site | length | v1.1 | (5′ -> 3′) |
| gRNA47 | F | 5′UTR | 339 bp | -287:-267 | GAGTGTACTAAGTTTGAG |
| | | | | | GC (SEQ ID NO: 83) |
| gRNA8 | R | 5′UTR | | -115:-135 | CACCATCTATAATTTAACA |
| | | | | | A (SEQ ID NO: 84) |
| gRNA2 | F | 5′UTR | | -53:-33 | CATTCTGAACCAAACCAC |
| | | | | | CG (SEQ ID NO: 85) |
| gRNA52 | R | Intron_1 | 272 bp | +244:+224 | GTTTCATTTACATAAAAGA |
| | | | | | A (SEQ ID NO: 86) |
| gRNA15 | F | Intron_2 | | +426:+446 | CCTTGTAATTTAATAAACA |
| | | | | | G (SEQ ID NO: 87) |
| gRNA42 | F | Intron_3 | | +800:+820 | ATAGAAGAGTAACATTTG |
| | | | | | TT (SEQ ID NO: 88) |
| gRNA16 | R | 3′UTR | 263 bp | +1149:+1129 | GCATTGATGATAGATAAG |
| | | | | | GA (SEQ ID NO: 89) |
| gRNA6 | R | 3′UTR | | +1300:+1280 | GTTGCGCCGACACCAAGC |
| | | | | | AA (SEQ ID NO: 90) |

### Discussion

In the simplest case, overexpression by CRISPR/Cas9 will be achieved through the mutation or deletion of a repressive cis-regulatory element. However, the existence and maintenance of such a repressor would likely be dictated by natural selection. It is hypothesized that CRISPR/Cas9 can also be used to drive evolution at the gene(s) of interest (e.g. in the non-coding sequences of PsbS) in order to generate novel genomic variation that generates overexpression phenotypes. It is further hypothesized that by editing the NCS of the gene(s) of interest, the stability and half-life of the mRNA transcript can be increased, rather than increasing transcription of the gene, to increase the steady-state level of the mRNA and thus the level of the protein translated from the mRNA. It is, for example, possible that the Nipponbare 2-4 overexpression allele generated in Example 1 has 1) higher expression of OsPsbS1 and/or 2) higher stability of the OsPsbS1 mRNA transcript to generate the novel overexpression phenotype.

The Gao group has previously shown that 5′-UTR editing to remove competing untranslated open reading frames (uORFs) in a gene of interest can result in overexpression phenotypes (Zhang et. al., 2018, Nature Biotech, www[dot]nature[dot]com/articles/nbt.4202). However, this publication specifically emphasizes that those edited genes do not have increased expression but do have increased translation. This is predicted to be due to the loss of ribosomal competition during translation of the gene of interest.

If CRISPR/Cas9 mutagenesis of NCS increases transcript stability instead of gene transcription, this approach and the specific sequence change in Nipponbare 2-4 OsPsbS1 OX are expected to be highly applicable to other species. It is also possible that the specific sequence change is transcript-specific and that therefore a different sequence change will be needed to overexpress other transcripts. The major bottleneck in future gene editing efforts will be in generating similar NCS variation. As the efficiency of prime editing increases and begins to become feasible in planta, known stabilizing sequences such as the one predicted to be within the 5′-UTR of the OsPsbS1 overexpression allele could be introduced into the 5-UTRs of genes of interest.

Enhancers and transcriptional stabilizers can be discovered through in planta/in vitro screening of promising candidates. For example, specific testing of the putative OsPsbS1 OX sequence in a plant expression vector (e.g., in Nicotiana benthamiana) can be used to validate the translatability of that specific sequence across grasses, monocots, and different plant species. Similarly, the experiment in A. thaliana described in Example 3 will identify other promising candidates as well.

What is claimed is:

1. A method of screening for a gain of function mutation in a target gene in a plant comprising:
(a) generating a set of mutations in a non-coding sequence (NCS) of the target gene in a population of plant cells of the plant with one or more RNA-guided nucleic acid modifying enzymes targeting the target gene comprising one or more different guide RNAs;
(b) regenerating the population of plant cells into two or more plants that are hemizygous for the mutation generated;
(c) (1) selfing the two or more plants to generate offspring plants, and (2) optionally screening offspring plants that are homozygous for the mutation for screening in section (d); and
(d) screening the offspring plants from step (c) to identify a gain of function mutation, and optionally further comprising:
(e) selecting a plant with the gain of function mutation, and
(f) sequencing the target gene to identify the gain of function mutation.

2. The method of claim 1, wherein the one or more RNA-guided nucleic acid modifying enzymes is expressed from an expression vector comprising a selectable marker and the screening in step (d) comprises screening for plants lacking the selectable marker.

3. The method of claim 1, wherein the gain of function mutation induces overexpression of the target gene, optionally wherein overexpression of the target gene is in the morning.

4. The method of claim 1, wherein overexpression of the target gene is not constitutive, and/or wherein the plant has a constitutive phenotype.

5. The method of claim 1, wherein the target gene induces a phenotype associated with one or more of photosynthetic efficiency, photoprotection efficiency, non-photochemical quenching, photosynthetic quantum yield, CO₂ fixation, and water use efficiency, and the screening of step (c)(2) comprises screening offspring plants by chlorophyll fluorescence to identify transgene-free plants that are putatively homozygous for the mutation.

6. The method of claim 1, wherein the method does not comprise use of a plant with a hypomorphic allele or a null allele of the target gene.

7. The method of claim 1, wherein the gain of function mutation improves yield, quality, or both in the plant with the gain of function mutation as compared to a plant lacking the gain of function mutation grown under the same conditions.

8. The method of claim 1, wherein the one or more RNA-guided nucleic acid modifying enzymes targeting the target gene comprise two or more different guide RNAs, three or more different guide RNAs, four or more different guide RNAs, five or more different guide RNAs, ten or more different guide RNAs, or twenty or more different guide RNAs, and/or wherein the guide RNAs each target a region of the target gene selected from a promoter region, an upstream regulatory region, a 5′ untranslated region (5′ UTR), a 3′ untranslated region (3′ UTR), an intron, a micro-RNA binding site, an alternative splicing element, and a downstream regulatory element.

9. The method of claim 1, wherein the guide RNAs target at least one region in the target gene that is at least 50% identical, at least 60% identical, at least 70% identical, at least 80% identical, at least 85% identical, at least 90% identical, or at least 95% identical across plant species.

10. The method of claim 1, wherein at least 50% of the set of mutations are in a region of the target gene selected from a promoter region, an upstream regulatory region, a 5′ UTR, a 3′ UTR, an intron, a micro-RNA binding site, an alternative splicing element, and a downstream regulatory element.

11. The method of claim 1, wherein the gain of function mutation is a deletion, inversion, translocation, insertion, transition, transversion, or a combination thereof, and/or wherein the gain of function mutation is an increase in transcription of the target gene, an increase in stability of a mRNA produced from the target gene, an increase in translation of a protein coding region of the mRNA, or a decrease in degradation of the mRNA, in each case as compared to a plant lacking the gain of function mutation grown under the same conditions.

12. The method of claim 1, wherein the one or more RNA-guided nucleic acid modifying enzymes are Cas enzymes, base editors, or prime editors, and wherein the Cas enzymes are selected from the group consisting of Cas9, Cas12, Cas12a, Cas13, Cas14, CasX, and CasY.

13. The method of claim 1, wherein the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant.

14. The method of claim 1, wherein the target gene is selected from a photosystem II subunit S (PsbS) gene, a zeaxanthin epoxidase (ZEP) gene, and a violaxanthin de-epoxidase (VDE) gene.

15. The method of claim 14, wherein the screening comprises assessing one or more of: a photosynthetic efficiency under fluctuating light conditions; a photoprotection efficiency under fluctuating light conditions; an increased rate of induction of non-photochemical quenching (NPQ) under fluctuating light conditions; an increased rate of relaxation of non-photochemical quenching (NPQ) under fluctuating light conditions; an improved quantum yield under fluctuating light conditions, and an improved CO₂ fixation under fluctuating light conditions.

16. The method of claim 14, wherein the target gene is the PsbS gene, and wherein the PsbS gene comprises a sequence selected from the group consisting of SEQ ID NO: 91, SEQ ID NO: 93, SEQ ID NO: 94, SEQ ID NO: 97, and SEQ ID NO: 98, and wherein the one or more different guide RNAs comprise spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90; wherein the target gene is the ZEP gene, and wherein the ZEP gene comprises SEQ ID NO: 92, and wherein the one or more different guide RNAs comprise spacer sequences selected from SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37; or wherein the target gene is the VDE gene, and wherein the VDE gene comprises a sequence selected from the group consisting of SEQ ID NO: 92 and SEQ ID NO: 95, and wherein the one or more different guide RNAs comprise spacer sequences selected from SEQ ID NO: 25, SEQ ID NO: 26, SEQ ID NO: 27, SEQ ID NO: 28, SEQ ID NO: 29, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

17. The method of claim 1, wherein the one or more guide RNAs are introduced using a vector, and wherein the vector comprises two or more gRNA scaffolds comprising SEQ ID NO: 9 and two or more tRNA linkers comprising SEQ ID NO: 10.

18. A method for producing an improved commercial crop plant or crop seed comprising:
(a) selecting a commercial crop plant for improvement, wherein the commercial crop plant comprises a rice (Oryza sativa) plant, a corn (Zea mays) plant, or a cowpea (Vigna unguiculata) plant;
(b) introducing the gain of function mutation identified in the method of claim 1 into at least one cell of the commercial crop plant to generate an improved commercial crop plant cell; and
(c) producing the improved commercial crop plant or crop seed from the improved commercial crop plant cell.

19. An improved commercial crop plant or crop seed comprising a gain of function mutation in a non-coding sequence of a target gene, wherein the target gene induces a phenotype associated with one or more of photosynthetic efficiency, photoprotection efficiency, non-photochemical quenching, photosynthetic quantum yield, CO₂ fixation, and water use efficiency, and wherein the gain of function mutation improves yield, quality, or both in the plant with the gain of function mutation as compared to a plant lacking the gain of function mutation grown under the same conditions,
wherein the target gene is selected from a photosystem II subunit S (PsbS) gene, a zeaxanthin epoxidase (ZEP) gene, and a violaxanthin de-epoxidase (VDE) gene, and/or
wherein the commercial crop plant comprises a rice (Oryza sativa) plant, a corn (Zea mays) plant, or a cowpea (Vigna unguiculata) plant.

20. The improved commercial crop plant or crop seed of claim 19, wherein the target gene is the PsbS gene, and wherein the PsbS gene comprises a sequence selected from the group consisting of SEQ ID NO: 91, SEQ ID NO: 93, SEQ ID NO: 94, SEQ ID NO: 97, and SEQ ID NO: 98.

21. A genetically modified plant comprising an inversion in a cis-regulatory element of a PsbS gene, wherein the inversion increases PsbS gene expression, and wherein increased PsbS gene expression comprises overexpression, increased expression at one or more specific times, increased expression in one or more specific tissues, increased expression at one or more developmental stages, or a combination thereof as compared to a control plant without the inversion in the cis-regulatory element of the PsbS gene.

22. The improved plant of claim 21, wherein the plant is a rice (Oryza sativa) plant, optionally wherein the rice plant is a Oryza sativa ssp. japonica plant.

23. The improved plant of claim 21, wherein the plant or a progenitor thereof was screened for the inversion in the cis-regulatory element of the PsbS gene and increased PsbS gene expression, or wherein the inversion in the cis-regulatory element of the PsbS gene was randomly produced in the plant or a progenitor thereof, optionally wherein the inversion was randomly produced using guide RNAs, and wherein the guide RNAs comprise spacer sequences selected from SEQ ID NO: 7, SEQ ID NO: 8, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 90.

24. The improved plant of claim 21, wherein the increased PsbS expression results in a phenotype associated with one or more of photosynthetic efficiency, photoprotection efficiency, non-photochemical quenching, photosynthetic quantum yield, CO₂ fixation, and water use efficiency, and/or wherein the phenotype is constitutive.

25. The improved plant of claim 21, wherein the increased PsbS gene expression comprises overexpression of PsbS in the morning or overexpression that is not constitutive.


SEQUENCE LISTING (103 sequences)

SEQ ID NO: 1 - DNA, 20 bp, synthetic construct
  misc_feature 1..20: note = Synthetic Construct
  source 1..20: mol_type = other DNA
gcgagacactaaaatacatt

SEQ ID NO: 2 - DNA, 20 bp, synthetic construct
  misc_feature 1..20: note = Synthetic Construct
  source 1..20: mol_type = other DNA
tcttgttcctggatgtaatt

SEQ ID NO: 3 - DNA, 20 bp, synthetic construct
  misc_feature 1..20: note = Synthetic Construct
  source 1..20: mol_type = other DNA
agattcaggagtaacaaaaa

SEQ ID NO: 4 - DNA, 20 bp, synthetic construct
  misc_feature 1..20: note = Synthetic Construct
  source 1..20: mol_type = other DNA
tgttgcatgtggtccgtcga

SEQ ID NO: 5 - DNA, 20 bp, synthetic construct
  misc_feature 1..20: note = Synthetic Construct
  source 1..20: mol_type = other DNA
cacaaaaaagtacgggaatg

SEQ ID NO: 6 - DNA, 20 bp, synthetic construct
  misc_feature 1..20: note = Synthetic Construct
  source 1..20: mol_type = other DNA
taccaaccacctgctcttct

SEQ ID NO: 7 - DNA, 20 bp, synthetic construct
  misc_feature 1..20: note = Synthetic Construct
  source 1..20: mol_type = other DNA
gtctccccgaatcccttcta

SEQ ID NO: 8 - DNA, 20 bp, synthetic construct
  misc_feature 1..20: note = Synthetic Construct
  source 1..20: mol_type = other DNA
ctacgcctcccacccgccac

SEQ ID NO: 9 - DNA, 76 bp, synthetic construct
  misc_feature 1..76: note = Synthetic Construct
  source 1..76: mol_type = other DNA
gttttagagctagaaatagcaagttaaaataaggctagtccgttatcaacttgaaaaagtggcaccgagtcggtgc

SEQ ID NO: 10 - DNA, 77 bp, synthetic construct
  misc_feature 1..77: note = Synthetic Construct
  source 1..77: mol_type = other DNA
aacaaagcaccagtggtctagtggtagaatagtaccctgccacggtacagacccgggttcgattcccggctggtgca

SEQ ID NO: 11 - DNA, 27 bp, synthetic construct
  misc_feature 1..27: note = Synthetic Construct
  source 1..27: mol_type = other DNA
agacagaggtatgtcaatgtgttattg

SEQ ID NO: 12 - DNA, 20 bp, synthetic construct
  misc_feature 1..20: note = Synthetic Construct
  source 1..20: mol_type = other DNA
tggtaggccatttgctcttt

SEQ ID NO: 13 - DNA, 20 bp, synthetic construct
  misc_feature 1..20: note = Synthetic Construct
  source 1..20: mol_type = other DNA
aaagagcaaatggcctacca

SEQ ID NO: 14 - DNA, 21 bp, synthetic construct
  misc_feature 1..21: note = Synthetic Construct
  source 1..21: mol_type = other DNA
catccatccaaattccaacct

SEQ ID NO: 15 - DNA, 20 bp, synthetic construct
  misc_feature 1..20: note = Synthetic Construct
  source 1..20: mol_type = other DNA
cgtgtctcccacgtcttctt

SEQ ID NO: 16 - DNA, 20 bp, synthetic construct
  misc_feature 1..20: note = Synthetic Construct
  source 1..20: mol_type = other DNA
gagcaaacactcaggcacaa

SEQ ID NO: 17 - DNA, 20 bp, synthetic construct
  misc_feature 1..20: note = Synthetic Construct
  source 1..20: mol_type = other DNA
tgcaccctagggctgtcaac

SEQ ID NO: 18 - DNA, 22 bp, synthetic construct
  misc_feature 1..22: note = Synthetic Construct
  source 1..22: mol_type = other DNA
ggcgagtgacgctctagttctt

SEQ ID NO: 19 - DNA, 21 bp, synthetic construct
  misc_feature 1..21: note = Synthetic Construct
  source 1..21: mol_type = other DNA
accacttcgaccgccactact

SEQ ID NO: 20 - DNA, 19 bp, synthetic construct
  misc_feature 1..19: note = Synthetic Construct
  source 1..19: mol_type = other DNA
acgcctaagcctgctggtt

SEQ ID NO: 21 - DNA, 24 bp, synthetic construct
  misc_feature 1..24: note = Synthetic Construct
  source 1..24: mol_type = other DNA
tttcactcttggtgtgaagcagat

SEQ ID NO: 22 - DNA, 25 bp, synthetic construct
  misc_feature 1..25: note = Synthetic Construct
  source 1..25: mol_type = other DNA
gacttccttcacgatttcatcgtaa

SEQ ID NO: 23 - DNA, 18 bp, synthetic construct
  misc_feature 1..18: note = Synthetic Construct
  source 1..18: mol_type = other DNA
ctgttcggcaggtccaag

SEQ ID NO: 24 - DNA, 18 bp, synthetic construct
  misc_feature 1..18: note = Synthetic Construct
  source 1..18: mol_type = other DNA
caaacccgagcatggcga

SEQ ID NO: 25 - DNA, 20 bp, synthetic construct
  misc_feature 1..20: note = Synthetic Construct
  source 1..20: mol_type = other DNA
gccacgtcgggccaaacggg

SEQ ID NO: 26 - DNA, 20 bp, synthetic construct
  misc_feature 1..20: note = Synthetic Construct
  source 1..20: mol_type = other DNA
acaacatatggaaaaatcgg

SEQ ID NO: 27 - DNA, 20 bp, synthetic construct
  misc_feature 1..20: note = Synthetic Construct
  source 1..20: mol_type = other DNA
aatgggccgggccaaggcct

SEQ ID NO: 28 - DNA, 20 bp, synthetic construct
  misc_feature 1..20: note = Synthetic Construct
  source 1..20: mol_type = other DNA
agccaagccaagcccctccg

SEQ ID NO: 29 - DNA, 20 bp, synthetic construct
  misc_feature 1..20: note = Synthetic Construct
  source 1..20: mol_type = other DNA
gggatcgagagctcgagcag

SEQ ID NO: 30 - DNA, 20 bp, synthetic construct
  misc_feature 1..20: note = Synthetic Construct
  source 1..20: mol_type = other DNA
ccaaacctttacaaaccgct

SEQ ID NO: 31 - DNA, 20 bp, synthetic construct
  misc_feature 1..20: note = Synthetic Construct
  source 1..20: mol_type = other DNA
ttaaccttataggttgaaat

SEQ ID NO: 32 - DNA, 20 bp, synthetic construct
  misc_feature 1..20: note = Synthetic Construct
  source 1..20: mol_type = other DNA
gggagtatatacctttaggg

SEQ ID NO: 33 - DNA, 20 bp, synthetic construct
  misc_feature 1..20: note = Synthetic Construct
  source 1..20: mol_type = other DNA
ttctcaccgttaattctaaa

SEQ ID NO: 34 - DNA, 20 bp, synthetic construct
  misc_feature 1..20: note = Synthetic Construct
  source 1..20: mol_type = other DNA
ttgtggaacacagttcatgg

SEQ ID NO: 35 - DNA, 20 bp, synthetic construct
  misc_feature 1..20: note = Synthetic Construct
  source 1..20: mol_type = other DNA
acacatggttagccacccag

SEQ ID NO: 36 - DNA, 20 bp, synthetic construct
  misc_feature 1..20: note = Synthetic Construct
  source 1..20: mol_type = other DNA
tttgggctgcggtcgtggaa

SEQ ID NO: 37 - DNA, 20 bp, synthetic construct
  misc_feature 1..20: note = Synthetic Construct
  source 1..20: mol_type = other DNA
gtgtagcggagacggagcaa

SEQ ID NO: 38 - DNA, 20 bp, synthetic construct
  misc_feature 1..20: note = Synthetic Construct
  source 1..20: mol_type = other DNA
agttgcccaaaaaaagaaga

SEQ ID NO: 39 - DNA, 20 bp, synthetic construct
  misc_feature 1..20: note = Synthetic Construct
  source 1..20: mol_type = other DNA
gcttgtcatgatgtgagatg

SEQ ID NO: 40 - DNA, 20 bp, synthetic construct
  misc_feature 1..20: note = Synthetic Construct
  source 1..20: mol_type = other DNA
ttatttaattactatcgcac

SEQ ID NO: 41 - DNA, 20 bp, synthetic construct
  misc_feature 1..20: note = Synthetic Construct
  source 1..20: mol_type = other DNA
gtaattctttgactatttca

SEQ ID NO: 42 - DNA, 20 bp, synthetic construct
  misc_feature 1..20: note = Synthetic Construct
  source 1..20: mol_type = other DNA
gcagcgtttgcggttggtag

SEQ ID NO: 43 - DNA, 20 bp, synthetic construct
  misc_feature 1..20: note = Synthetic Construct
  source 1..20: mol_type = other DNA
gagccgggcacaaacacaag

SEQ ID NO: 44 - DNA, 20 bp, synthetic construct
  misc_feature 1..20: note = Synthetic Construct
  source 1..20: mol_type = other DNA
atagaagattcacgtcaaaa

SEQ ID NO: 45 - DNA, 20 bp, synthetic construct
  misc_feature 1..20: note = Synthetic Construct
  source 1..20: mol_type = other DNA
gagagtatggaaaggacaat

SEQ ID NO: 46 - DNA, 20 bp, synthetic construct
  misc_feature 1..20: note = Synthetic Construct
  source 1..20: mol_type = other DNA
tgaagagattaaaagatata

SEQ ID NO: 47 - DNA, 20 bp, synthetic construct
  misc_feature 1..20: note = Synthetic Construct
  source 1..20: mol_type = other DNA
ttctttctgaggatgagaga

SEQ ID NO: 48 - DNA, 20 bp, synthetic construct
  misc_feature 1..20: note = Synthetic Construct
  source 1..20: mol_type = other DNA
tactctttacttgtcccaca

SEQ ID NO: 49 - DNA, 20 bp, synthetic construct
  misc_feature 1..20: note = Synthetic Construct
  source 1..20: mol_type = other DNA
agttagtgtatttcatagag

SEQ ID NO: 50 - DNA, 20 bp, synthetic construct
  misc_feature 1..20: note = Synthetic Construct
  source 1..20: mol_type = other DNA
cctaagtgtagtgtccggat

SEQ ID NO: 51 - DNA, 20 bp, synthetic construct
  misc_feature 1..20: note = Synthetic Construct
  source 1..20: mol_type = other DNA
tttcccccatgtaagcacat

SEQ ID NO: 52 - DNA, 20 bp, synthetic construct
  misc_feature 1..20: note = Synthetic Construct
  source 1..20: mol_type = other DNA
tctcttcatgttgagacaaa

SEQ ID NO: 53 - DNA, 20 bp, synthetic construct
  misc_feature 1..20: note = Synthetic Construct
  source 1..20: mol_type = other DNA
aattccaagttaaaaacaaa

SEQ ID NO: 54 - DNA, 20 bp, synthetic construct
  misc_feature 1..20: note = Synthetic Construct
  source 1..20: mol_type = other DNA
tatcaatcacctagtcactt

SEQ ID NO: 55 - DNA, 20 bp, synthetic construct
  misc_feature 1..20: note = Synthetic Construct
  source 1..20: mol_type = other DNA
ggtggagaaaacaaccgcct

SEQ ID NO: 56 - DNA, 20 bp, synthetic construct
  misc_feature 1..20: note = Synthetic Construct
  source 1..20: mol_type = other DNA
tttgttcacacaccacacga

SEQ ID NO: 57 - DNA, 20 bp, synthetic construct
  misc_feature 1..20: note = Synthetic Construct
  source 1..20: mol_type = other DNA
aacaaaaactgaaagctccg

SEQ ID NO: 58 - DNA, 20 bp, synthetic construct
  misc_feature 1..20: note = Synthetic Construct
  source 1..20: mol_type = other DNA
actcaggtattgcttggtgt

SEQ ID NO: 59 - DNA, 20 bp, synthetic construct
  misc_feature 1..20: note = Synthetic Construct
  source 1..20: mol_type = other DNA
aaccaggaatcataaaacgt

SEQ ID NO: 60 - DNA, 20 bp, synthetic construct
  misc_feature 1..20: note = Synthetic Construct
  source 1..20: mol_type = other DNA
tagtgtccccccaccaaaac

SEQ ID NO: 61 - DNA, 20 bp, synthetic construct
  misc_feature 1..20: note = Synthetic Construct
  source 1..20: mol_type = other DNA
gagggaaatatataaattga

SEQ ID NO: 62 - DNA, 20 bp, synthetic construct
  misc_feature 1..20: note = Synthetic Construct
  source 1..20: mol_type = other DNA
ataattataaacaaaatcat

SEQ ID NO: 63 - DNA, 20 bp, synthetic construct
  misc_feature 1..20: note = Synthetic Construct
  source 1..20: mol_type = other DNA
acatcttgtatccacccacg

SEQ ID NO: 64 - DNA, 20 bp, synthetic construct
  misc_feature 1..20: note = Synthetic Construct
  source 1..20: mol_type = other DNA
tatagtttgtacaacaatgg

SEQ ID NO: 65 - DNA, 20 bp, synthetic construct
  misc_feature 1..20: note = Synthetic Construct
  source 1..20: mol_type = other DNA
aattggatacagaaaacaca

SEQ ID NO: 66 - DNA, 20 bp, synthetic construct
  misc_feature 1..20: note = Synthetic Construct
  source 1..20: mol_type = other DNA
aagaaaggttggaatttgga

SEQ ID NO: 67 - DNA, 20 bp, synthetic construct
  misc_feature 1..20: note = Synthetic Construct
  source 1..20: mol_type = other DNA
ttaacggaagcactttaccc

SEQ ID NO: 68 - DNA, 20 bp, synthetic construct
  misc_feature 1..20: note = Synthetic Construct
  source 1..20: mol_type = other DNA
tctttgttattgctgcagga

SEQ ID NO: 69 - DNA, 20 bp, synthetic construct
  misc_feature 1..20: note = Synthetic Construct
  source 1..20: mol_type = other DNA
agtgcgagtagcattctagt

SEQ ID NO: 70 - DNA, 20 bp, synthetic construct
  misc_feature 1..20: note = Synthetic Construct
  source 1..20: mol_type = other DNA
tatatagtgtggcaagatgg

SEQ ID NO: 71 - DNA, 20 bp, synthetic construct
  misc_feature 1..20: note = Synthetic Construct
  source 1..20: mol_type = other DNA
tgagcgcttttgcatcaggt

SEQ ID NO: 72 - DNA, 20 bp, synthetic construct
  misc_feature 1..20: note = Synthetic Construct
  source 1..20: mol_type = other DNA
atgcgcaagcacaacaatgg

SEQ ID NO: 73 - DNA, 20 bp, synthetic construct
  misc_feature 1..20: note = Synthetic Construct
  source 1..20: mol_type = other DNA
tgagatttagctacttgctg

SEQ ID NO: 74 - DNA, 20 bp, synthetic construct
  misc_feature 1..20: note = Synthetic Construct
  source 1..20: mol_type = other DNA
ggtagtcctaggcgagcgcg

SEQ ID NO: 75 - DNA, 20 bp, synthetic construct
  misc_feature 1..20: note = Synthetic Construct
  source 1..20: mol_type = other DNA
gcacagagacggataaagag

SEQ ID NO: 76 - DNA, 20 bp, synthetic construct
  misc_feature 1..20: note = Synthetic Construct
  source 1..20: mol_type = other DNA
gcatgcgtgcacataacaca

SEQ ID NO: 77 - DNA, 20 bp, synthetic construct
  misc_feature 1..20: note = Synthetic Construct
  source 1..20: mol_type = other DNA
caacgacttataatttcgga

SEQ ID NO: 78 - DNA, 20 bp, synthetic construct
  misc_feature 1..20: note = Synthetic Construct
  source 1..20: mol_type = other DNA
tttataatttagacttggag

SEQ ID NO: 79 - DNA, 20 bp, synthetic construct
  misc_feature 1..20: note = Synthetic Construct
  source 1..20: mol_type = other DNA
gggtggaagaaaattccaaa

SEQ ID NO: 80 - DNA, 20 bp, synthetic construct
  misc_feature 1..20: note = Synthetic Construct
  source 1..20: mol_type = other DNA
tataataatgatggtgcctg

SEQ ID NO: 81 - DNA, 20 bp, synthetic construct
  misc_feature 1..20: note = Synthetic Construct
  source 1..20: mol_type = other DNA
acgcacacgactcatgtccg

SEQ ID NO: 82 - DNA, 20 bp, synthetic construct
  misc_feature 1..20: note = Synthetic Construct
  source 1..20: mol_type = other DNA
tactactagctaatatgcca

SEQ ID NO: 83 - DNA, 20 bp, synthetic construct
  misc_feature 1..20: note = Synthetic Construct
  source 1..20: mol_type = other DNA
gagtgtactaagtttgaggc

SEQ ID NO: 84 - DNA, 20 bp, synthetic construct
  misc_feature 1..20: note = Synthetic Construct
  source 1..20: mol_type = other DNA
caccatctataatttaacaa

SEQ ID NO: 85 - DNA, 20 bp, synthetic construct
  misc_feature 1..20: note = Synthetic Construct
  source 1..20: mol_type = other DNA
cattctgaaccaaaccaccg

SEQ ID NO: 86 - DNA, 20 bp, synthetic construct
  misc_feature 1..20: note = Synthetic Construct
  source 1..20: mol_type = other DNA
gtttcatttacataaaagaa

SEQ ID NO: 87 - DNA, 20 bp, synthetic construct
  misc_feature 1..20: note = Synthetic Construct
  source 1..20: mol_type = other DNA
ccttgtaatttaataaacag

SEQ ID NO: 88 - DNA, 20 bp, synthetic construct
  misc_feature 1..20: note = Synthetic Construct
  source 1..20: mol_type = other DNA
atagaagagtaacatttgtt

SEQ ID NO: 89 - DNA, 20 bp, synthetic construct
  misc_feature 1..20: note = Synthetic Construct
  source 1..20: mol_type = other DNA
gcattgatgatagataagga

SEQ ID NO: 90 - DNA, 20 bp, synthetic construct
  misc_feature 1..20: note = Synthetic Construct
  source 1..20: mol_type = other DNA
gttgcgccgacaccaagcaa

SEQ ID NO: 91 - DNA, 4688 bp, Arabidopsis thaliana
  source 1..4688: mol_type = unassigned DNA
atatatgtgtttcctagtaagttaactgtatttctttgtgacatattatgttatgtgtaccatgttatttggctatttgctatgataatgtataaattatcacatagcgtcctagcgaaattaataaaatctatactcgaaaatcattaatctgaaagttcattaacagatacacgccaacataaaactataacatgttaaagagaaaaatactattttctaattgattaaataaaatgtaaaaacatccaaaaatacatgtcacattattgttattttacttattaggtttgttcccaaagtatatgcctaagcccacttgttttgtaataacgtgaaatgagcagttcgtgttcaaacacgtcttacatggagacaattaattaaacaccacttacttgttactacaaatatactctttcggagtgttgcgagcaatttataagtcgcacatatttttgactcgaatcgagtcaatcaggttttctcaaactattagggattttatgtaatatttttttttaaaaggtatattgatgtgagttgcccaaaaaaagaagaaggtgtgattgatgtgaaattgttttcaaaataggggtttaatgtatgtacatataattaaaattaaaatgaaagtttatgatatttaaatattatatgtccattttgaatatatatgatgattgttcaattgatacaatttaaattacaaaataatgaactttttagtagaagacaatgttttgtgtttaaattataactttaatgagactcatagaaagaaaacaaatatgcctaaacccgcttgtcatgatgtgagatgaggtgttcgtgtccaaagttcaaacacgtctctcatcgagaaaactaattaaacaccaaaaattattcctttctgagtgttgtgcgagtatttgtagtttcaaatgttttttactcgattcaagtttttaccgtccctaactattttaaggttataagcgggtgcggttgcggttacgggagtttgcggaaacgggcgatcacggttttaagcgttatcaagcattttgaacgactggttgagcgtttgataataggtgcgtttgcgggagttttatgactgataaaccagccggtgcgatagtaattaaataaaaaataaccaaaataaatataatatattactattaaaagaaataaaaatataatatattagaaaaattgattttctgaccaaataaaaatacaatatatcatctcgtccgactaaaacattttctaatcgagaaaacaaatcgaaacatgtattcaatcaaaaaactcaaaagaatacaaaataaaataaaaacaatgatgccatgaaatagtcaaagaattacactagctaatgagtgaataaaatgatagatgatacttatgtttgattcttgattttgattttcatcggatgccataattgagagaatagagttacaatatattgattagggttttctaataaaaataagatatataaaagattagattattttaattatagtgattagggtttgggtggaaaccacaattattttaattaactaaaaatagattatttttttatttttaatctaaccgctaccgcccgcaaccgcaaacgcttgcgggaaggagcttttgaaaattggcaattttgagcggtccaaagcgatatctagcggtttctatgattggttccaatcgctaataaccgctaccaaccgcaaacgctgcgtttatgggtggtagagggagaaccaatcagcccttaacaaaaccgaaaaaactaaagatttgaacctaagtgtcgaagattctttcggaccagtctttaccactagactacctaacaaatacgaaaatatgaagccaaaattgtttttatactatttgcgaccgtaagtttgggtttagcccgcttgtgtttgtgcccggctctggctatattaaaaaagaattagatatttttcaaatataatattttttacaaaatatgtaattgatgtgaattttttcaatataggtgttcttatttatatacatattataaaaaaaattaagccatttcgtttttgtaatatctaatgtaccagttccattagtatgtactttgctagtataacatacccttttgacgtgaatcttctatgagtatgtttctttagttttgaccagacacaattcatttacatcaatacataaattggattttagttccaaaatatgattttcttgaaagaaatattttatatttttattagaaggagttattagttcgtcataaatggttctttgaatatgacatttattttgtttttttgtttactgtctaaataagtataacttttaaaaagtgtatctaaattttttaaaaactattatattgatagatttatttttctccacaaaaacgttatctccagttcaccacatagagagtatggaaaggacaattgggagacgtggacgcactttaagcccatcaatagatttcccttatatcttttaatctcttcacttccacaacacaacacaaaacacatcactgatccttctctcatcctcagaaagaatggctcaaaccatgctgcttacttcaggcgtcaccgccggccattttttgaggaacaagagccctttggctcagcccaaagttcaccatctcttcctctctggaaactctccggttgcactaccatctaggagacaatcattcgttcctctcgctctcttcaaacccaaaaccaaagctgctcctaaaaaggttttcacatactctttacttgtcccacaaggaacatttcttgtgtgtttgatggtaatgttaagattttcatatacattttaggttgagaagccgaagagcaaggttgaggatggcatctttggaacgtctggtgggattggtttcacaaaggcgaatgagctattcgttggtcgtgttgctatgatcggtttcgctgtaagtcccctctatgaaatacactaacttatcttccaagaaagaagctttagtatgaaagctgtgtttctatgcataggcatcgttgcttggtgaggcgttgacgggaaaagggatattagctcagctgaatctggagacagggataccgatttacgaagcagagccattgcttctcttcttcatcttgttcactctgttgggagccattggagctctcggagacagaggaaaattcgtcgacgatcctcccaccgggctcgagaaagccgtcattcctcccggcaaaaacgtccgatctgccctcggtctcaaagaacaaggtatctctcaacatttctatttctatctttgttacaatgacattgctctgaacaaccaatgcaaataatataacctgatagagtactatagtaactagtataactgaattggcctaagtgtagtgtccggatcggctttatgtttatttatcgttatcctaagatacaaatgtgagacaaaagatactagcatggaactgtcaatctccaaaaataatctctaaatgtttatttatttatcgttggcctgatagattatttatcgttatcaatttctatctttttcccccatgtaagcacattggttcctgaatggctttctctagcagagagtctcttcccgttaacttaaataaaatagtgtttctgaaaagaaaaaaagatacaaatatgagattacttaaagagttgcttggtctaatgttttgaataaatttgaatatgtttgttgttttgttgtgtgaaggtccattgtttgggttcacgaaggcgaacgagttattcgtaggaagattggcacagttgggaatagcattttcactgataggagagattattaccgggaaaggagcattagctcaactcaacattgagaccggtataccaattcaagatatcgaaccacttgtcctcttaaacgttgctttcttcttcttcgctgccattaatcctggtaatggaaaattcatcaccgatgatggtgaagaaagctaaattatcatgtacttaaatttagtagagagtgtgtgaccttctcttcatgttgagacaaaaggaaatggacagcttaaattgttgtaatacttatatccttttgtttttaacttggaattttctgattatatatagttttattacacctaagtgactaggtgattgatatcatctagcatctcttaacagaaaaaaactgtggaattagaacagcttcatcatagtttttttttttgataaatcaaacagaatttaaaactaatatgttaaagtacatgttaggaacttatttgattccaaaatctagattataaaattaatttggtaaatcatgaaggagccgttgtttttgctctagtggttccccaaaaatcagaacataatggcaagtgtttatggccgcgcgtaccttcacaagatagtttgcaaaagtgaaggtagcgtctcccctagagtgattttctccattcgtctggtgtacctggctcgtacaggccgtctctgggggtggcaaggtaggcaaatgcccaaggttactaatcaatttcaaaaaaattgttaagactaaaaggccccaatttttccaaaaaaaaacatttactaagagattttattttattttgagttttgccataggtctcataatgaattgagccgggatctaaagcatacactgcaagagtaaatat

SEQ ID NO: 92 - DNA, 5608 bp, Arabidopsis thaliana
  source 1..5608: mol_type = unassigned DNA
ttattcttagtatttggtagctgtagctgttgtaatttgaagtccacttgataacttccttgtatttgtaggcgaagtatttagaagctttagcctctgagacacttctcacaagcgatgaagcagtagttgtagcagcagcagctgaagcagtcgcccttgcaagagctgctgtcaaagttgccaaagatgcaacattatttaagaacagtaacaacacgaacctattaacttcgtcaacggccgacaaacgctccaagtgggaccagtttactgagaaggaacgtgctggcatattggggcatctagcggtttcggacaatggaattgtgagtgataaaatcactgcatctgcctctaacaaagagtctattggtgatttagaatcagaaaaacaagaagaagttgagcttctggaggagcaaccttcagtgagtttagctgtgagatctacacgtcaaactgaaaggaaagctcggagggcaaaagggttagagaaaactgcatcaggtattccgtctgtgaagactggttcgagccctaaaaagaaacgtcttgttgcgcaggaagttgatcataatgatcctttgcgttatctaagaatgacaacaagcagttccaagcttctcactgtcagagaagaacatgagctgtcggcaggaatacaggtttgtcaccaatattccaaattctaaactaaccacctccctttttctacatcgcgtctgagacatcacaaacactcacccttcgtaggactctagattctaggcgaaaatgatgtctacaaaatctttagatgtttgagaatgacgaaattagtggttagtggtagaatccaagatcgcttctttgtgctttctagccaatatatcatctgaagttctgtgaaagctaattatcctgacatgcttgtcttaaattgaattgtcaccttttttgttcttattatgtagccattcaccaactgtttagtgttgatagaaaatgggaaacgtatgttcagctttggaagaatcaacattacttgtgaacgttatttcttcttctctgtaatttttcttatctttctttattgtggttttgatcacacactttggtttctttgtttaggaccttctgaagttagaaagacttcaaacagagcttacagagcgtagtggacgtcagccaacctttgcgcagtgggcttctgctgctggagtcgatcagaaatcattaaggcaacgtatacatcatggcacactatgcaaagacaaaatgatcaaaagcaacattcgactcgttatttcgattgcaaagaattatcaaggagctgggatgaacctccaagatcttgtccaggtttgagcttttctctccttatttttttttgtaataacacatctgtaagcagtagtgatgaaagtgatagatattgaacaccttgctcaaatattttcgaaatggtcacaggaagggtgcagagggcttgtgaggggagcagagaagtttgatgctacaaagggttttaaattttcgacttacgcgcattggtggatcaagcaagctgtgcggaagtctctctctgatcagtccagaatgataagattgcctgtaagttatcccaaatgtttttcttttttacttgtttaaacaaaaggtttgggattatgcctcaaattttaagggatgattggcagtttcacatggtggaagcaacatatagggtgaaagaggcacgaaagcaactgtacagtgaaaccggtaagcacccaaagaacgaagaaattgcagaggcaacagggctgtcgatgaagagactcatggcggttctactctctcctaaacctccgaggtcgctagaccagaaaatcggaatgaatcaaaacctcaaaccttcggttggtaccacctgacacaacacacaccatacaacatagacttgttgtttgtctaaaaatagtaaataatatgagatgggtttgtaatgtaaaaatatgcaggaagtgatagcagatccagaagcagtaacgtcagaagatatactgataaaggaattcatgaggcaggacttggacaaagtgttggactcgttgggtacaagggagaaacaagtgatacgttggagatttgggatggaggatgggagaatgaagacgttgcaagagataggagagatgatgggagtgagcagggagagagtaagacagatagagtcatctgcattcaggaaactaaagaacaagaagagaaacaaccatttgcagcaatacttggttgcacaatcataattctttttctcttcaatatggaatgttttgtgcatagaacccacccacctgagcataacaagcagtaaattattactttttagcatttacgattcttgtgacaactactaaatcgaaaaaattattttaaaactaaaaagaaattgttgtctgaaaaacataagtgatatacaacaataaataaatatctcttataagaatttgggaaaaaaaaaatccaaagattagagatatttgggtggagaaaacaaccgcctaggacaaaaaaaaattgtgggtagaggaggaggaggaggctttgttcacacaccacacgacggaacattctcttccgagtcttatccaaagcaaaatcatattttgcttaaatatcttcttctccggctctgtctctgtctctcttcttcccgcggagctttcagtttttgtttggtaacatcgtttctctttctcactaactctgatatcatattatcaatttttccttttcaacacccaaaatcacaatctttcagctttattctgtttttccttgcttgagaattgtgttcgattcatgtctacgaactgaaatcacaaattttgccaacgttttatgattcctggttccgtatttttttttttgtaatcaatagttagctcatttcgaataatcaaactcaattttattgttcatgatttgttatctgggataaaagagagtaatgtgctaattatgtatactcaggtattgcttggtgtggggaagattagatagtgtgaagaatggcagtagctacacattgtttcacttcaccttgtcatgaccgtattcgatttttctcaagtgatgatggtattggtaggcttggcattacaagaaagaggatcaatggcactttcttgctcaagattttacctccaatccaaagtgctgatctcagaacaactggtgggagatcctcacgtcctttatctgcattcaggtcaggattctctaaggtaatcaatcatactagtcttgtgtttcaatacaaaatttggtatttcgatgatctcattgcttttgatcatgtcaggggatatttgacattgtgccattaccatcaaagaatgagctgaaagagctgaccgctccgctgttgctaaaactcgtgggtgttttagcttgcgcgttccttattgttccatctgcagatgcagttgatgcacttaaaacttgtgcatgcttattgaagggatgcaggtaactttccttcgtttggattctgcatatatagtgtccccccaccaaaaccggttttattttcattgtgattgtgatgctataagtgttttgaattgtatagctccatactcttcaattgttgagaatgttaaagctagatgaagttactgtgtctgtctatgatagagctgaaatcagttgatgatatctttctgatttttttgccgtcaatttatatatttccctccttgtctgaccatatggaagcccaattatctgttaggatagaactcgcaaagtgcattgccaaccctgcctgtgcagccaatgtcgcgtgccttcagacctgcaataaccgtccagatgaaaccgagtgccaggttataatttattttgatatatcattttatgaggcagagatattgcttgcttatgaaagaagaccaatgattttgtttataattatgtgcttatattatatcagattaaatgtggggatctgtttgagaacagtgttgttgatgagttcaacgagtgtgctgtgtcgagaaaaaagtgtgttcctagaaaatctgatctcggagaatttcctgccccagacccttctgttcttgtacagaacttcaacatctcggactttaacgggaagtggtacattacaagtggcttgaatccaacctttgatgccttcgactgccagctgcatgagttccacacagaaggtgacaacaagcttgttggaaacatctcttggagaataaagaccctagacagtggattctttactaggtcagccgtacaaaaattcgtgcaagatcctaaccaacctggtgttctctacaatcatgacaacgagtaccttcactatcaagatgactggtaataacatcttgtatccacccacgagggagagtcttgatagtgctgctattcttctttggcctaacaagctttcatatgatttttgcaggtatatcctgtcatcaaagatagagaataaacctgaagactatatatttgtatactaccgtgggcgaaacgatgcttgggatggatatggtggtgcagttgtatacacgagaagttctgtattacccaatagcattataccagaactcgaaaaagcagcaaaaagcataggcagagacttcagcacattcattagaacggataacacatgtggtcctgaacctgcgctcgtggagagaattgagaagacagtggaagaaggtgaaaggataatcgtaaaagaggttgaagagatagaagaagaggtagagaaggaagtggagaaggtcggtaggactgagatgaccttgttccagagattggctgaaggatttaatgaactgaagcaagacgaggagaatttcgtgagagagttaagtaaagaagagatggagtttttggatgagatcaaaatggaagcaagtgaggttgaaaaattgtttgggaaagctttgccaatcaggaaggtcaggtagaaacaagaaccaccattgttgtacaaactatattatacatactgtgttcggttcatataaagtaatatttttgtacacagtcatcatcattccataacaattggatacagaaaacacaaggattaaacatcctttgagcgaaacaaacaacgattggtaaacatagtacctcaagaagagctgaaagtgttaacaatggggatgacaacatttagaatgatcaagaggattaagtcaactgggtatctttgtaaacattcaccatttcattgaaattggatacaagacacgaaaggattaagacatccttgaagaacacaaaaggattaagacatccattggtacaaagaagagcgaaacaaaacaaaaggagtgaacatagccagtatctcaagaagagctgaaactggtaatgatggggatgacaacaatgagaatgatcaaaagcagcacaatgataccaatgcacatccattttctgctgtttctctgatgactctttgcagtcttcagctcattagctccatcagccacgtaatggctcgcattaatcacatgatgctcgatctcgtccatctgttcaccttgcgattcaaccatcacagccatatcaagaaacacttggtgaagctccaacagactct

SEQ ID NO: 93 - DNA, 4186 bp, Oryza sativa
  source 1..4186: mol_type = unassigned DNA
agacagaggtatgtcaatgtgttattggcctctacgttcggtagaaactagtaatgatgtactatattatgtgttctgaactcattataagatctctaccagcaaatgattctttgctattgtgttactatttgtatattgcgtttcatttatcatcatatgcagacaacctggatacaacatcgttgctcatattacaggtagaaaacgaacaaaattaaatggcattgtgtgccatcccgttttgttttaaagcttcgcctaaatcctaatgtattttagtgtctcgctgtgattgaattgtttccactaactgttgcatcatttgcactcttgtagtacttcatgtacactcttctctttaaaacgagactgaaatatacactgatgtatcatatccttttctcctgaatcttgttgactcaaagtagttcattctatcacgagtacttatcttctgcaagaacatcttttcatttttcttgttcctggatgtaatttggagctaaattgttttgagtactactaatatttaccaggaaccatttttgttactcctgaatctggtgacttgggggtaaattgcatacattagcgataaaaaaaagtaggtcccaccgggattcgaacccaggtcgccagattcaaagtctggagtgctaaccactacaccatggaaccggttgttgcatgtggtccgtcgatggtgatacttctgtcttttactgaagatcagtaacaaagggaagtatactagttgaaacccacatgttcatttgtgggcatccatctgctaggcacttcaattaaacaaaaacacgggaatatccaacgaatcctcttctccacattcccgtacttttttgtgctgaatcgaatttcctgacgccatataaaaaaacgaatttcctgacatatctcaggaaaaaaaaaaggtaatactcgagagtcgataacaccctcctagactcctacggcgtggcacacagttcactaaaatccaggcaacacaagtacacaacacgcagagtcacacagatgcttggagttggagcacttttccagtgacgcgtgcagtttaccaaccacctgctcttctcggccagttcgctgcggacctccataacccaagcggagatgatcgcgcaccgtgtcagcgcgtcgcggccatagaagggattcggggagacgtggccgcccgcgcgcccgtcgcgcaccctatcctcgccgcgcgcccgccggccaatcgccacccctgaatccacagccacgccacgcggcccatgcctacgcctcccacccgccacaggatctccctctcacgcgcgcgcgatctcataaaaacctcgccacctcgatcggtgaggtagcacgaggcggcctgagctgaacaccgcatcagcagcagcaagcagctaatcgcgcacgtacgcacaccgcggcccggggcgagatggcgcagtcgatgctggtgtcgggagccaatggcacggtggccgccgcgagcaccagcaggctgcagcccgtgcggccgacgccgttctcgcggctcgtgctgtcgcagccgtcgtcgtcgctggggcgcgcggtgtccgtcaagacggtggcgctgttcggcaggtccaagaccaaggccgcgcccgcaagaaaggttggaatttggatggatgaatgcaagctaaactaagctaagctaagctcgcgtttgttttagtcttatgtttgatggtgctaactattttgtaacgtgtcaacgtgtgtatgtaggctgagccgaagccaaagttcaagaccgaggacggcatcttcggcacgtccggtgggatcggtttcaccaaggagaacgagctgttcgtcgggcgtgtcgccatgctcgggtttgccgtaagtagacctttcttggccttctagttatttggtatgcatcatttcttgccatcttggtacgtacctgtgcgttcgggtttgggttagctagctagcaagagcacaagcttaaacgtcaaaagttttgtggttaacggaagcactttacccgggagcgtcatcctttattctataaagggtcacatgttgcataggtagatttgcttccaactactttttttaaaaaaaaattatagtttttgtcatatattttactttgaattttctgtgcacataaaaatgctatatttctccttagcaaatattgctacttttattttatttttcagtccgagttttgacagcgacattgtttaatctagaccagttagcaactcacatgtataccacacacataactaatgaattatctggatttagtaatataggtattgtaggttttaaaaaattcgtctttcgagcagatatattgtttaatgagtaaattttagagaactataaatataaatttagtgactaaattatcagcttgttgtaacgttagtgacatattatatttcagtttaggaagaagaggggatttctgaaaaaaaaaagaatgaatctccttcctgcagcaataacaaagaaaatcattataaatctacaactacgtgaacaacctaacgttaatcctgctgaaacgccgctcagtcggtgcacctgagtcaaaacctcattccaaggtagggcaaatggccatttcatcttcccctttgccctttttagcttgatgggttttaacctcggtgcaccaaccgagtgcgagtagcattctagtcggaacaaacggaacaattagttccaggccgagtttagttacaaacttttcttcaaacttttaacttttctatcacatcaaaacttttctatacaaataatcttataactttttcgtcacatcgtttcaatttcaactaaacttttaagtttagcatgaactaaatacacctctagggagtcgaaccaaagtgatgcacttagttgtcggccgatgtggcatgctgctacgatgcacatcttgacattgacgtgtcatgaagacatgttcctccatcttgccacactctatactacctctatgacacgcgatgtccatgtatcaataatattactaggatgtatatgatgttacagatttgtgatatttttttcttttattactgtaacaaactaaaactggttgtaaatgttgtagcacttagatagtttagtcactaaatttacagtgctttaaaatttactctcgtttaataaatttgtatagtgtacatgcatgactagcacaatcgatcgttgagtcataccaacctgatgcaaaagcgctcaaactgtgcaggcgtcgatcctgggtgaggccatcaccgggaagggaatcctggcgcagctgaacctggagacggggatcccgatctacgaggcggagcccctcctcctcttcttcatcctcttcaccctcctcggcgccatcggcgcgctcggcgaccgcggcagcttcgtcgacgaccagccggtgacgggcctcgacaaggccgtcatcgccccgggcaagggcttccgctccgcgctgggcctcagcgagggcggcccgctgttcggcttcaccaaggccaacgagctgttcgtcggccggctcgcgcagctcggcatcgccttctccatcatcggcgagatcatcaccggcaagggcgccctcgcgcagctcaacatcgagaccggcgtccccatcaacgagatcgagccgctcgtcctcttcaacgtcgtcttcttcttcatcgccgccatcaaccccggcaccggcaagttcgtcagcgacgacgacgaagagtagaattacgaggatcgatcgatctaccgaggtttcgccgccattgttgtgcttgcgcatcattgagactttctttcaagtgtatatgtactgtgtacgtgtaggtgtaaagacgtaccttgtttaattagttgttaattaatgcccatggagaaaattaacgtgagatttagctacttgctgcggtaaatctccaattagtcttaaccaaagacatccaattagtcttaaccagagacacccatactttaggagttttggtggctattaatccattgagcatctatgcttgaaataaaattatctcttaaattattcatccgatttacaatccgattacactgttgtgttcgtaataattaaatctttataataagatctcacatgattatattttgatgaaaaatcacaaattacttttatgatatgtct

SEQ ID NO: 94 - DNA, 6775 bp, Oryza sativa
  source 1..6775: mol_type = unassigned DNA
cagcagacagaggtatgtcaatgtgttattggcctctacgttcggtagaaactagtaatgatgtactatattatgtgttctgaactcattataagatctctaccagcaaatgattctttgctattgtgctactatttgtatattgcgtttcatttatcatcatatgcagacaacctggatacaacatcgttgctcatattacaggtagaaaacgaacaaaattaaatggcattgtgtgccatcccgttttgttttaaagcttcgcctaaatcctaatgtattttagtgtctcgctgtgattgaattgtttccactaactgttgcatcatttgcactcttgtagtacttcatgtacactcttctctttaaaacgagactgaaatatacactgatgtatcatatccttttctcctgaatcttgttgactcaaagtagctcattctatcacgagtacttatcttcagcaagaacatcttttcatttttcttgttcctggatgtaatttggagctaaattgttttgagtactactaatatttaccaggaaccatttttgttactcctgaatctggtggcttgggggtaaattgcatacattagcaataaaaaaaagtaggtcccaccgggattcgaacccaggtcgccagattcaaagtctggagtgctaaccactacaccatggaaccggttgttgcatgtggtccgtcgatggtgatacttctgtcttttactgaagatcagtaacaaagggaagtatactagttgaaacccacatgttcatttgtgggcatccatctgctaggcacttcaattaaacaaaaacacgggaatatccaacgaatcctcttctccacattcccgtacttttttgtgctgaatcgaatttcctgacgccatataaaaaaacgaatttcctgacatatctcaggaaaaaaaaaaggtaatactcgagagtcgataacatcctcctagactcctacggcgtggcacacagttcactaaaatccaggcaacacaagtacacaacacgcagagtcacacatatgcttggagttggagcacttttccaggagtccggctcaggtgcagggaaccctgcacatagtaatatacaggccaaatcaaatcgcgacacctgtacacaaggtaggtccacccaatcaaccaagtaacaatgtaaacggaggtttaaccacgatcccacaaaatcaggtaacagaaccattcacccgcgaaatactttgacgtcgcgcgcgacgccgcacagagtttctttcctctttctctgttcttctgtactgctagatctctaaaattagattggtgattcttgatagagatcagtgtcctcgtgaagcaagacgcatggcggaagccccagtccacccttcatcttcaaagagtatgttaattgtccataaacttggcaagacccgattagtttgtctcaatctctgccataagttatctgctaattgttattaaattagattggtttcacctatatatggtgaataggattttatattgttccttgcatgttaaactagcatatacacaataatgatcacgttatacttattcttatatttagatcatgaaaaagaagacgtgggagacacgaatataagcagtactgatgatgcttgtgtgcaaaagttagagccagtaattggcatggaatttgatgatgaagatatagcatatgagttctacaatcggtatgctggagatgttggctttagcattagaaagttttggcatgataaatcttcaacaaatgtgattcgcacaaaaaaatttgtgtgctcaagggaggggttcaataagaggaatacttcagatgcatgccaacggaagcgagctgatacaagagttggttgtatggcagagatgaccattaagatcactcctactggaaaatatgctatagctagcttttctaacacgcataaccatgaactcataactcctagcaaggcccatttgttacgttctcagagaagaatgacagaagctcaaaaagctcaaattgacatcttgaatgattcaggtgttagacccaaagaaggccatgaggtgatgagtaggcaagcaggtgggcggcaaagtcttacatttaccagaaaagattataaaaattatctccgatcaaagcgcatgaaatctatccaagaaggggatacgggtgccattcttcagtacctacaggacaagcaaatggaaaacccttcattcttctatgcaatacaagtagatgaagatgagatgatgactaatatattttgggcagatgcaagatcagtattggattttgattatttgggtgatgtcatatgctttgacacaacatacaggacaaataattatggtaggccatttgctctttttgttggtgtaaaccatcacaagcaaacagttgtttttggagcagcgttactgtatgatgaaacaacatcaacatttgaatggttatttgaaactttcaaaagagccatgtcaggcaaagaaccaaggacaatattaactgatcaatgtgcagccatcattaatgctattgggactgtctttcccaactcaactcatcgtctctgcgtttggcatatgtaccaaaatgctgcggtacatttgagtcacattttccaaggttcaaaaacatttaagaatgattttggcaagtgtgtttttgattttgaagaagttgattagttcatatcagcatggaacaagatgatagaggagtacaatttgaatgataaccaatggttacatcatttatttgagatcaaggagaagtgggctttagtttatggtcggcaaacattctgtgcagatatgaaatccacacagagaagtgagagtctcaatgccctgctgaaatatatgcaacatatggattggttcttgaaattttcttttatttatatagagcaaacactcaggcacaaaattgggctttttttacatctcgatctgcagttcaatgtcattaatatttccatttgcaatctaaacaatatatctatgcttatggtgtgtatacattatagtacaagaaaaaattattaagcagttagacattaacctgtgacaggcttggtcgatccagagcaagcccaacctaatccatcaggactgcatgacctctccctgctctaagtctccatggcgaccacggctccttccgccggccactctcccacggtgagcgatgcgacctcctcaggacgccccacggcaagtgcgcaagcctgtagtggcacccggcaagcaccgccgccccaccccggcggcctgcctcgagaccgtcgtcctcttcgatggcccatggtgagcaccacttcccagccaacggcaggtgtgcaggcacatttgcagcagctggcaatggcgtggtaagcattgacctcgctcccgacagcttgccttgagagtatggcgccatgtgcaagatcttttggatgtgctttcactgctttgtgatgcagttcaggagtggttgaggaaagagaaggctggagaggaaaggggatttttttatgggagtgagaaggggattaccaacaggaaagagaaatccagataacagaagttaacgtgatgccttgtgccgtttagtcctcgttaaacctccgttgcatgcgtaattaagtagggccaccctggacaggtgtcgcaatttgatttggcctgtatattattatgtgcagggttccctgcacctgagccggactccttttccagtgacgcgtgcagtttaccaaccacctgctcttctcggccagttcgctgcggacctccaaaacccaagcggagatgatcgcgcaccgtgtcagcgcgtcgcggccatagaagggattcggggagacgtggccgcccgcgcgcccgtcgcgcaccctatcctcgccgcgcgcccgccggccaatcgccacccctgaatccacagccacgccacgcggcccatgcctacgcctcccacccgccacaggatctccctctcacgcgcgcgcgatctcataaaaacctcgccacctcgatcggtgaggtagcacgaggcggcctgagctgaacaccgcatcagcagcagcaagcagctaatcgcgcacgtacgcacaccgcgacccggggcgagatggcgcagtcgatgctggtgtcgggagccaatggcacggtggccgccgcgagcaccagcaggctgcagcccgtgcggccgacgccgttctcgcggctcgtgctgtcgcagccgtcgtcgtcgctggggcgcgcggtgtccgtcaagacggtggcgctgttcggcaggtccaagaccaaggccgcgcccgcaagaaaggttggaatttggatggatgaatgcaagctaaactaagctaagctaagctcgcgtttgttttagtcttatgtttgatggtgctaactattttgtaacgtgtcaacgtgtgtatgtaggctgagccgaagccaaagttcaagaccgaggacggcatcttcggcacgtccggtgggatcggtttcaccaaggagaacgagctgttcgtcgggcgtgtcgccatgctcgggtttgccgtaagtagacctttcttggccttctagttatttggtatgcatcatttcttgccatcttggtacgtacctgtgcgttcgggtttgggttagctagctagcaagagcacaagcttaaacgtcaaaagttttgtggttaacggaagcactttacccgggagcgtcatcctttattctataaagggtcacatgttgcataggtagatttgcttccaactacttttttttaaaaaaaaattatagtttttgtcatatattttactttgaattttctgtgcacataaaaatgctatatttctccttagcaaatattgctacttttattttatttttcagtccgagttttgacagcgacattgtttaatctaggccagttagcaactcacatgtataccacacacataactaatgaattatctggatttagtaatataggtattgtaggttttaaaaaattcgtctttcgagcagatatattgtttaatgagtaaattttagagaactataaatttagtgactaaattatcagcttgttgtaacgttagtgacatattatatttcagtttaggaagaagaggggatttctgaaaaaaaaagaatgaatctccttcctgcagcaataacaaagaaaatcattataaatctacaactacgtgaacaacctaacgttaatcctgctgaaacgccgctcagtcggtgcacctgagtcaaaacctcattccaaggtagggcaaatggccatttcatcttcccctttgccctttttagcttgatgggttttaacctcggtgcaccaaccgagtgcgagtagcattctagtcggaacaaacggaacaattagtttcaggccgattttagttacaaactttttcttcaaacttttaacttttctatcacatcaaaactttcctatacaaataatcttacaacttttccgtcacatcgttccaatttcaactataaacttttaagtttagcgtgaactaaacacacccctagtggagtcgaaccaaagtgatgcacttagttgtcggccgatgtggcatgctgctacgatgcacatcttgacattgacgtgtcatgaagacatgttcctccatcttgccacactatatactacctctatgacacgcgatgtccatgtatcaataatattactaggatgtatatgatgttacagatttgtgatattttttttcttttattactgtaacaaactaaaactggttgtaaatgttgtagcacttagatagtttagtcactaaatttacagtgctttaaaatttactctcgtttaataatttgtatagtgtacatgcatgactagcacaatcgatcgttgagtcataccaacctgatgcaaaagcgctcaaactgtgcaggcgtcgatcctgggtgaggccatcaccgggaagggaatcctggcgcagctgaacctggagacggggatcccgatctacgaggcggagcccctcctcctcttcttcatcctcttcaccctcctcggcgccatcggcgcgctcggcgaccgcggcagcttcgtcgacgaccagccggtgacgggcctcgacaaggccgtcatcgccccgggcaagggcttccgctccgcgctgggcctcagcgagggcggcccgctgttcggcttcaccaaggccaacgagctgttcgtcggccggctcgcgcagctcggcatcgccttctccatcatcggcgagatcatcaccggcaagggcgccctcgcgcagctcaacatcgagaccggcgtccccatcaacgagatcgagccgctcgtcctcttcaacgtcgtcttcttcttcatcgccgccatcaaccccggcaccggcaagttcgtcagcgacgacgacgaagagtagaattacgaggatcgatcgatctaccgaggtttcgccgccattgttgtgcttgcgcatcattgagactttctttcaagtgtatatgtactgtgtacgtgtaggtgtaaagacgtaccttgtttaattagttgttaattaatgcccatggagaaaattaacgtgagatttagctacttgctgcggtaaatctaattggagatgcctctccaattagtcttaaccagagacatccaattagtcttaaccagagacacccatactttaggagttttggttgctattaatccattgagcatctatgcttgaaataaaattatctcttaaattactcatccgatttac

SEQ ID NO: 95 - DNA, 4064 bp, Oryza sativa
  source 1..4064: mol_type = unassigned DNA
cacgtcctccacggagggaaggcggcgcaccgcgtccgccgccgccgccgccgccgaagcaacctcctcctcccctcgccgcgacagcgcgagccgctccaaccggctgacccaaaaacgaaaacaaaacgaaaaaaaaactgagatgaacgaacacctgatccgcgcgggcgaatcggaggaggacggcggagggtttagccacctaccttcgaatggccgccgcgtcgagcggcgcgtcggcggcgtcctcggctagggtttccgccattagcagcagcagcaatggcaatcccctcccctctcccgcccaagaaaaatttcccccgacttcgaaaaaaatgggtggcgattttgagaaatcccccctccaaaaactccgaactgcgatctagtccgcggccacgtcgggccaaacgggcggcccggcccagcaggcacggctcctgttgcgggccgtgccgtgctggcccacgtgctaaggcctcggaccaagcacggcccagtaaggccacgggccgtgccgtgccggcacgggcccaaatatagcacgtgggccggtgtaggcccattagcacgtttggcccacaaaattgaatatgaggccgaaaaataaagaaaaaatgtgaaaagaagatgaaaaaggaaaatttgtgaaaatattataaaaaatgacgaaaattagtgggaaatagaaaacaacatatggaaaaatcgggggaaaacgagagaaatgagagaaaataaacaaaaatatatgaaaagaaaagggaaaacaggaaaaatttggtgggccaacgggccgtgccgtgccgtgccggcccatggtctcatacgtcggcccaagcacggcacggcccacaagcgggcgtgcctgggccggctacagtggttaccgaggccttggcccggcccatttggccagcactcgccccttgcggcatcgcctcctccccgccacacaaacaagaaggcgagccaagccaagccaagcccctccgcggcgcgcgcgttatccatctccccctcgccgccgccctatcgccgctgctcgagctctcgatcccctcctcctcgctccgccggccgccgcggccgcaacccaggtcagaggaactcaggattccttgtcagttcttgcccgggctcgcatttttccttgctgggtcttgaatctttctttctcttcttgttttgtgtttcttggtcagatgatgccgcggcagtgcgggaatcgcgcgttgctagcggagggatcgagcacggtggtggtcgtccatggccgcaagaccagaggtggtatcagcactgtcacctccagcaggaggaggagccatggcggtgtcaggtaccaccggtgctgccccccgagggctcacctatggaggaagggcgatcatcttcctctccatcatgccaagatctctgctcgttgttccgaggtgattgaccgagtcctcattttcttgtgattgcacactcccaattttgctttcttattgacaaggaaaaaaaggggattgggttttgggtgatttgtttgatagatcaaggcccacacggtgctgcaaggttcagatgctctaagcagcatcagggagtggagcaggtcgcatttggtcaccatgacagggctggtggcgtgtgctgttcttgtagtcccttctgccgatgctgtggatgctctcaagacatgcacttgccttctcaaggagtgcaggtgagtgggattccctttttttgcatttgttccaagcatgattccaaaccgtgtgcattcgccttgtcgaacatcgatactgtaatttttaggaaacattactttgttttgcttaactgaagtccagcaacaccacatgccattgttcaaaacaatgatacaccagcatctactgaatctatgcaattgtcattttgtcaatacagatacagttgatcctgtttaaggcagtacatttttacaatgtttaatcagtcatacaagtgagttctgcggatcttgtgtgttcatatgtttcttataccaatgtaatggaatcaatttcagaatagagctggccaaatgcatagcaaacccatcctgtgcagcgaatgtagcatgcttaaatacatgcaacaatcgccctgacgagactgaatgccaggtggttccatttcccagaaaccaatcagttcagatcatttcctcagaagaataacctgtgctgtatcttgtcatattgtagatcaaatgtggagatctgtttgagaacactgtggtcgatgagttcaacgagtgtgctgtttcgcgcaagaaatgcgtcccacaaaagtccgacgttggcgagttcccagtccctgatccatccgcccttgtcaagaacttcaacatggctgatttcaacggcaagtggtatatttcaagtggcctcaatcccactttcgacacattcgattgccaacttcacgagttccgtgtcgagggagacaaacttatagcgaacttgacatggagaattcgcacccccgactctggcttcttcaccagaacagccatacagcggtttgtgcaggacccagcacaacccgcgatcctctataaccatgacaacgagttcctgcactatcaagatgactggtatgtcccttagatagcaccttctgacgctgtacttcagatttattactaagattttctgaaatgtaggtacattatctcatccaaagtagagaacaaggaagatgactacattttcgtgtactaccgcggcagaaatgatgcgtgggatggatatggaggtgctgttctgtacaccagaagcaaagttgtgcctgaatcaatagtacctgagctagaaagggcggcaaaaagcgtaggtcgtgacttctccacattcatcagaaccgacaacacctgcggtcctgagccacctcttgtggagagaatagagaagaccgtcgagcaaggagagaagaccatcatcagggaagtgcaggagatcgagggcgagatcgagggagaggtgaaggagctggaggaggaggaggtcacattgttcaagaggttgacagatggcctcatggaggtgaaacaggacttgatgaacttctttcaggggctaagcaaggaggagatggagcttttggatcagatgaacatggaagcaactgaggttgagaaggtcttcagccgcgccctgccaataaggaaactaaggtagcttctagttgccactccagcaagtacaaggcaacaagtaatgctcaaaatatattctattcaccaaggcaataccaatagtaacagattattctacagagaacaaagaatttttgaatctgctaatgcatccccattattacaatacatttccagcttagagatatgtaaagtatcagtaactatgtaacttccattaaaaaaaggtatatagtgaactttgtacaactgttccaaatatgatatggattatgtccttttgatgacgatagtttgaatgctgttaattttcttcggtactttatggcaatttaggttatgtcaaatgcaaataatattgaattgtagtttcataatttcatgaacctttcttttactaagaggctctctagaaactttcctattcatcgttaacaatcactgttcagtgggagcttaaaaggagcttgtattttattagtatgaacttctcaagcagcagcctgaaatggttagatggagggtgagagcatatgagatttaactggttgttttcataatcttagcttactctttatgttcttgattcaaacaaggaatcatatggtagctgacaaaatacccctataatcatccaggaagacacagaagcaattctggaaacattttattatcagcaaggaaaatttctcactccatgcttcccagagccaaacggcaccctatatatgtttcgaataatcatagaaactagatgactggaaaattcaattacagatacatactcacaccgacaacaataaacgtggaaaacaatacatgaacagagttcgatgaaaattgaaaattcacaatgtagcaatctgatcaacaagtagctaaggaa

SEQ ID NO: 96 - DNA, 10395 bp, Oryza sativa
  source 1..10395: mol_type = unassigned DNA
tcaatataccactgtatcatatctctctattgtgctagtattaatgaagctgtcgataagtatttgacaaccgaaacaaataaactttggacatatgaatatgagaatttatcctccctcctcacatacattatcgttcattcctcaactttcttaactcatctcatttattttcacgctcacgtttttcaactgctggttttttagaagttgctttataaattcatattaatccatttttatgtgcagcgagttttcaagctcacctctgaacacaaccttagtagatgttatcggctactgagaattgactgctgtgaatgggttcgcttgtagacacgtcacctcgcactggtaggtactggctggttttggtgagtcggttaatcgtgcattcctttggcgtcttacagttttgtcaaaagataaaaattaagaaagtcacgtctcataaaaagtatctcggagtgtcactctccaacaggcacacactgcttcaggagaaccggatgaagccatacgccaactgagaaaagatctccaaactcacctatgaaagagcaatttgccacgtctccacgcgtgcttctctagttcgttttctgatcttctgtggatattattactgcatgacgttttgcttgctatggaatgcaccttttcgagccaagccaacccaacccatgataaatacaaagatataattaggaaataagtccgaacttatttgtccacgcctatcaaaatctataaagttgtgtaaacaacaagaaactatgtaaaatgtaattgtttagttaatgacataaggataaaactataatgttgtggtatataaaaccgaataaaaaattactgaatacaaaacgcttttgataacgtgggaaaacacatttacaaaacacctaattctaccaaacctttacaaaccgcttgggctatactcctcccatagaatttaatggtgtgaatggacatgcaagttaatatgtcatccgcacctccgtgctagtaggcatatggttttcgagcggtctacttcagttaatcatgcatccctaagttagtaccttgcaaattttatcaaaagattagaaatgaagttagtacgtataaaatatctagtacgagtttatacgagataatagatagaaaacaattaaccttataggttgaaataggattggaaacttatcttcataaatagttgtaattttttcttacgctttttttagaggtatagatatttgtgctaattaagaggtaatccatcctttccaaattgatcatcatataagtttatgcaccaagaccaagaacaatttaaatcacatcaatcaagacaccatgcacatattctcccacaatgcatgcatcgattaaaaccctataccaattacaccctagcatgcacacattcttccatattgtattaattgtattccaatgaaatccatatccatgcagttatatgatgattagaaattttggattttattacatgatgatcaatttgggaaggagggagtatatacctttagggtggagatatattcttcggactaacactatgagtatatatacctctctatacctcataacaacttaaagttttgtagtgctaatcataaacttatatttttcaatcagttaaactagtttaatttttttacctcatatattatgagaatgtatatacttttgcaatggaagaaaacctcaaaaaacaattccctttagaattaacggtgagaaggtcccagttcaaacgttggacatgtaatctcaatatatatgagtatttagttctcaattctgaataaaatttacatttgaaacacgaccaaagagccaataattagcttgtctaggcagttggccaagcttaacaccgccgcaataaaattaccatgatactaatttttaagacgaaaaccacgtaccataatgtaatgttataacaaagaaaaatctactccattgtaagtgatacccccatgaactgtgttccacaatattttgatctccaccataagtctttcaattctctaattatttgatatatgcctaataaaccatgaaaaaaataaactaagcaaccaagagtaatttattagcacaaacattaacagtgttcatagcggattaaaagccaatgctaaaaatattacaaactttaaaataaactaaaaattatgtttcaaaatttaaattttggatccagttgacgacgagagactagcagtttactgctttgaatactctggtaaaaaccggtgcaccgctcttcttctacaaatggacaactaggttttcggctaccgttctacacggccacacatggttagccacccagcggtccgcgcacagaagatggtgagaacctggcaagataaaatcctgagccagcgtcatccgaccttccaaatggaatcaggcgattggctgcggttcctcctccctccaccaccacaccaccaccggtcgcctactcgccttcgccttccctcctctcctcccttccacgaccgcagcccaaaacttctagaagcaaccgccgcgagcttaactgctagtactactaacacctcacctacctccctcccccgccattgctccgtctccgctacacgcgaagagcttcacctgactctcgcttgacgtgctagttagtagtagctctcgatcgaggcgcggtcgtccgtcgagggcgcgtggccatggcgctcctctccgcgaccgcccccgccaagacgcgcttctcgctcttctcccacgaggaggcgcagcacccgcacccgcacgcgctctcggcgtgctgcggcggcggcgccagcggcaagaggcagcgggcgcgggccagggtggcggcggcaatgcggccggcggacgcggccgcctccgtcgcgcaggcggcgtcgccgggcggcggcggcgagggcacgcggaggccgcgggtgctcgtggccggcggcggcatcggggggctggtgctggcgctggcggcgaggcggaaggggtacgaggtgacggtgttcgagcgcgacatgagcgcggtgcgcggcgaggggcagtaccgcgggccgatacagatccagagcaacgcgctcgcggcgctggaggccatcgacatgtccgtcgccgaggaggtcatgcgcgaaggctgcgtcaccggcgaccgcatcaacggcctcgtcgacggcatctccggctcctggtacgccactcccctccccttcccttctcttttcccttttcggatgaaaccatccggttgcatcagactccagtgagcaatgtcttcgtaagttcgtactcgtacagtcgtactgcaccacagtgtaactaaaaacattcatttcagtcacgccatcgtgtgtttaattaacgattctgaccataattaagggcttaataataatcacctcgcggctgcaaccctagcagtcagactatgagtcctgaagggtgtgtttagttctttgggtgtaaaatttttaaagtatacagatacattcgaagtattaaacgtagactaataacaaaacaaattacagattatatctataaactgtgagacgaatttattaagcctaattaatccgtcattagcaaatgtttactgtatcatcacatagtcaaatcatgacgtaattaggctaaaaaattcatctcgcaatttacatgtaaactgtgcaattgattttttccccacatttaatgtcttatacatatgtccaaacatttgatgtgatgttttggccaaaattttttggatctaaacaaggccatgtagttataattttgatccacgttttaataccaaaattttactctgaatcatcctctaactaatattttcactttagataagattatcgttgttgtactttggatcatctaaatcatttctctggctgcatctaccatctaatcttgcaaagaatagcctatatgtatgtcaggaatattatcggtttacatgtaccgatgtagttaaaattatttatacgctcgagagaagtttttgaggtggttcaaactaaaataaagatagctaaagtgaaaaaatcgtttaaaagacactacgttcaaagtgatttttttaatacgggtgaccgtaaacgtattttactcttttgtttactcgagtctccagcatgttacttgtgcagttgctaggcgcgccgctgatctcacgaacacacgtggtgcgacgccgcttaggccaggcgacccacgttggagccgccccaccagccacggcaaattgaaaagaaaaacacctggttagagcaagtttaacagtatagcccactactatctccaaatcatctatagccaatgtaatagctaattcatataatagggcttactatactatcaatagctggtcccatttgtcataaatgtcttggagcctgtgctgcagctctgtagtcaactactcttctctctcttcttttatctctttaaaatatgtttatagctggtttagagcaggtacaatagcaggctataagctaaactataaatatattttaaagagataaaggaggagagagaagagtagcgggctatagatatgtagccagctgcagtacgaactctaagatgtaatgtgggtatgataggtaggaccgggtattaatagtatagtaagcaattatatgaattgactattagattagatatagatgaattgaagctagtagtgggctatactagctctcatcagtcagctggtcggcgtcggcgtcgtcgtcgctgctttccccttctcctggccccgccggcgagcaacccgcgcccgccacgtacgcgcgtcccacttccccccaccccacacgctgctccactacgcgccgaaagggtgaaaccacccatccggcccttccattccgcggcgggtgcctccaattcgtcgagcacgagcgagtgagcgacatggcttctaacacaccaattcgtcgaacacttccgatcgtcttctctgtctccgtgtgagtattgacctcaggcgccgcgacgtctgcaagagtagtcagattcaggagatgctcgttgggtagagcggatcggagagtcactgtcacagtagcgcttctgctcttgcttctcggatcccaccacccaagaagagctttgctttgctcccgttccactgccagtgccagccatggcgccactcccaagctccagctcaacgcgggccacctctctcgtggcgatcccgggcgcctcctgcccccatggccaggcgcaacgcctgctcgccttccgtcccccacgacgcgggtcacagtggcgcgggctttgcgtgtcccgggggcgccacggggcgaccgtcgccatggcaatgccggcggcgcaggcggcggggcgcagggcgcgcgtcctggtggccggcggcggcatcggcggcctggtcttcgcgctcgcggccaagcggaaggggttcgaggtggtggtgctggagagggacatgagcgccgtgcgcggggaagggaagtaccggggcccgatccagctgcagagcaacgcgctcgcggtgctcgaggccgtggacgccggcgccgccgaccaggtcatggacgccggctgcatcaccggcaaccgcgtcaacggcatcgtcgacggcgtctccggctcctggtaaggagtcaagtcgtgccagcgttagaagcgaattgcaacacccaccaaggattggttgcgtctatcaagcctaaaattatggggtttttttttctcctttctagaagtactaagtgtaagaataatttgctggtttacactattggccgtcctgtttgtacaagttttgcttacctattgctacaacctacatgagaatcattaatcacagacctatagtttcaattacagtacaatactgtacaaggaaatgcaatttgttgcttaatcattagtgctaacttgacatataaggcaattattctttgtgcttactctgctacatttgtttgcatttgcaattctgaatgttgttgtctgcttgttggttagctttaagatgggaactcaactgctgaaggcatccatttgactgtgtcaaattggtattcaggtacatcaagtttgatacatttactcctgcagctgagcgaggtctcccagttacaagggttattagccgaatgacgctgcagcagattcttgctcgtgcggttggtgatgatgctatactgaacgatagccatgttgttgatttcatagatgatggcaacaaggtgaatgatcagtttgtcattgtggtcaattggccatatcctcagtataaaatgtgctcaaaatgttatggaaaattcttatgttatggcttttggacaagtacaggtaactgcaattttggaggatggccggaaatttgaaggtgaccttttggttggtgctgatggaatatggtcaaaggtaatgtaatcactcaaatcttttggtaacaattgacaaacataatcaacagagacgtgatttgagaatttaattattggacaggtgaggaaggtgcttttcgggcaatcagaagcaacttattcagaatatacttgctacactggcattgcagactttgtgcctcctgacattgacacagttgggtatgtagtgcggattcagatgactgctgtatttttttcctctgttaggaaatactaatatgttcctcattttcttctgttgaaggtaccgtgtatttcttggtcacaaacaatatttcgtctcctcagatgtcggtgctggaaaaatgcagtggtatgcatttcataaggaacctgctggtggcactgatcctgaaaatggtgagttcctagagtgaaacttttgagctcacaagcatatgcgggccctgtctgtgttcttattgtccactaatgctttttattggcaggtaaaaataaaagattgctcgagatatttaatggttggtgcgataacgtcgttgatctgataaatgcaactgatgaggaagcaattcttcgccgggatatatatgaccgaccacctacttttaactggggaaaaggtcgtgtcactttgctaggtgactctgtacatgctatgcagccaaatctgggtcaaggtggctgcatggctattgaggtacacaattaacttttcatgtctctgatgacactaccagcatggtggatgcatttcttgccctggtattgaaactcagcatatatacttatgttactgtggcaattttttcccacttgatttcacaggatggttaccagctggctgtagaacttgagaagtcctggcaggagagtgcgaagtctggaactcctatggatatagtttcctccttgaggaggtaactgttttttatgttttatcatactagttttacctagatagaatgtcttctggcatcgactagactttatgtcaatacggatcaaaattattcttcaaggccatcttattgtgtatactcttaattctttaggtgtgatcctcagtcatttgattgatgatcaagacattattgtgcttgtgtggctgcagatatgagaaggagagaatactacgtgtttctgttatacatgggttggcaagaatggcagcaatcatggctaccacttatagaccatacttgggtgtgggtctgggaccattgtcggtatgtcaagaacttgttctgctttgtacttctaatatctaaatgtacttatgtggtttatgcccgtcagccagttttaacctgcttattatgtctgttgctgattattttattttatttctgaaacaagtttctgatttagattatcatcataagaaaatcattgaagaatcgcagactaaaattatttggtcttaatatggatatctgtccaatgcagtttttaacgaagttgaggataccacatcctggaagagttggtgggagattcttcatcaagtatggaatgcctttgatgttgagctgggttctaggaggaaacaggtgattattgagctataacttcgattttattttgagctcacatgtagatacataatctgaaaatactcactagattggcatggttgttattactcaccatatgttaatgtcaccaatgaacagcacgaagttagaaggaagaccgttaagctgtaggctttctgacaaggtatatctctattctgtatgatttatcaatgtggggcagtgcaatattttccttttctgacttttacaatcatgctgaaggcaaacgaccagcttcgtcgatggtttgaggatgacgatgcattggaacaagccatgggtggagagtacggtcttacttgtcttcagaattttgatttcattttatgtcaaatatgctctgtataatccttcttaattccagtgcctttgagaaaaggtactcattagaacaatgctacttgcaggtggtacctcctccccacaagttctggagactcgcaacccattcgattaatcagggatgaaaaaaagtcactctccattgggtaggtctaaataataaatctgttattttttggtaacactttcttcaaggattgtagaataaaacaaaattgttcttttgcagaagccggtcagatcccagcaattcgactgcttccctggcattgcccttgccacaggttagataagcaaaatgatggcatcaaatatttgttggaacttctttcgaactataaatgcattgcaacggttcttaggtggagacattcaacagctgaattttgttttacttattactacaacctaattttctcttgcctaaaaaaatcttcagttttatttctgtcaataaaaaactaatgcttctccaatttgttcaacttcattgcagatatcagaaaaccatgctactatcacatgcaagaataaggccttttatgtgactgataatggaagtgaacatggtacatggattaccgagtaagttctgaaaaccgcatgattctttgctcttgccctgaattcgaaatggatgcatcttactaattcagtcccatgtggactctccagcaacgaaggtagacgctatcgcgtacctccgaacttccctgtccgtttccatccctcggatgccattgagtttggttcagataaaaaggtacccaaaccttactgacaccgtattgtaccattgtctcctctcattttgttcagaagaaccatttgcgttgagttgctgcaaaaattctgagttctgatcaatttctacgaacttgcaggccgtgttccgtgtgaaggtgctgagcacgctcccatatgaatctgcaagaggagggccgcaaatcctgcaggcagcatgaacgagagacttcagtcagccatggctgctgtcatcactcatcagctactgtacagcatccagtaaaacacagtgaattttgcactaaaatgcaggttccacttccagacaggattttgaatagctttctctgacggattgaaattgcagttctccatacagaaactgaggtgtacaatataaaaaaaaagaaactgaggtgtacaatagccaatagggacaggcaattttagctgttgcctgctgtatatacattgttgtgccattacagcaacaaggtggaatgtaattacggcatgaacacaatttaccaaatggaaaaaaatgtagctaaaaaccgctttgtttggtaactagttgtacttttccgaactttcttccaagcggaggagccttaccttgccacgatgcgatctgatttctgaactagaacgtcagccacaatgcgccacatgcaccaccgcagagcgccgccgccaccgccgccgccgccgcagtgttcgccgccatcgccgtcgggaaggcgaggtcatgggagtgtgagatttgggccgctagttcggcgtaagagcccttcccagccgttggatcaccattgctcggtccagatttacgccaggttaggcttgtagaatctgcgaggcgtctcctgcctggcgctagccgctaggctccccactccccgcgagcttcagccatgtccgcccgagccgccgccggcggcggtgacggcgatggcgggggccgccgcaagacccttgccaaggtgtccctctcctcgctctccgccgccgcggcggaggcggccacgttccccatcgacgccgtcaagacgcgcctcgagctccaccgcggtacagggggcagcggcgggggaggcgggggcgtcatgcgcgtcgctggcgagctcgtacgcgacgggggaatctaccggggcctctcccccgccgtactccggcacctcttctacaccccgctccgcatcgtggggtacgagcacctccggtccaccttcgccagcggtggacgagacgcgggcctcctcgagaaggcgcttgctgggggtgtctccggcgttgtcgcacag

SEQ ID NO: 97 - DNA, 4184 bp, Vigna unguiculata
  source 1..4184: mol_type = unassigned DNA
taaccacttcaaacataatttaataattagttctgtaaggtatttttcattttatcataccttaattatacatttgatttcctttgtgcgatttctttcgatagtctctcaacttatttctaaaacacacatttgttagtcttttaatatttttatttattgtttattttcatcatgtagaataatatttcgatatattctatctaattatttgttattttataactcactattaatcatactgtaattttttccactttaattgtataaataacttttatttactacaatataaaaatttaatgtaattgctatgaatatttttttgtcactatccaacatatattgaagttcaaaagatacaaaatctatgtcaaaattttattattatgtttattatttgttctttgtgtcattttgatcaagtgatgtattataacttttattagtgtataaaaaagagattcattataatttaaaaaattgaagtaaacaaacatttaaaatatattttaatttgaatatttgttttccttttattattatgtttattatttattctttgtgtcattttgatcaagtgatatattataacttttattagtgtataaaaaagatattcattagaatttaaaaaattgaagtaaacaaacatttaaaatatattttaatttaaatatttgttttccttttatattttttgaaatattttttaattttatcaactaagttcatcaatgttgtagtttgcaaatttaacaaatgttttggttcacatgaaattttatttggtattctaatataaaatgtaaacaattataatttattttaaggtatttggcatcgaagaaaatcctcctaatattgaatatttcataatattatgttttctttaccgtaattttttttcttttataaaaattaatattgaagtagttagaacacgggtacgggtatgggtacgagattatacccgttacccggtggggatggggatgggaaaaaagtttgatacccgttgggtttgggtatggggatggggatgaattttttatgcggggatgggtatgggatagcgaaacccgtccccgccccgccccgttgccatccctaatgaagacaaaagacaaaatgtgcagcttataaaaaattaggacaaaatgttttttactaatatatatatatatatatatatatatatatatatatatatatatatatatataagggtatttttgtgaattaaagtttagcgggtacgggtatccacgggtacggatactatgatacctgtacccgcctcgttaacatgcgggtatcaaaaatacccgtacccacgaatagcaggtatccatttttaatatcaatttcttacccattgcgggttttatctgccacggatatgaatatttttgacatccctaatcaaggtagagcagatctcactttcaaaattatttttggatgttagacttacgatgaaagatgcttttgcatatcaatatcagtgaaaattttgtacttccaaatattaattgaacatatatatttatataatatcgaacatcgatatctataatatatatatatatatatatatatatatatggaagtcgactttcgatatatatttatatatatttttgacatttgaaagtctgtcattttcaccggatgttaacagtcataggtattctttattgaaagtcgatatttggaagtaatttggaaatgaaatttgagttagtctaattaaataatcaaagagtatttttggaatttaatttttttttttggagttgtagaagaaattttgaaggtgtaggaagaaatctccatatttgtttgtgccacgtgggtggtttcactaactttatcatttttgcactcattctttgtttgttaggaggatagaaagggactgagaagaagaagaaggaaagatgaaaaaagccaggaaaaagggaaataaacaaaattatctgtattaaaaataaatgaataataataatgtaaaaatttacctaaaaacaatgtaaaacatgttataaaatcttttacaaatcgtacatcatacaattaccaaatctatttagttatataaaacaacaaatttcttttttgtttactaagagatgtttccaataagtaaagagaggaaaaaatctccactaaaaatagtttacaaagtatggtgaagttatgaatgaaggttgtggatacattttcacgtgcgttaattattaaaccgttattttagagaagtatttttttgatggtaagataacaaaaactgtgtactaggaatatccagcggtggttgttaacgtgtcaaaagaggatttgtggagttgagatttccttgtccaatcctcatagctacaactgactaacttctttcttcagattctggaccgcaataaaacccaacttagaatttggaagaatctagacacattgcttttcaaaaacttcaacttcaattattttgtacgttaataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgttggagacgtggtcactcttgttgcctgtcatcaactattacaccacatccacacccttgttaaattatagatggtgttcctcttctatcatcattttggtaaaccctaccttaacatattcaacactaccattcccaacattctgaaccaaaccaccgaggtaaggtaacacttcgtttcatactccaccaatggcgcaaaccatgttgctcatgtccagtgtctctagcagccactccctgggtttgaagaaggatcttttcctccaattgaggcctaaattctctcaactctccttcaaccctcttccatcttcaactcctttcccttcttcacctcgtacattcacaactctcgccctcttcaaatcaaagaccaaggcccctcctgccaagaccaaggtactcattttccattcttttatgtaaatgaaacctgtgtctcagaaaattacgagatttacgttgtgtattcattaattgaggttgtaaagccaaagcaaaaggttgaagatggtatctttggtacttctggagggtttggttttactaagcagaacgagctctttgtgggtcgtgttgccatgcttggttttgcggttagtaaaataaatcttaccttgtaatttaataaacagtggcatgacgtcatattttattgtgagtcaggaagatttataaaaacatttgtgtaggcatcattgttgggtgaaggagtaactgggaaagggattcttgcacaattgaatctggaaactggaattcccatttatgaagcagagccacttcttctgtttttcatccttttcacgctgctgggagctatcggaggtttaggtgaccgtgggaaatttgttgacgatgaggaacccaacaccggaggtgttattcccggaggcaaaggcttcagagaagcacttggtctcggttcaggtacctcactttcgaaacaaacaatgttgttcaatttcggagttcttatagtaacatttatagaagagtaacatttgttgggttatttttcaggtcctttgtttggattcacgaaagctaacgagctatttgtgggaagattggctcaattgggattcgttttctctctgattggagaaattgttactgggaagggagcactcgcccagctcaacattgagaccggtgtaccgatcaacgaaatcgaaccccttgtcttgttcaacgttcttttcttcttcgttgcagctttgaatcctggaactggcaaattcgttacagatgagggggatgatgaatagagaatagctctttttctttctttatctctgtttaaacaatacttccttccttatctatcatcaatgcattcagttcacaacaacaacaaaaaaaaaatcatcacacttttctgatctgttttaccctattatatcctacttcctcatacacacgttacacgtgtcaccgaacaagcttaccggtgtgggaattaaccattgcttggtgtcggcgcaacacgtaaactaagaaaatataaataatttgttttgtttgtttgttt

SEQ ID NO: 98 - DNA, 4955 bp, Zea mays
  source 1..4955: mol_type = unassigned DNA
ccccatgaaggaccttggtcccctccaacacttccttggggtcaatgttacttgcaccggtggcgggatgcttctatctcagcggcagtacaccctcgaaattttggagcgcgctgggatgactgagtgcaagccatgcaataccctagtggacactcagggtgggggggggagtaactattttgcctgcccacttaggtttcgaccactggtttgtgcagttgcatatttgttttgactcaaatgcttttactgagattgaattggtggcatttatgaaaccatcttagagcccttttttgtcaacagattcagttgatgtgttcattcatgtatataaataagggtcccccagaataagtagtcaatctcatcttccactgtgagcagttgatgcttttgtttgataattattctattagagttccccctagttgtagtgatttatctgaattaccaagttgagtcacagcagaaaactgacaaagtttatggttgcagctgcactccatatttttacatgtaaggaaagtcaggcgaatggttggatttcgggattttgacagaacagtggtgaagctggaatgggtactcaactggactacctttgtaacagcaagggtgatatgccatatcttgattacttacaagctcatctctgatgcccacaaatttgggaagggcattgagcttccgctagctctacttggtatggcaggaatgaatctgcttaacatatttcttgggctcgatctacttaaagcattcacacgagaaagaaataagcagaaccatcaggattgaatgcacctttcttgatgacggtgtactctaaattttgagaaggctgtagttccgttcttgtagggacatagaggtatgtttaagtcttaattaatcagcaactacactggaggctatggattctttatgtatcaacacgtgctaatgctaagatctctacaagaaagggttgttaatgtgtaatcatgtaggtataacattattgttcattaggagaattaagagagtgcaggcgggtaactggatgcaggatttacatacattttgcaatcaggaatcggaaagaagagaagcgtaccatgtatcaaaccccttggtcccaactctcatacctcatctaaagtaacacctaagcaacttgtagattggcactgtgactttgaggctacaatgctcttgtttattaatatttataatatttgacgatatcttgctgcgaagcaaatattgtgtttttttatgtatatgcatgggctccaccaccttcactcggctatcgatgatttcttgctgcgaaggaaatagttttcttgccttaaacctggattggctcattttgacgtcttttggccttttgggtcgacccacagcggaaaggaaacttttatggattctgaattgtatatatatgttatttcttttcgcaacagggaaaaaggtgcattctgaattagacccacagggattcgaaaccatgcttttgtgaactgtatatgctatctcttttacatgtttccataccggaaaaaaaagttaggaaaaagaacatgtttccataacggcaaaaaaataggtcccaccgggattcgaacccaggtcgccagattcaaagtctggagtgctaaccactacactatggaacccgtttatgtcataatgtggacaattaattattaaaccccagcatatggaaccaaatccatttcccactacctcgaaagtcgaaaccatttggtctagtcagaacaccgaagtatccaacgaatccattgtaattttgttgcacccgaaatggcgccgctacggaataagcggcctttatgcttggcgcgagagttcttccttcaaaatcgatcttcaacgttcaaattcaaaatgctacccccgtccattgtaaattgtatgtgttcagtattttatatataatgtttatgtttacagaaaaattaaaacgatatataatctggaatggagagagtagcaacatccaatccaatccatcgacccgaccgagagaaacgggagggcacgctgctatcagctacctgctccatagtgtcgcaaactcacagtcacagcagtcatcacatgcctcaaggctggaagggcagaacacttttttcttgacgcgtgtatctcagcgaccggcgccatcagtttgttttcgttccacgcccccacgcccaatgcgcccccaccgtccaggtccagccgtacacgtgcgtccggccggggatcgcagcagccgcacgcactggctgctgggctcggacggcgctgcccctgccgcagcgcagccatagattggaattgcggcgacgtggcctcctggcagcccaccctgaacaggcgcaccctatcctccgcctcgccgctcgccaatggacacctccactagtccacttggcgccactccagcttcacgctgcccgcgtctgtgattcccattcataagagctggccacgcagcctcggagcaacagacccccgcccacgaccacacgcagcaagcagcagccgcgcgctcgcctaggactaccgactacgagtagtgctcagctagcctccatcctcagcgcgcgagtggctgctgcgcattcctctctttatccgtctctgtgcttgtttaatttgtgttcgttcgtgagtaggcggccggcacatatggcagcgcagtccatgctcatgtcgaccagcgtcaacggcggccgcgcgctgccgtctcttcaggccgtccggccggctccatacccgcggctaccgctgccgtcgtcgtcgtcctcgtcagctggctacaggcactccaagtccgtctccgtgaagacgctcgccctgttcggcaaatccaaggtcaagaccgcgccggcgaagaaggtccgtgacccgtgcatggctagctagctagctagctgcacgtacactactgtactgctgctgtgccgtgtgttatgtgcacgcatgcatgctcctggctagttttattctattccctgtcatgtgttgctatgtgtgtgtatatatatgatggttttgctgtacaggctgctgcgcccaagcccaagccgaaggttgaggatggtatcttcggcacgtccggcgggatcggtttcaccaaggagaacgagctgttcgtcggccgcgttgccatgcttggctttgccgtgagtatagtcaaaagcactgcatctgcatatggagtgtgttcatgcacaccggaaggaattactccgtccctccgaaattataagtcgttgacgacacggccttcaaaatacaattttaaccatttaaaaattaaaataaactcttaatatttatgtttttttaaatactttcttaaataaattggtgtataacacgtgactattagtttttaaaactaaataataaaacggttatttatagtcaaagttttataagcttgatttaattcttatccaaaagatttataatttagacttggaggggtagaaggaagttgttcttatatatactctctgcgtttacatatatatgacgtttagaataagttagttagctttttatctaactagttttgttttaaacattatattacaaattaaaaatggagatagtgtaacagatgcaatatctttcgtattgaccaatataattagaggagtacacaccatcgatatctggtaatatattaggatatatatttatcttagcaaaaaaaaacatagccattcatttttgtgatagctgacgccgccgtagcatgatatggatatggacatacatatggtagtcgcagttacctgtgattttgtaaagttagcagcttcatgggttaaattcagcagggtaggaaaaaaagaggagtggtgggtggaagaaaattccaaacggtcaaaataattaacataaactactcacttaatttgaacctaaaataaattactcaattacacctctgctgctagcactctcccaaaataaataaattatagattgtctaatccacgcattattttgttatgcaaatctgctcgtgtatgtattattgtttcaaaccaaaaacggtagctagcattgccagttcaagctagcgaggatttattgacactgcttggcttcaggcatcgctacttggagaggccattaccgggaagggcatccttgcgcagctgaacctggagacgggcatccccatctacgaggcggagcccctcctcctgttcttcatcctcttcaccctccttggcgccatcggcgctctcggagaccgcggccgcttcgtcgacgaggaggtcaccggcctcgacaaggccgtcatccaacccggcaagggcttccgcggcgccctcggcctcagcgagggaggtaattaaaaaaagaaaaagaagctcacgacacctccgcatgcagctgatgagctgtggttgattgcttgatttgagtattttttatataataatgatggtgcctgcggtgcatgaacaatgcagggccgctgttcgggttcaccaagtcgaacgagctgttcgtggggcggatggcgcagctgggcgtggccttctccatcatcggcgagatcatcacggggaagggcgcgctggcgcagctcaacatcgagacgggggtgcccatcaacgagatagagccgctcgtcatcttcaacgtcctcttcttcttcatcgccgccatcaaccccggcaatggcaggttcatcatcggcgaggaagaggaataggctgctggctgctgctgctggcctcggacatgagtcgtgtgcgtttacttttacctgtgactgtgcgtctcggtgcgcgcgcgtaagtgctttgctgtgtatcaattactcgtgatgagacctcgaaggtagggtgtcgcgttgggttgtctgcttgtagaaacaagagcttatatataaattactactagctaatatgccatggttgcaacga

SEQ ID NO: 99 - DNA, 20 bp, synthetic construct
  source 1..20: mol_type = other DNA
taatacgactcactataggg

SEQ ID NO: 100 - DNA, 20 bp, synthetic construct
  source 1..20: mol_type = other DNA
gtttaagagctatgctggaa

SEQ ID NO: 101 - DNA, 90 bp, synthetic construct
  source 1..90: mol_type = other DNA
aaaagcaccgactcggtgccactttttcaagttgataacggactagccttatttaaacttgctatgctgtttccagcatagctcttaaac

SEQ ID NO: 102 - DNA, 60 bp, synthetic construct
  source 1..60: mol_type = other DNA
taatacgactcactataggggcgagacactaaaatacattgtttaagagctatgctggaa

SEQ ID NO: 103 - DNA, 22 bp, synthetic construct
  source 1..22: mol_type = other DNA
ctacctatgcaacatgtgaccc